(19) United States
(12) Patent Application Publication
(10) Pub. No.: US 2019/0316085 A1
(43) Pub. Date: Oct. 17, 2019

(54) Methods Of T-Lymphocyte Expansion

(71) Applicant: Cambridge Enterprise Limited, Cambridge (GB)

(72) Inventors: Randall Scott JOHNSON, Cambridge (GB); Dr. Asis PALAZON, Cambridge (GB); Petros Andreas TYRAKIS, Cambridge (GB)

(73) Assignee: Cambridge Enterprise Limited, Cambridge (GB)

(21) Appl. No.: 15/772,782

(22) PCT Filed: Oct. 13, 2016
(86) PCT No.: PCT/EP2016/074625

§ 371 Date: May 1, 2018

(30) Foreign Application Priority Data
Nov. 2, 2015 (GB) 1519340.2

(51) Int. Cl. C12N 5/0783; A61K 35/17

(52) U.S. Cl. CPC C12N 5/0636; C12N 2501/999; C12N 2500/32; A61K 35/17

(57) ABSTRACT

This invention relates to methods for the expansion of T-lymphocytes with a memory-like phenotype in which the intracellular concentration of a memory induction compound, such as 2-hydroxyglutarate (2HG), is increased in order to facilitate the maintenance of a memory-like phenotype. This may be useful for example, in cellular immunotherapy. The memory induction compound may the formula (I) wherein: p is 0 or 1, and when p is 0, Y is —CH₂— or —C═, and when p is 1, Y is selected from —CH—, CH₂, —NH—, —S, and -0-; —R¹ is —H, —(CH₂)ₙCH₃, —(CH₂)ₙCH₂CO₂H, —CH₂Ph or —CH₂PhOCH₂Ph; and when Y is —CH—, CH₂, —NH—, —S, or —O—, X is a single bonded group selected from —H, —OH, —NH₂, —SH, —(CH₂)ₙCH₃ —(CH₂)ₙCH₂CO₂H, —F, —Cl, —Br, and —I, or a double bonded group selected from ═O and ═S; and when Y is a double bonded —C═, X is —H; and each n is independently 0 to 12.

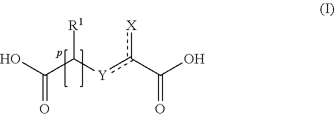

### FIELD

This invention relates to methods for the in vitro expansion of T-lymphocytes, for example, for use in adoptive T cell therapy.

### BACKGROUND

Cellular immunotherapy-based strategies using adoptive transfer of naturally occurring or T-cell receptor (TCR) engineered autologous T-lymphocytes are a promising new way of delivering effective clinical responses in the context of cancer and other conditions.

Currently, T-cells are isolated from peripheral blood or tumours of a patient, manipulated and expanded in vitro, followed by re-infusion.

There are two main approaches to performing adoptive T cell therapy; isolation of tumour infiltrating lymphocytes (TILs) from the patient, in vitro expansion and infusion; or isolation of T lymphocytes, modification of their T-cell receptors (TCR) in vitro to recognize a particular tumour antigen and infusion. The chimeric antigen receptor (CAR) approach is generally effective when the target antigen is robustly expressed on malignant cells (i.e. CD19 antigen expressed in B cell leukaemia). The TIL approach is most effective in types of cancer which display high mutation rates (such as melanoma) because a full, personalized repertoire of TCRs is represented in the re-infused T cells.

Both approaches rely on the generation of effector T lymphocytes, which are short lived, highly cytolytic T cells with limited renewal and proliferative capacity. The responses obtained with adoptive T cell therapy are impressive in the short term, but long term success has so far been limited. Preclinical studies have shown that a robust memory response is essential in order for a response to be durable, but to date there are no reports of the generation of autologous memory cells.

The main challenge of adoptive T cell therapy is therefore the generation of durable immune responses. One reason for the difficulty in generating such responses is the failure of the transferred cells to persist after transfer. This lack of persistence arises because activated T-cells become terminally differentiated effector cells with a limited renewal and proliferative capacity during the expansion phase in vitro. Methods of expanding T-lymphocytes in vitro whilst retaining a memory-like phenotype and without terminal differentiation would therefore be useful in increasing the persistence of the transferred cells in adoptive T cell therapy and generating durable immune responses.

### SUMMARY

This invention relates to the finding that increasing the intracellular concentration of memory induction compounds, such as 2-hydroxyglutarate (2HG), in T-lymphocytes facilitates the maintenance of a memory-like phenotype.

Increasing the intracellular concentration of a memory induction compound in T-lymphocytes may therefore be useful in the expansion of cell populations, for example for use in the generation of durable T-cell responses in cellular immunotherapy.

An aspect of the invention provides a method of expanding a population of T-lymphocytes comprising;
providing an initial population of T-lymphocytes,
increasing the intracellular concentration of a memory induction compound in the T-lymphocytes, and
culturing the T-lymphocytes,
thereby producing an expanded population of T-lymphocytes.

The number and/or proportion of memory-like T-lymphocytes in the expanded population may be increased relative to the initial population.

Another aspect of the invention provides a method of treatment comprising;
providing an initial population of T-lymphocytes obtained from a donor individual,
increasing the intracellular concentration of a memory induction compound in the T-lymphocytes,
culturing the T-lymphocytes to produce an expanded population, and;
administering the expanded population of T-lymphocytes to a recipient individual.

Another aspect of the invention provides a culture medium for the expansion of T-lymphocytes, said culture medium comprising a memory induction compound or a pro-form of a memory induction compound.

Another aspect of the invention provides the use of a memory induction compound or a pro-form thereof to maintain a memory-like phenotype in T-lymphocytes cultured in vitro.

The memory induction compound of the invention may be an organic diacid, or a mono- or diester form of such a compound. Preferably, the memory induction compound has the formula (I):

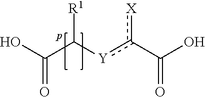

wherein:
p is 0 or 1, and when p is 0, Y is —CH₂— or —C═, and when p is 1, Y is selected from —CH—, CH₂, —NH—, —S, and —O—;
R¹ is selected from —H, —(CH₂)ₙCH₃, —(CH₂)ₙCH₂CO₂H, —CH₂Ph or —CH₂PhOCH₂Ph; and when Y is —CH—, CH₂, —NH—, —S, and —O—, X is a single bonded group selected from —H, —OH, —NH₂, —SH, —(CH₂)ₙCH₃—(CH₂)ₙCH₂CO₂H, —F, —Cl, —Br, and —I, or X is a double bonded group selected from ═O and ═S;
and when Y is a double bonded —C═, X is —H;
each n is independently 0 to 12,
and the mono- and diester forms thereof.

Preferred memory induction compounds include 2-hydroxyglutarate (2HG), succinate and fumarate.

2-hydroxyglutarate (2HG) may include R-2-hydroxyglutarate (R-2HG, also known as D-2-hydroxyglutarate), S-2-hydroxyglutarate (S-2HG, also known as L-2-hydroxyglutarate) or mixtures thereof.

### BRIEF DESCRIPTION OF FIGURES

FIG. 1 shows that VHL-HIF signalling regulates 2-hydroxyglutarate levels. Principal component analysis of Vhl^{Fl/Fl}, Vhl^{-/-} and Hif1α^{-/-}Vhl^{-/-} CD8⁺ T-lymphocyte metabolomes. Percentage variance of each PC is shown in parenthesis.

FIG. 2 shows that VHL-HIF signalling regulates 2-hydroxyglutarate levels. FIG. 2A shows metabolites ranked in order of decreasing p-value from metabolomic screen. FIG. 2B shows the relative level of 2HG in Vhl^{Fl/Fl}, Vhl^{-/-} and Hif1α^{-/-}Vhl^{-/-} CD8+ T-lymphocytes. Each dot represents an individual mouse. FIG. 2C shows LC-MS/MS quantification of total 2HG (S-2HG+R-2HG) levels in Vhl^{Fl/Fl} and Vhl^{-/-} CD8⁺ T-lymphocytes activated with αCD3 and αCD28 antibodies for 48 h and then cultured in IL-2 for a further 5 days; n=4 individual mice per genotype. FIGS. 2D and 2E show LC-MS/MS quantification of total 2HG (S-2HG+R-2HG) levels in (D) RCC4 and (E) 786-0 cells with or without reconstitution of VHL; n=3; EV=empty vector. FIG. 2F shows LC-MS/MS quantification of total 2HG (S-2HG+R-2HG) levels in MEFs with or without deletion of VHL; n=2-4 individual isolations. p-values are shown for every panel where applicable (Two-tailed Student's t-test for pairwise comparisons and one-way ANOVA for multiple comparisons. Error bars denote s.d. and each dot in B and C represents an individual mouse. ns=non-significant.

FIG. 3 shows that hypoxic induction of 2-hydroxyglutarate depends on HIF-1α, not HIF-2α, in CD8⁺ T-lymphocytes. FIG. 3A shows LC-MS/MS quantification of total 2HG (S-2HG+R-2HG)in CD8⁺ T-lymphocytes isolated from C57BL/6J mice and activated with αCD3+αCD28 antibodies for 48 h. Cells were then cultured with IL-2 in either 21% or 1% oxygen for a further 48 h; n≥11 mice per condition. FIG. 3B shows the total intracellular concentration of total 2HG (S-2HG+R-2HG), determined from by normalization to cell volume. FIG. 3C shows ¹H-NMR analysis for total 2HG (S-2HG+R-2HG) from CD8⁺ T-lymphocytes cultured as in 3A. FIG. 3D shows enantioselective analysis for S-2HG and R-2HG in intracellular metabolite extracts from CD8⁺ T-lymphocytes cultured as in FIG. 3a; n=23 mice. FIG. 3E shows the intracellular concentration of total 2HG (S-2HG+R-2HG) in CD8⁺ T-lymphocytes isolated from Hif1α^{fl/fl} and Hif1α^{-/-} mice, activated with αCD3+αCD28 antibodies for 48 h and cultured for a further 48 h with IL-2 in either 21% or 1% oxygen; n=4 mice per genotype. FIG. 3F shows intracellular concentration of total 2HG (S-2HG+R-2HG) in CD8⁺ T-lymphocytes isolated from Hif2α^{fl/fl} and Hif2α^{-/-} mice, activated with αCD3+αCD28 antibodies for 48 h and cultured for a further 48 h with IL-2 in either 21% or 1% oxygen; n=4 mice per genotype. FIG. 3G shows intracellular amount of total 2HG (S-2HG+R-2HG) in naïve and activated CD8⁺ T-lymphocytes, isolated from C57BL/6J mice, at indicated times following activation. n≥4 mice per time point. p-values are shown for every panel where applicable (Two-tailed Student's t-test for pairwise comparisons (A, B), one-way ANOVA for multiple comparisons (D) and two-way ANOVA for grouped data (E, F). Error bars denote s.d.; each dot in A, B, D and G represents an individual mouse.

FIG. 4 shows that glutamine is the source of 2HG in hypoxic CD8+ T-lymphocytes. FIGS. 4A and 4B show the ¹³C-isotopologue profile of total 2HG (S-2HG+R-2HG) in CD8⁺ T-lymphocytes activated and cultured as in FIG. 3A in (A) 1% and (B) 21% oxygen with unlabelled substrates, U-¹³C-glucose or U-¹³C-glutamine; n=7 mice per condition. Error bars denote s.d.

FIG. 5 shows that the HIF-1α-PDK1 axis controls the production of total 2HG (S-2HG+R-2HG) in hypoxic CD8⁺ T-lymphocytes. FIG. 5A shows LC-MS/MS quantification of total intracellular glutamate, succinate, fumarate and malate levels at day 4 after activation, in αCD3+αCD28 activated CD8⁺ T-lymphocytes isolated from C57BL/6J mice, in both 21% and 1% oxygen as in FIG. 3A; n=7 mice. FIG. 5B shows LC-MS/MS quantification of total intracellular glutamate levels in CD8⁺ T-lymphocytes isolated from Hif1α^{fl/fl} and Hif1α^{-/-} mice, activated with αCD3+αCD28 antibodies for 48 h and cultured for a further 48 h with IL-2 in either 21% or 1% oxygen; n=4 mice per genotype. FIG. 5C shows LC-MS/MS quantification of total intracellular glutamate levels in CD8⁺ T-lymphocytes isolated from Hif2α^{fl/fl} and Hif2α^{-/-} mice, activated with αCD3+αCD28 antibodies for 48 h and cultured for a further 48 h with IL-2 in either 21% or 1% oxygen; n=4 mice per genotype. FIG. 5D shows an immunoblot of cytosolic fractions for phospho-PDH E1α (S232) and total PDH-E1a in CD8⁺ T-lymphocytes, activated with αCD3+αCD28 antibodies for 48 h and cultured for a further 48 h with IL-2 in 1% oxygen and the indicated concentration of dichloroacetate (DCA). FIGS. 5E and 5F show (E) intracellular concentration of total 2HG (S-2HG+R-2HG) or (F) total intracellular glutamate levels in CD8⁺ T-lymphocytes, activated with αCD3+αCD28 antibodies for 48 h and cultured fora further 48 h with IL-2 in either 21% or 1% oxygen with 5 mM DCA; n=4 mice per group. p-values are shown for every panel where applicable (Two-tailed Student's t-test for pairwise comparisons (3A,) and two-way ANOVA for grouped data (3B to 3F)). Error bars denote s.d. and each dot in A represents an individual mouse. ns=non-significant.

FIG. 6 shows immunoblot analysis of nuclear and cytosolic fractions, prepared from CD8⁺ T-lymphocytes cultured in both 21% and 1% oxygen.

FIG. 6A showns immunoblot analysis for HIF-1α, HDAC1, phospho-PDH E1α (S232) and total PDH-E1α in response to increasing concentrations of S-2HG-octyl ester or R-2HG-octyl ester, or 10 mM of the free acid forms of S-2HG or R-2HG for 16 hours. Cells were activated for 48 h with αCD3+αCD28 antibodies and then expanded for a further 4 days in the presence of IL-2 followed by treatment with the indicated concentration of S-2HG-octyl ester or R-2HG octyl ester, or 10 mM of the free acid forms of S-2HG or R-2HG for 16 hours. The arrow indicates HIF-1α protein.

FIG. 6B shows immunoblot analysis on nuclear extracts for HDCA1, Histone H3, HIF-1α and HIF-2a proteins in response to 0.5 mM S-2HG octyl ester or 0.5 mM R-2HG-octyl ester after 1 day or 7 days of treatment. The arrow indicates HIF-2a protein.

FIG. 7 shows that S-2HG-octyl ester and/or R-2HG-octyl ester drive metabolic alterations in CD8+ T-lymphocytes. FIG. 7A shows glucose consumption, 7B lactate production and 7C VEGF production in CD8⁺ T-lymphocytes treated with 0.5 mM S-2HG-octyl ester or 0.5 mM R-2HG-octyl ester for 16 hours as in FIG. 6a. Each dot represent one donor mouse n≥16 mice. P-values are shown for each panel; one-way ANOVA for multiple comparisons (A, B and C). n.s.=non significant.

FIG. 8 shows that S-2HG-octyl ester and R-2HG-octyl ester drive the acquisition of memory associated properties in CD8+ T-lymphocytes. FIG. 8A shows specific killing of EG7-OVA cells by OT-I CD8⁺ T-lymphocytes. Total splenocytes were activated for 48 h with 1000 nM SIINFEKL and then expanded for a further 4 days in the presence of IL-2 followed by treatment 0.5 mM of S-2HG-octyl R-2HG-octyl ester ester for 24 h. OT-I CD8⁺ T-lymphocytes were incubated with target and control cells for 4 hours. n=3 mice per condition. FIG. 8B shows the amount of IFN-γ and FIG. 8C IL-2 protein in the media of wild type CD8⁺ T-lymphocytes treated for 24 h with 0.5 mM S-2HG-octyl ester or 0.5 mM R-2HG-octyl ester or vehicle. Cells were activated for 48 h with αCD3+αCD28 antibodies and then expanded for a further 4 days in the presence of IL-2 followed by treatment with the indicated concentration of S-2HG-octyl ester or R-2HG-octyl ester for 24 hours. n≥16 mice. FIG. 8D shows the survival of OT-1CD8⁺ T-lymphocytes activated with 1000 nM SIINFEKL peptide and cultured for 7 days with or without 0.5 mM S-2HG-octyl ester or R-2HG-octyl ester in the absence of IL-2 supplementation from day 0. n=4 mice per condition.

FIGS. 8E, 8F and 8G show expression of II2(8E), Ifng (8F), and Eomes (8G) mRNA in CD8⁺ T-lymphocytes activated for 48 h with αCD3+αCD28 antibodies and then expanded for a further 2 days in the presence of IL-2. Cells were then treated for either 24 h or 7 days with or without 0.5 mM S-2HG-octyl ester or R-2HG-octyl ester. n≥4 mice per group. FIG. 8H shows CD44 and CD62L expression on the surface of OT-1CD8⁺ T-lymphocytes, activated with varying SIINFEKL doses and treated from day 0 for either 4 or 7 days with or without 0.5 mM S-2HG-octyl ester or R-2HG-octyl ester in the presence of IL-2. n=4 mice per group. *p<0.05, **p<0.01, ***p<0.001, p<0.0001. FIG. 8I shows that S-2HG-octyl etser and R-2HG-octyl ester induce the expression of other memory associated genes, including Ccr7.

FIG. 9 shows that S-2HG-octyl ester and R-2HG-octyl ester decrease proliferation after activation in CD8⁺ T-lymphocytes. One way ANOVA for mutiple comparisons(B). Error bars denote s.d. and each dot in B represents an individual mouse.

FIG. 9A shows CFSE dilution assay at day 3 of CD8⁺ T-Iymphocytes activated with αCD3+αCD28 antibodies and cultured with 0.5 mM S-2HG-octylester, 0.5 mM R-2HG-octyl ester or vehicle in the presence of IL-2 from day 0. Data are representative of 4 mice.

FIG. 9B shows mean fluorescence intensity of CFSE dilution assay in 9A at day 3. n=4 mice per condition.

FIG. 9C shows fold-change in viable cell number of CD8⁺ T-lymphocytes activated with αCD3+αCD28 antibodies for 48 h and then cultured with IL-2 for a further 48 h. Cells were then treated with 0.5 mM S-2HG-octyl ester, 0.5 mM R-2HG-octyl ester or vehicle and counting was performed at indicated time points after initiation of treatment. n=8 mice per condition.

FIG. 10 shows that S-2HG-octyl ester and R-2HG-octyl ester promote the formation of CD44High and CD62L^{High} OT-I CD8⁺ T-lymphocytes.

FIG. 10A shows an illustration outlining the workflow for the experiment in FIG. 10B.

FIG. 10B shows CD44 and CD62L expression on the surface of OT-I CD8⁺ T-lymphocytes activated with 1000 nM SIINFEKL and treated for either 4, 6 and 8 days with 0.5 mM S-2HG-octyl ester, 0.5 mM R-2HG-octyl ester or vehcile, in the presence of IL-2. Data are representative of n=4 individual mice.

FIG. 11 shows that HIF-1α is needed for the in vitro down-regulation of CD62L in activated CD8⁺ T-lymphocytes.

FIG. 11A shows illustration outlining the workflow for the experiments in FIGS. 11B and C.

FIG. 11B shows CD44 and CD62L surface expression on Hi1α^{fl/fl} and Hif1α^{-/-} CD8⁺ T-lymphocytes treated with 0.5 mM S-2HG-octyl ester, 0.5 mM R-2HG-octyl ester or vehicle at 1, 7 and 10 days following treatment. Data are representative of 3 mice per genotype.

FIG. 11C shows CD44 and CD62L surface expression on Hif2α^{fl/fl} and Hif2α^{-/-} CD8⁺ T-lymphocytes treated with 0.5 mM S-2HG-octyl ester, R-2HG-octyl ester or vehicle at 1, 7 and 10 days following treatment. Data are representative of 2 mice per genotype.

FIG. 12 shows that S-2HG-octyl ester and R-2HG-octyl ester induce memory-like surface markers in a dose-dependent manner and that this is reversible. p-values are shown for every panel where applicable, one-way ANOVA for multiple comparisons (D, E) Error bars denote s.d. ns=non-significant.

FIG. 12A shows CD62L surface expression on OT-I CD8⁺ T-lymphocytes as a function of S-2HG-octyl ester and R-2HG-octyl ester concentration after 4 days of treatment. The dotted line represent the level of CD62L on vehicle treated cells on day 4. Cells were activated with 1000 nM SIINFEKL peptide and cultured in the presence of IL-2; n=3 mice.

FIG. 12B shows CD62L surface expression on OT-I CD8⁺ T-lymphocytes as a function of S-2HG-octyl ester and R-2HG-octyl ester concentration after 7 days of treatment. The dotted line represent the level of CD62L on vehicle treated cells on day 7. Cells were activated with 1000 nM SIINFEKL peptide and cultured in the presence of IL-2; n=3 mice. FIG. 12C shows illustration outlining the experimental workflow for data presented in FIGS. 12D and 12E.

FIG. 12D shows % CD62L^{High} CD8⁺ T-lymphocytes, treated for 7 days with either vehicle or 0.5 mM R-2HG-octyl ester, followed by either washout of R-2HG-octyl ester from the R-2HG-octyl ester treated cells, or addition of 0.5 mM R-2HG-octyl ester to the vehicle treated cells and follow up every 3rd day, for 9 more days. n=4 mice.

FIG. 12E shows % CD62L^{High} CD8⁺ T-lymphocytes, treated for 7 days with either vehicle or 0.5 mM S-2HG-octyl ester, followed by either washout of S-2HG-octyl ester from the S-2HG-octyl ester treated cells, or addition of 0.5 mM S-2HG-octyl ester to the vehicle treated cells and follow up every 3^{rd} day, for 9 more days. n=4 mice.

FIG. 13 shows that the treatment of CD8⁺ T-cells in vitro with either S-2HG-octyl ester or R-2HG octyl ester induces the formation of memory cells. FIG. 13A shows an outline of the experimental work flow for the memory recall experiment in FIGS. 13B, 13C and 13D. FIGS. 13B and 13C show representative flow cytometry plots of (B) CD8⁺CD45.1⁺ (C) or Kb/SIINFEKL Pentamer⁺CD45.1⁺ T-lymphocytes, in the spleens of vaccinated litter mate CD45.2 mice, 37 days after adoptive transfer of SIINFEKL-activated OT-I CD8⁺CD45.1⁺ T-lymphocytes, expanded with 0.5 mM S-2HG-octyl ester, 0.5 mM R-2HG-octyl ester or vehicle in the presence of IL-2 for 7 days. FIGS. 13D-F show (13D) the number of recovered CD45.1⁺CD8⁺ T-lymphocytes per spleen and (13E) % CD45.1⁺ cells of the total CD8⁺ population in each spleen or (13F) number of recovered Kb/SIINFEKL Pentamer⁺CD45.1⁺ T-lymphocytes per spleen 7 days after vaccination (37 days after the CD8+ T-cells were transferred into the mice). n=6 mice per group and error bars denote the s.e.m.

FIG. 14 shows that treatment with S-2HG-octyl ester, and R-2HG-octyl ester, induces the expression of pluripotency associated genes needed for stemness. Activated CD8⁺ T-lymphocytes were treated with 0.5 mM of either S-2HG-octyl ester or R-2HG-octyl ester for 1 day or 7 days in the presence of IL2. The induction seen with R-2HG-octyl ester is weaker than with S-2HG-octyl ester. Each dot represents an individual donor mouse, n=4 mice. error bars denote the s.d.

FIG. 15 shows that treatment with 100 μM to 500 μM S-2HG-octyl ester, and R-2HG-octyl ester, does not inhibit mTOR signalling to form memory.

FIG. 16 shows that treatment of CD8+ T-cells in vitro with α-ketoglutarate octyl ester does not induce the formation of memory cells.

FIG. 17 shows that the treatment of CD8+ T-cell in vitro with monomethylfumarate or dimethylsuccinate induces the formation of memory cells, whereas treatment with α-ketoglutarate octyl ester does not. FIG. 17A shows an outline of the experimental work flow for FIGS. 17B and 17C. FIG. 17B shows representative flow cytometry plots of CD8⁺CD45.1⁺ T-lymphocytes, in the spleens of vaccinated litter mate CD45.2 mice, 37 days after adoptive transfer of SIINFEKL-activated OT-I CD8⁺CD45.1⁺ T-lymphocytes, expanded with 0.5 mM monomethylfumarate, 0.5 mM dimethylsuccinate, 0.5 mM R-2HG-octyl ester, 0.5 mM S-2HG-octyl ester, 0.5 mM α-ketoglutarate octyl ester or vehicle in the presence of IL-2 for 7 days.

FIG. 17 shows that monomethylfumarate or dimethylsuccinate also induce the formation of CD44^{High} and CD62L^{High} OT-I CD8⁺ T-lymphocytes in vitro.

FIG. 17A shows an illustration outlining the workflow for the experiment in FIGS. 17B and 17C.

FIG. 17B shows CD44 and CD62L expression on the surface of OT-I CD8⁺ T-lymphocytes activated with 1000 nM SIINFEKL and treated for either 7 days with S-2HG-octyl ester, R-2HG-octyl ester, αKG-octyl ester, monomethylfumarate, dimethylsuccinate or vehicle, in the presence of IL-2. Data are representative of n=3 individual mice.

FIG. 17C shows the mean fluorescence intensity of CD62L and associated statistics. Error bars represent s.d. and each dot represents an individual mouse. One way ANOVA.

FIG. 18 shows the flow cytometric characterisation of indicated phenotypic markers on Hif1α^{fl/fl} (n=4) and Hif1α^{fl/fl} dlck^{cre} (n=4) CD8⁺ T-lymphocytes treated for 7 days with 500 μM S-2HG-octyl ester. Gated on live CD8⁺ cells. Each dot represents an individual mouse.

FIG. 19A shows the validation of L2hgdh-FLAG expression in CD8⁺ T-lymphocytes from C57BL/6J mice by immunoblot analysis for FLAG. The arrow indicates L2hgdh-FLAG protein.

FIG. 19B shows CD62L expression on CD8⁺ T-lymphocytes, isolated from C57BL/6J mice and cultured in 21% (n=7) or 1% (n=3) oxygen for 7 days after transduction with retrovirus containing empty or L2hgdh-FLAG overepression vectors. Each pair of dots represents an individual mouse. Gated on live, CD8⁺GFP⁺ cells.

FIG. 19C shows representative flow cytommetry plots of KLRG1 vs CD127 expression on CD8⁺ T-lymphocytes, isolated from C57BL/6J mice (n=4) and cultured in 21% oxygen with or without 300 μM S-2HG-octyl ester for 7 days after transduction with retrovirus containing empty or L2hgdh-FLAG overepression vectors. Associated statistics are shown and each pair of dots represents an individual mouse. Gated on live, CD8⁺GFP⁺ cells.

FIG. 20A shows qPCR validation of L2hgdh knockdown in CD8⁺ T-lymphocytes isolated from C57BL/6J mice.

FIG. 20B shows the intracellular amount of S-2HG in CD8⁺ T-lymphocytes in response to shRNA against L2hgdh in both 21% and 1% oxygen conditions (n=4).

FIG. 20C shows CD62L surface expression in response to shRNA against L2hgdh (n=4). Representative flow cytometry histogram of CD62L surface levels on transduced (GFP⁺) CD8⁺ T-lymphocytes in response to shScramble or shL2hgdh in 21% or 1% oxygen is shown on the right. Each dot represents an individual mouse.

FIG. 20D shows CD127 surface expression in response to shRNA against L2hgdh (n=4). Representative flow cytometry histogram of CD127 surface levels on transduced (GFP⁺) CD8⁺ T-lymphocytes in response to shScramble or shL2hgdh in 21% or 1% oxygen is shown on the right. Each dot represents an individual mouse.

FIG. 21A shows a diagram outlining the homeostatic proliferation experiments. CD45.1.1 or CD45.1.2 OT-I CD8⁺ T-lymphocytes were activated with 1000 nM SIINFEKL peptide and cultured with or without 300 μM S-2HG-octyl ester for 9 days. Cells from each group were mixed 1:1 and labelled with CFSE prior to transfer into sub-lethally irradiated CD45.2.2 mice. 7 days later, mice were sacrificed and the presence of CD451.1 and CD45.1.2 CD8⁺ T-lymphocytes was enumerated in spleen by flow cytometry. Representative flow cytometry plots are shown for each pool before and after adoptive transfer. Flow cytometry plots show viable CD8⁺ cells.

FIG. 21B shows the recovery of adoptively co-transferred CD45.1⁺OT-I CD8⁺ T-lymphocytes, pre-treated with or without 300 μM S-2HG-octyl ester for 9 days, from spleens of sub-lethally irradiated CD45.2.2 mice (n=6), 7 days after adoptive transfer.

FIG. 21C shows the in vivo CFSE levels in cells from FIG. 21B, on day 7 after adoptive transfer.

FIG. 21D shows the % of transferred cells from FIG. 21B that have divided 0-9 times in vivo.

FIG. 22A shows representative flow cytometry plots and associated statistics of recovered adoptively transferred CD45.1⁺OT-I CD8⁺ T-lymphocytes, pre-treated with or without 300 μM S-2HG-octyl ester for 9 days, from spleens of CD45.2.2 mice (n=6) 30 days after transfer. Flow cytomotery plots are gated on live cells.

FIG. 22B shows representative phenotypic analysis of recovered cells from FIG. 22A, at day 30 after transfer, relative to naïve cells (n=6).

FIG. 23A shows a diagram outlining the recall experiments. CD45.1.1 or CD45.1.2 OT-I CD8⁺ T-lymphocytes were activated with 1000 nM SIINFEKL peptide and cultured with or without 300 μM S-2HG-octyl ester for 9 days. Cells from each group were mixed 1:1 prior to transfer into CD45.2.2 mice. 30 days later, these recipient mice were vaccinated with SIINFEKL-loaded dendritic cells to induce recall and the presence of CD451.1 and CD45.1.2 CD8⁺ T-lymphocytes was enumerated in spleen, lymphnodes and liver by flow cytometry 7 days later.

FIG. 23B shows representative flow cytometry plots of recalling CD45.1⁺CD8⁺ T-lymphocytes in indicated organs on day 7 post vaccination (day 37 post transfer).

FIG. 23C shows the recovery of adoptively co-transferred CD45.1⁺OT-I CD8⁺ T-lymphocytes, pre-treated with or without 300 μM S-2HG-octyl ester for 7 days, from spleens, lymphnodes and livers of vaccinated CD45.2.2 mice (n=6), 37 days after transfer.

FIG. 24A shows C57BL/6J mice bearing subcutaneous EG7-OVA tumours for 12 days followed by intravenous injection of no T-cells (n=7) or 0.7×10⁶ OT-I CD8⁺ T-lymphocytes previously cultured with (n=6) or without (n=6) S-2HG-octyl ester. Mice were lymphodepleted with sub-lethal irradiation before receivening intravenous injection of OT-I CD8⁺ T-lymphocytes. Error bars denote s.e.m. *p<0.05, ns=non-significant.

FIG. 24B shows lymphoreplete C57BL/6J mice bearing subcutaneous EG7-OVA tumours for 9 days followed by intravenous injection of no T-cells (n=6) or 1.0×10⁶ OT-I CD8⁺ T-lymphocytes previously cultured with (n=6) or without (n=6) S-2HG-octyl ester. Error bars denote s.e.m. *p<0.05, ns=non-significant.

FIG. 25A shows representative flow cytometry plots for the surface markers CCR7 and CD45RO on purified human CD8⁺ activated and expanded in vitro in the absence (vehicle control) or presence of 600 μM S-2HG-octyl ester for 14 days.

FIG. 25B shows representative flow cytometry plots for the surface markers CCR7 and CD45RO on purified human CD8⁺ activated and expanded in vitro in the absence (vehicle control) or presence of 800 μM R-2HG-octyl ester for 14 days. Numbers in dot plots represent the percentage of cells present in the corresponding quadrant defined by CCR7 and CD45RO expression.

### DETAILED DESCRIPTION

This invention relates to the in vitro expansion of T-lymphocyte populations, for example for use in cellular immunotherapy. Increasing the intracellular concentration of a memory induction compound, such as 2HG, succinate or fumarate, during expansion is shown herein to facilitate the formation of memory-like T-lymphocytes and inhibit terminal differentiation into non-proliferative effector T-lymphocytes (e.g. cytotoxic T lymphocytes; CTLs).

Preferably, the increased intracellular concentration of the memory induction compound in the T-lymphocytes does not inhibit mTOR or mTOR signalling pathways in the T-lymphocytes. For example, the phosphorylation of p70S6 kinase and 4E-BP1 in the T-lymphocytes may be unaffected by the increased intracellular concentration of the memory induction compound. Methods of the invention therefore allow the expansion of memory-like T-lymphocytes in vitro without inhibition of mTOR signalling.

In addition to expression of the markers CD62L^{high}, CCR7^{high}, CD44^{high}, memory-like T-lymphocytes expanded as described herein may also display increased long-term proliferation and viability relative to effector T-lymphocytes expanded in the absence of the memory induction compound.

Preferably, the increased intracellular concentration of the memory induction compound in the T-lymphocytes during culture and expansion prevents differentiation into effector cells and the loss of memory-like properties.

In all of the aspects of the invention described herein, the T-lymphocytes may be CD4+ T-lymphocytes or more preferably CD8⁺ T-lymphocytes.

CD8⁺ and CD4⁺ T-lymphocytes are part of the adaptive immune system. The normal function of CD8⁺ T-lymphocytes is to kill cancer cells and cells infected with intracellular pathogens, such as bacteria and viruses. CD8⁺ T-lymphocytes express the heterodimeric receptor CD8. CD8⁺ T-lymphocytes recognise peptides presented by MHC Class I molecules on the surface of antigen presenting cells. During this recognition, the CD8 heterodimer binds to a conserved portion (the α3 region) of MHC Class I. CD4⁺ T-lymphocytes are frequently characterised as T helper cells and facilitate the production of antibodies by B cells, enhance and maintain the responses of CD8⁺ T-lymphocytes, and regulate macrophage activity. CD4⁺ T-lymphocytes express the receptor CD4. CD4⁺ T-lymphocytes assist the interaction of the T cell receptor with antigen presenting cells and bind to MHC Class II molecules.

The initial population may comprise T-lymphocytes specific for a target antigen i.e. they may be capable of recognising and being activated by a specific peptide antigen displayed by an antigen presenting cell (APC) in the context of a class I MHC molecule.

In some embodiments, the initial population may be polyclonal. The cells in the population may recognise different epitopes of the same antigen when displayed in the context of class I MHC molecules or may recognise epitopes of different antigens when displayed in the context of class I MHC molecules.

In other embodiments, the initial population may be monoclonal i.e. the cells in the population may recognise the same epitope of the same target antigen when displayed in the context of class I MHC molecules.

The T-lymphocytes in the initial population may be a mixture of undifferentiated, partially differentiated and fully differentiated cells. For example, the initial population may comprise naïve, memory- and effector T cells.

The initial population of T-lymphocytes may be obtained from a donor individual.

Any suitable donor individual may be used. In some embodiments, the T-lymphocytes may be obtained from a donor individual suffering from a disease condition, such as viral, bacterial or fungal infection or cancer, or from a healthy individual, for example a healthy individual who is human leukocyte antigen (HLA) matched (either before or after donation) with an individual suffering from such a condition.

The initial population of T-lymphocytes may be isolated or otherwise obtained from appropriate samples from the donor individual e.g. samples from lymphoid tissue such as spleen or lymph nodes or from blood or tumour samples. Suitable isolation techniques are well known in the art and include, for example fluorescent activated cell sorting (FACS: see for example, Rheinherz et al (1979) PNAS 76 4061), cell panning (see for example, Lum et al (1982) Cell Immunol 72 122) and isolation using antibody coated magnetic beads (see, for example, Gaudernack et al 1986 J Immunol Methods 90 179). Conveniently, CD8⁺ T-lymphocytes may be isolated using anti-CD8 antibodies and CD4⁺ T-lymphocytes may be isolated using anti-CD4 antibodies. For example, the sample may be incubated with magnetic beads coated with anti-CD8 or anti-CD4 antibodies and the beads isolated using magnetic separation.

In some embodiments, the initial population of T-lymphocytes may be comprised in a sample of cells from the donor individual. The sample of cells may be a heterogeneous sample comprising other cell types, such as B cells, dendritic cells and macrophages, in addition to the initial population of T-lymphocytes.

In some preferred embodiments, a method described herein may comprise activating T-lymphocytes in the initial population.

The T-lymphocytes may be activated in a separate culture step before, preferably immediately before the intracellular concentration of the memory induction compound in the T-lymphocytes levels is increased. Alternatively, the T-lymphocytes may be activated at the same time as the intracellular concentration of the memory induction compound in the T-lymphocytes is increased i.e. in the same culture step.

T-lymphocytes may be activated by any convenient technique. In some preferred embodiments, the T-lymphocytes may be activated by exposure to a T cell receptor (TCR) agonist. A method as described herein may further comprise;
exposing the T-lymphocytes to a TCR agonist.

Suitable TCR agonists include TCR ligands, such as a peptide displayed on a class I or II MHC molecule on the surface of a presentation cell.

A presentation cell may include any nucleated cell. The peptide/MHC class I or class II complex may be naturally expressed by the presentation cell or may be heterologous to the presentation cell and expressed by means of a heterologous encoding nucleic acid previously introduced into the cell by recombinant means.

In some embodiments, the presentation cell may be an antigen presenting cell (APC). Suitable APCs that express MHC class II include natural APCs, such as macrophages, monocytes, B cells and dendritic cells (DC) or artificial APCs, for example fibroblasts or other cells which have been engineered to express MHC class I or II and optionally ICAM-70.

Suitable presentation cells may be isolated from a sample obtained from a donor individual.

In some embodiments, sample from the donor individual which comprises the initial population of T-lymphocytes may further comprise presentation cells. A peptide antigen introduced to the cells in the sample is displayed in an MHC class I or II complex on the surface of the presentation cells in the sample. The presentation cells displaying the MHC class I or II complex then activate the T-lymphocytes in the sample.

A method described herein may further comprise;
exposing a presentation cell, for example an antigen presenting cell (APC), such as a dendritic cell, to an exogenous peptide antigen in vitro, such that the antigen is displayed by MHC class I or II molecules on the surface of the presentation cell, and
culturing the presentation cell in vitro with the T-lymphocytes, such that the T-lymphocytes are activated by the antigen displayed by the MHC class I or II molecules.

In other embodiments, the T-lymphocytes may be cultured in the absence of a presentation cell in a culture medium which comprises the activating peptide antigen, which can then be taken up and displayed in combination with an MHC class I molecule on the surface of the T-lymphocytes themselves.

Suitable TCR agonists also include soluble factors, such as agonistic specific binding members, which are present in the culture medium and which stimulate the TCR, either on their own or when cross-linked to the presentation cell via an immunoglobulin Fc receptor, such as CD32, which is displayed on the surface of the presentation cell.

Suitable agonistic specific binding members include anti-TCR antibodies.

An anti-TCR antibody may specifically bind to a component of the TCR, such as εCD3, αCD3 or αCD28. Anti-TCR antibodies suitable for TCR stimulation are well-known in the art (e.g. OKT3) and available from commercial suppliers (e.g. eBioscience CO USA).

In some preferred embodiments, the T-lymphocytes may be activated by exposure to anti-αCD3 antibodies and anti-αCD28 antibodies.

Activation, expansion and increasing the concentration of the memory induction compound may be performed sequentially in separate culture media or simultaneously, in the same culture medium.

The T lymphocytes with an increased intracellular concentration of memory induction compound may be cultured using any convenient technique to produce the expanded population.

The T lymphocytes may be cultured as described herein in any suitable system, including stirred tank fermenters, airlift fermenters, roller bottles, culture bags or dishes, and other bioreactors, in particular hollow fibre bioreactors. The use of such systems is well-known in the art.

Numerous culture media suitable for use in the proliferation of T lymphocytes ex vivo are available, in particular complete media, such as AIM-V, Iscoves medium and RPMI-1640 (Invitrogen-GIBCO). The medium may be supplemented with other factors such as serum, serum proteins and selective agents. For example, in some embodiments, RPMI-1640 medium containing 2 mM glutamine, 10% FBS, 25 mM HEPES, pH 7.2, 1% penicillin-streptomycin, and 55 μM β-mercaptoethanol and optionally supplemented with 20 ng/ml recombinant IL-2 may be employed. The culture medium may be supplemented with the agonistic or antagonist factors described above at standard concentrations which may readily be determined by the skilled person by routine experimentation.

Conveniently, cells are cultured at 37° C. in a humidified atmosphere containing 5% CO₂ in a suitable culture medium.

Methods and techniques for the culture of T lymphocytes and other mammalian cells are well-known in the art (see, for example, Basic Cell Culture Protocols, C. Helgason, Humana Press Inc. U.S. (15 Oct. 2004) ISBN: 1588295451; Human Cell Culture Protocols (Methods in Molecular Medicine S.) Humana Press Inc., U.S. (9 Dec. 2004) ISBN: 1588292223; Culture of Animal Cells: A Manual of Basic Technique, R. Freshney, John Wiley & Sons Inc (2 Aug. 2005) ISBN: 0471453293, Ho W Y et al J Immunol Methods. (2006) 310:40-52)

The initial population of T-lymphocytes obtained from the donor individual is cultured in vitro such that the cells proliferate to expand the initial population. During the in vitro culture, the intracellular concentration of the memory induction compound in the T-lymphocytes is increased.

The intracellular concentration of the memory induction compound may be increased in the T-lymphocytes by any suitable technique.

A memory induction compound may be a diacid, or a mono- or diester form of the compound. Acid here refers to a carboxylic acid group, —COOH, and the carboxylate form also. Thus, salt forms of the compounds are also contemplated. A diacid therefore contains two carboxylic acid groups, which are each optionally in acid, salt or ester form.

The compound may include additional functionality, such as hydroxyl, amino, thiol, or halo functionality. The compound may include alkenyl (or alkenylene) functionality. The compound may include additional carboxylic acid groups. However, the number of carboxylic acid groups is usually 2.

In some embodiments, one or both acid groups is an α,β-unsaturated acid.

In some embodiments, one or both acid groups is a saturated acid.

The carboxylic acid groups may be connected via an alkylene, heteroalkylene or alkenylene linker, which linker may be optionally substituted, such as optionally substituted with one or more of hydroxyl, amino (—NH₂), thiol, halo, phenyl and substituted phenyl.

An alkylene linker may be linear or branched, such as linear, and may be C₁₋₂₀ alkylene, such as C₂₋₂₀, such as C₂₋₁₀, such as C₂₋₆, such as C₂₋₄, such as C₂₋₃.

A heteroalkylene linker is an alkylene linker where one or more, such as one, carbon atom in an alkylene linker is replaced with a heteroatom group O, S, or NH. The heteroalkylene linker may be C₃₋₂₀, such as C_{3_10}, such as C₃₋₆, such as C₃₋₄, such as C₃. The heteroatom is not bonded to a carboxylic acid group.

An alkenylene linker may be linear or branched, such as linear, and may be C2-20 alkylene such as C₂₋₂₀, such as C₂₋₁₀, such as C₂₋₆, such as C₂₋₄, such as C₂₋₃.

Where two carboxylic groups are connected by an alkylene linker, the compound may be referred to as a saturated dicarboxylic acid.

Where two carboxylic groups are connected by an alkenylene linker, the compound may be referred to as an unsaturated dicarboxylic acid.

The compound may be a saturated dicarboxylic acid, such as a linear dicarboxylic acid, or the salt or ester forms thereof. The saturated dicarboxylic acid may be unsubstituted or monosubstituted.

The compound may be an unsaturated dicarboxylic acid, such as a monounsaturated dicarboxylic acid, or the salt or ester forms thereof. An unsaturated dicarboxylic acid may be a linear unsaturated dicarboxylic acid, or the salt or ester forms thereof.

The compound may contain further carboxylic acid functionality, although it is typical for the compound to have only two carboxylic acid groups. The compound contains a carbonyl group within each carboxylic acid, and therefore a diacid has two carbonyl groups. Preferably, the compound does not contain other carbonyl functionality.

The compound preferably does not include keto functionality, and more preferably does not contain keto ester functionality, such as alpha- -keto ester functionality. The inventors have found that the keto ester compound alpha ketoglutarate does not provide good activity

Typically the ester of a carboxylic acid is an alkyl ester, such as a C₁₋₁₀ alkyl ester, such as a C₁₋₈ alkyl ester. The worked examples in the present case include methyl and octyl esters. The alkyl group may be linear or branched, such as linear.

In some embodiments, the compound may have a molecular weight of at most 200, at most 150 or at most 100. The compound may have from 4 to 30 carbon atoms, such as from 4 to 20 carbon atoms. The compound may 4 or 5 oxygen atoms.

The diacid and ester forms of the compounds are commercially available, or may be prepared using standard synthesis methods.

In some embodiments, the compound is 2-hydroxyglutarate, fumarate and/or succinate, and the salt and ester forms thereof.

In some embodiments, the compound is not α-ketoglutarate, such as the compound is not α-ketoglutarate octyl ester.

Preferably, the memory induction compound has the formula (I):

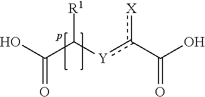

wherein:
p is 0 or 1, and when p is 0, Y is —CH₂— or —C═, and when p is 1, Y is selected from —CH—, CH₂, —NH—, —S, and —O—;
—R¹ is —H, —(CH₂)ₙCH₃, —(CH₂)ₙCH₂CO₂H, —CH₂Ph or —CH₂PhOCH₂Ph;
and when Y is —CH—, CH₂, —NH—, —S, or —O—, X is a single bonded group selected from —H, —OH, —NH₂, —SH, —(CH₂)ₙCH₃—(CH₂)ₙCH₂CO₂H, —F, —Cl, —Br, and —I, or a double bonded group selected from ═O and ═S;
and when Y is a double bonded —C═, X is —H; and
each n is independently 0 to 12,

and the mono- and diester forms thereof, such as the alkyl mono- and diester forms thereof.

In some embodiments, the memory induction compound as described herein may have the formula (II):

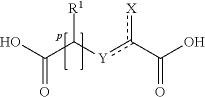

wherein:
p is 1;
Y is selected from —CH—, CH₂, —NH—, —S, and —O—;
—R¹ is —H;
Y is selected from —CH—, CH₂, —NH—, —S, and —O—;
X is a single bonded group selected from —H, —OH, —NH₂, —SH, —(CH₂)ₙCH₃—(CH₂)ₙCH₂CO₂H, —F, —Cl, —Br, and —I;
each n is independently 0 to 12,

and the mono- and diester forms thereof, such as the alkyl mono- and diester forms thereof.

In some embodiments, the memory induction compound as described herein may have the formula (III):

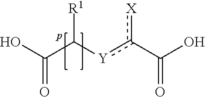

wherein:
p is 1;
—R¹ is —H, —(CH₂)ₙCH₃, —(CH₂)ₙCH₂CO₂H, —CH₂Ph or —CH₂PhOCH₂Ph;
Y is selected from —CH—,CH₂, —NH—, —S, and —O—;
X is a double bonded group selected from ═O and ═S; and
each n is independently 0 to 12,

and the mono- and diester forms thereof, such as the alkyl mono- and diester forms thereof.

In some embodiments, the memory induction compound as described herein may have the formula (IV):

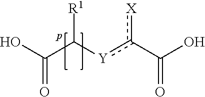

wherein:
p is 0;
X is H; and
Y is selected from —CH—, CH₂, —NH—, —S, and —O—,

and the mono- and diester forms thereof, such as the alkyl mono- and diester forms thereof.

Preferred memory induction compounds include 2-hydroxyglutarate (2HG), succinate and fumarate.

In some embodiments, the memory induction compound is not α-ketoglutarate.

A memory induction compound may include free acids and pharmaceutically acceptable salts thereof.

In some preferred embodiments, the memory induction compound is 2HG. 2HG may include S-2-hydroxyglutarate (S-2HG), R-2-hydroxyglutarate (R-2HG) or mixtures thereof. A mixture may contain a defined ratio of the enantiomers. For example, a mixture may comprise 30% S-2HG and 70% R-2HG. In some especially preferred embodiments, 2HG is R-2HG.

In some embodiments, the T-lymphocytes are cultured in a hypoxic environment to increase the intracellular concentration of 2HG. A hypoxic environment may include any environment with less than 21% oxygen, less than 15% oxygen, or less than 10% oxygen, for example, 10%, 5% or 1% oxygen. Hypoxic environments increase the intracellular production of 2HG.

In other embodiments, the T-lymphocytes are cultured in a medium that increases the intracellular concentration of the memory induction compound.

A suitable culture medium may comprise the memory induction compound or, more preferably a pro-molecule thereof.

During culture in the medium, the memory induction compound or pro-molecule thereof crosses the cell membrane and enters the T-lymphocytes, thereby increasing the intracellular concentration of the memory induction compound.

The culture medium may comprise 10 μM to 10 mM of the memory induction compound or pro-form thereof, preferably about 0.1-0.5 mM.

In some embodiments, the passage of the memory induction compound or pro-form across the cell membrane into the T-lymphocytes may be increased by electroporation. For example, the memory induction compound or pro-form from the culture medium may be introduced into the T-lymphocytes by electroporation, thereby increasing the intracellular concentration of the memory induction compound. Suitable electroporation techniques are well-known in the art.

In some embodiments, the passage of the memory induction compound or pro-form thereof across the cell membrane into the T-lymphocytes may be increased by treating the cells with a solvent which increases cell permeability. Suitable solvents are well-known in the art and include DMSO, oils and alcohols. For example, the permeability of the T-lymphocytes to the memory induction compound or pro-form thereof may be increased using a solvent, thereby increasing the intracellular concentration of the memory induction compound.

In some embodiments, the passage of the memory induction compound or pro-form thereof across the cell membrane into the T-lymphocytes may be increased by modifying the cells to express a molecular transporter, such as a 2HG transporter. Suitable transporters are well-known in the art and include carboxylate transporters. For example, the intracellular concentration of the memory induction compound may be increased by culturing cells modified to express a molecular transporter in the presence of the memory induction compound.

In some embodiments, the culture medium may comprise a pro-form of a memory induction compound.

A pro-form of a memory induction compound is a precursor molecule that is converted into the memory induction compound within the T-lymphocytes (e.g. by an intracellular enzyme). Preferably, the cell permeability of the pro-form of the memory induction compound is higher than the cell permeability of the memory induction compound i.e. it has an increased ability to cross the plasma membrane of the T-lymphocytes.

Examples of pro-forms of memory induction compounds may include pro-2HG, pro-fumarate and pro-succinate. Pro-2HG may include pro-S-2HG, pro-R-2HG and mixtures of pro-R-2HG and pro-S-2HG.

The pro-form of a memory induction compound may include free acids and pharmaceutically acceptable salts thereof.

The pro-form may comprise the memory induction compound conjugated to one or more cell permeable moieties. For example, pro-S-2HG may comprise S-2HG conjugated to one or more cell permeable moieties and pro-R-2HG may comprise R-2HG conjugated to one or more cell permeable moieties.

A cell-permeable moiety is a molecule or chemical group which facilitates or increases the penetration of molecules through cell membranes.

A range of cell permeable moieties suitable for conjugation to the memory induction compound are known in the art and may be employed in accordance with the invention.

Suitable cell permeable moieties include hydrophobic moieties such as lipids, fatty acids, steroids and bulky aromatic or aliphatic compounds including alkyl groups, preferably but not limited to, C₁ to C₂₄, preferably C₁ to C₁₂ alkyl groups, including mono- or di-methyl, ethyl, propyl, butyl, pentyl, hexyl, heptyl, octyl, nonyl, docyl, undecyl and dodecyl groups; moieties which may have cell-membrane receptors or carriers, such as steroids, vitamins and sugars, natural and non-natural amino acids, such as deoxyglucosamine, oligonucleotides, such as oligoguanidinium, and peptides, such as transporter peptides and cell-penetrating peptides (CPPs).

Cell permeable moieties may include Lipofectamine™, Transfectace™, Transfectam™, Cytofectin™, DMRIE, DLRIE, GAP-DLRIE, DOTAP, DOPE, DMEAP, DODMP, DOPC, DDAB, DOSPA, EDLPC, EDMPC, DPH, TMADPH, CTAB, lysyl-PE, DC-Cho, -alanyl cholesterol; DOGS, DPPES, DOPE, DMAP, DMPE, DOGS, DOHME, DPEPC, Pluronic™, Tween™, BRIJ, plasmalogen, phosphatidylethanolamine, phosphatidylcholine, glycerol-3-ethylphosphatidylcholine, dimethyl ammonium propane, trimethyl ammonium propane, diethylammonium propane, triethylammonium propane, dimethyldioctadecylammonium bromide, a sphingolipid, sphingomyelin, a lysolipid, a glycolipid, a sulfatide, a glycosphingolipid, cholesterol, cholesterol ester, cholesterol salt, oil, N-succinyldioleoylphosphatidylethanolamine, 1,2-dioleoyl-sn-glycerol, 1,3-dipalmitoyl-2-succinylglycerol, 1,2-dipalmitoyl-sn-3-succinylglycerol, 1-hexadecyl-2-palmitoylglycerophosphatidylethanolamine, palmitoylhomocystiene, N,N′-Bis (dodecyaminocarbonylmethylene)-N,N′-bis((—N,N,N-trimethylammoniumethyl-ami nocarbonylmethylene)ethylenediamine tetraiodide; N₅N″-Bis(hexadecylaminocarbonylmethylene)-N, N′, N″-tris((—N,N,N-trimethylammonium-ethylaminocarbonylmethylenediethylenetri amine hexaiodide; N,N Bis(dodecylaminocarbonylmethylene)-N,N^{M}-bis((—N,N,N-trimethylammonium ethylaminocarbonylmethylene)cyclohexylene-1,4-diamine tetraiodide; 1,7,7-tetra-((—N,N,N,N-tetrametihiylammoniumethylamino-carbonylmethylene)-3-hexadecylaminocarbonyl-methylene-1,3,7-triaazaheptane heptaiodide; N₅N₅N′,N′-tetra((—N, N, N-trimethylammonium-ethylaminocarbonylmethylene)-N′-(1₅2-dioleoylglycero-3-phosphoethanolamino carbonylmethylene)diethylenetriam ine tetraiodide; dioleoylphosphatidylethanolamine; fatty acid, lysolipid, phosphatidylcholine, phosphatidylethanolamine, phosphatidylserine, phosphatidylglycerol, phosphatidylinositol, sphingolipid, glycolipid, glucolipid, sulfatide, glycosphingolipid, phosphatidic acid, palmitic acid, stearic acid, arachidonic acid, oleic acid, cholesterol, tocopherol hemisuccinate, a lipid with an ether-linked fatty acid, a lipid with an ester-linked fatty acid, a polymerized lipid, diacetyl phosphate, stearylamine, cardiolipin, a phospholipid with a fatty acid of 6-8 carbons in length, a phospholipid with asymmetric acyl chains, 6-(5-cholesten-3b-yloxy)-I-thio-b-D-galactopyranoside, digalactosyldiglyceride, 6-(5-cholesten-3b-yloxy)hexyl-6-amino-6-deoxy-1-thio-b-D-galactopyranoside, 6-(5-cholesten-3b-yloxy)hexyl-6-amino-6-deoxyl-1-thio-a-D-mannopyranoside, 12-(((7′-diethylamino-coumarin-3-yl)carbonyl)methylamino)-octadecanoic acid; N-[12-(((7′-diethylaminocoumarin-3-yl)carbonyl)methyl-amino) octadecanoyl]-2-aminopalmitic acid; cholesteryl)4′-trimethyl-ammonio)butanoate; N-succinyldioleoyl-phosphatidylethanolamine; 1,2-dioleoyl-sn-glycerol; I\hat{ }-dipalmitoyl-sn-Succinyl-glycerol; I,3-dipalmitoyl-2-succinylglycerol, I-hexadecyl-2-pahnitoylglycero-phosphoethanolamine, and palmitoylhomocysteine.

CPPs are hydrophobic or basic peptides which cross the plasma membrane in a receptor- and energy-independent manner. Suitable CPPs include membrane-translocating sequence (MTS), trans-activating transcriptional activator (TAT: YGRKKRRQRRR), Penetratin (RQIKIYFQNRRMKWKK), CAR (CARSKNKDC), oligoarginine (e.g. R₈) Xentry™ (LCLRPVG).) transportan, transportan 10, MPG and Pep-1.

The cell permeable moiety may be linked to the memory induction compound in the pro-form by a labile bond that is subject to intracellular cleavage within the T-lymphocytes to release memory induction compound.

Suitable labile bonds include ester bonds, ether bonds, amide bonds, ketone bonds and disulphide bonds.

Preferably, the labile bond is an ester bond. Ester bonds may be cleaved by intracellular esterases in the T-lymphocytes to release a memory induction compound, such as 2HG, from a pro-form, such as pro-2HG, inside the cells.

In some preferred embodiments, the pro-form is an alkyl ester of the memory induction compound, preferably but not limited to, mono- or di-methyl, ethyl, propyl, butyl, pentyl, hexyl, heptyl, octyl, nonyl, docyl, undecyl and dodecyl esters of the memory induction compound, most preferably C₈ alkyl ester of the memory induction compound (2HG octyl ester).

Suitable pro-forms of memory induction compounds include dimethylsuccinate, monomethylfumarate, R-2HG octyl ester, S-2HG octyl ester and mixtures thereof.

A T-lymphocyte with increased intracellular levels of memory induction compound may display one or more of increased phosphorylation of PDH-E1α, increased glucose uptake, increased lactate secretion, increased VEGF production, reduced lytic ability, decreased secretion of interferon-γ (IFN-γ), increased production of interleukin-2 (IL-2) and increased survival in culture in the absence of IL-2 supplementation.

Increasing intracellular levels of the memory induction compound during expansion of a population of T-lymphocytes, causes the cells to adopt a memory-like phenotype rather than an effector phenotype.

A memory-like phenotype may comprise expression of one, two, three or all four of the markers CD62^{high}, CD44^{high}, CCR7⁺ and CD45RO⁺. For example, a memory-like phenotype may comprise expression of the markers CD62L^{high}, CCR7^{high} and CD44^{high}; CD62L^{high} and CD44^{high}; and/or CCR7⁺ and CD45RO⁺.

A memory-like phenotype may further comprise an increased ability to survive in a host for long period of time and/or greater recall upon vaccination relative to an effector phenotype.

Memory induction compounds, such as 2HG, are shown herein to increase the number of memory-like cells in the expanded population. For example, the number of T-lymphocytes in the expanded population that are memory-like T-lymphocytes may be increased relative to;
(i) control populations cultured in the absence of memory induction compound, and/or
(ii) the initial population

Memory induction compounds, such as 2HG, are shown herein to increase the proportion of memory-like cells in the expanded population. For example, the proportion of T-lymphocytes in the expanded population that are memory-like T-lymphocytes may be increased relative to;
(i) control populations cultured in the absence of memory induction compound, and/or
(ii) the initial population

For example, after 7 days of culturing T-lymphocytes with an increased intracellular concentration of memory induction compound, at least 60%, at least 70%, at least 80% or at least 90% of the cells in the population may display a memory like phenotype. By comparison, after 7 days of culturing control T-lymphocytes without increased intracellular concentration of the memory induction compound, less than 30%, less than 20%, or less than 10% of the cells in the population may display a memory like phenotype.

A population of memory-like T-lymphocytes produced as described herein may be useful for adoptive cell transfer in a range of applications, including cancer immunotherapy and vaccine development.

As described above, for some applications, the T-lymphocytes in the initial population may be polyclonal.

In some preferred embodiments, an initial population of polyclonal T-lymphocytes may be tumour infiltrating lymphocytes (TILs).

A suitable population of TILs may be isolated from a tumor sample from an individual with a cancer condition. The population of TILs isolated from the sample may comprise a repertoire of TCRs that is specific to the antigens expressed by the tumor in the individual.

Expansion of the population of TILs as described herein may produce an expanded population of memory-like T-lymphocytes which express the tumor specific repertoire of TCRs. The expanded population may be administered to the donor individual (i.e. autologous T cell transfer) to treat the cancer condition in the individual.

Any cancer condition described herein may be treated using TILs. Preferred cancers for treatment include cancers with high mutation rates, e.g. melanoma, lung, cervical cancer and digestive tract cancers, such as colorectal cancer.

As described above, for some applications, the T-lymphocytes in the initial population may be monoclonal (i.e. antigen-specific).

Following the isolation of the initial population, the T-lymphocytes may be modified, for example, to be specific for or recognise a target antigen, for example a tumor antigen.

The T-lymphocytes may be engineered or modified before, during or after the concentration of memory induction compound is increased in the cells, preferably before.

For example, the T-lymphocytes may be modified to express a heterologous antigen receptor such as a chimeric antigen receptor, T body receptor or heterologous αβTCR heterodimer. The heterologous receptor may be specific for an antigen, for example a tumor antigen.

Heterologous receptors suitable for expression in T-lymphocytes may have a known specificity and avidity for a selected target antigen.

In some embodiments, cancer cells may express one or more antigens that are not expressed by normal somatic cells in an individual (i.e. tumour antigens). Tumour antigens may elicit immune responses in the individual. In particular, tumour antigens may elicit T cell-mediated immune responses against cancer cells in the individual that express the one or more tumour antigens. One or more tumour antigens may be selected as a target antigen for heterologous receptors on modified T-lymphocytes. T-lymphocytes modified to express the heterologous receptors may be expanded as described herein and administered to the individual for treatment of the cancer condition.

Tumour antigens expressed by cancer cells may include, for example, cancer-testis (CT) antigens encoded by cancer-germ line genes, such as MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, GAGE-I, GAGE-2, GAGE-3, GAGE-4, GAGE-5, GAGE-6, GAGE-7, GAGE-8, BAGE-I, RAGE-1, LB33/MUM-1, PRAME, NAG, MAGE-Xp2 (MAGE-B2), MAGE-Xp3 (MAGE-B3), MAGE-Xp4 (MAGE-B4), MAGE-C1/CT7, MAGE-C2, NY-ESO-I, LAGE-I, SSX-I, SSX-2(HOM-MEL-40), SSX-3, SSX-4, SSX-5, SCP-I and XAGE and immunogenic fragments thereof (Simpson et al. Nature Rev (2005) 5, 615-625, Gure et al., Clin Cancer Res (2005) 11, 8055-8062; Velazquez et al., Cancer Immun (2007) 7, 1 1; Andrade et al., Cancer Immun (2008) 8, 2; Tinguely et al., Cancer Science (2008); Napoletano et al., Am J of Obstet Gyn (2008) 198, 99 e91-97).

Other tumour antigens that may be expressed include, for example, overexpressed or mutated proteins and differentiation antigens particularly melanocyte differentiation antigens such as p53, ras, CEA, MUC1, PMSA, PSA, tyrosinase, Melan-A, MART-1, gp100, gp75, alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A11, hsp70-2, KIAAO205, Mart2, Mum-2, and 3, neo-PAP, myosin class I, OS-9, pml-RAR.alpha. fusion protein, PTPRK, K-ras, N-ras, Triosephosphate isomeras, GnTV, Herv-K-mel, NA-88, SP17, and TRP2-Int2, (MART-I), E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, alpha.-fetoprotein, 13HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB\170K, NY-CO-1, RCAS1, SDCCAG16, TA-90 (Mac-2 binding protein\cyclophilin C-associated protein), TAAL6, TAG72, TLP, TPS and tyrosinase related proteins such as TRP-1, TRP-2.

Other tumour antigens that may be expressed include out-of-frame peptide-MHC complexes generated by the non-AUG translation initiation mechanisms employed by “stressed” cancer cells (Malarkannan et al. Immunity 1999 June; 10(6):681-90).

Other tumour antigens that may be expressed are well-known in the art (see for example WO00/20581; Cancer Vaccines and Immunotherapy (2000) Eds Stern, Beverley and Carroll, Cambridge University Press, Cambridge) The sequences of these tumour antigens are readily available from public databases but are also found in a, WO 1994/005304 A1, WO 1994/023031 A1, WO 1995/020974 A1, WO 1995/023874 A1 and WO 1996/026214 A1.

T-lymphocytes may be genetically modified to express a heterologous antigen receptor using any convenient technique. For example, a heterologous nucleic acid, such as a nucleic acid construct or vector encoding the heterologous receptor may be introduced into the cells in the culture medium. This may be useful in altering the function or antigenic specificity of the T-lymphocytes, for example, by causing the non-effector T-cells to express a heterologous antigen receptor. For example, a construct encoding a heterologous antigen receptor such as a TCR or TCR subunit which is specific for a particular antigen, for example a disease-associated antigen, or a construct encoding a dominant negative form of a receptor, such as TGFβ receptor II, may be introduced into the cells. The genetic modification of T-cells to express heterologous antigen receptors and the subsequent use of such genetically modified T-cells in adoptive T-cell therapy are well known in the art. The genes encoding TCR specific for a variety of disease-associated antigens, in particular tumor associated antigens such as MART-1, gp100, and NY-ESO-1, are well known in the art.

When introducing or incorporating a heterologous nucleic acid into a cell, certain considerations must be taken into account, well known to those skilled in the art. The nucleic acid to be inserted should be assembled within a construct or vector which contains effective regulatory elements which will drive transcription in the target cell. Suitable techniques for transporting the constructor vector into the cell are well known in the art and include calcium phosphate transfection, DEAE-Dextran, electroporation, liposome-mediated transfection and transduction using retrovirus or other virus, e.g. vaccinia or lentivirus. For example, solid-phase transduction may be performed without selection by culture on retronectin-coated, retroviral vector-preloaded tissue culture plates.

Many known techniques and protocols for manipulation and transformation of nucleic acid, for example in preparation of nucleic acid constructs, introduction of DNA into cells and gene expression are described in detail in Protocols in Molecular Biology, Second Edition, Ausubel et al. eds. John Wiley & Sons, 1992.

Optionally following expansion, T-lymphocytes may be isolated and/or purified from the expanded population using any convenient technique, including FACS and antibody coated magnetic particles, as described above. For example, T-lymphocytes specific for target antigen may be isolated from the expanded population.

Following expansion in in vitro culture as described herein and optional isolation, the T-lymphocytes may be formulated into a pharmaceutical composition with a therapeutically acceptable excipient.

Pharmaceutical compositions suitable for administration (e.g. by infusion), include aqueous and non-aqueous isotonic, pyrogen-free, sterile injection solutions which may contain anti-oxidants, buffers, preservatives, stabilisers, bacteriostats, and solutes which render the formulation isotonic with the blood of the intended recipient; and aqueous and non-aqueous sterile suspensions which may include suspending agents and thickening agents. Examples of suitable isotonic vehicles for use in such formulations include Sodium Chloride Injection, Ringer's Solution, or Lactated Ringer's Injection. Suitable vehicles can be found in standard pharmaceutical texts, for example, Remington's Pharmaceutical Sciences, 18th edition, Mack Publishing Company, Easton, Pa., 1990.

In some preferred embodiments, the T-lymphocytes may be formulated into a pharmaceutical composition suitable for intravenous infusion into an individual.

The term “pharmaceutically acceptable” as used herein pertains to compounds, materials, compositions, and/or dosage forms which are, within the scope of sound medical judgement, suitable for use in contact with the tissues of a subject (e.g., human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Following expansion in vitro as described herein, the T-lymphocytes may be administered to a recipient individual. In some embodiments, the donor individual and the recipient individual are the same (i.e. the T-lymphocytes are obtained from an individual who is subsequently treated with the T-lymphocytes). In other embodiments, the donor and the recipient individual are different (i.e. the T-lymphocytes are obtained from one individual and subsequently used to treat a different individual). The donor and recipient individuals may be HLA matched to avoid GVHD and other undesirable immune effects.

Aspects of the invention relate to the use of populations of T-lymphocytes expanded as described herein in therapy, for example adoptive T cell therapy.

A method of treatment of an individual may comprise;
administering a population of T-lymphocytes expanded as described above to an individual in need thereof.

The population of T-lymphocytes may be administered intravenously, for example by infusion into the individual.

The population of T-lymphocytes may be autologous i.e. the T-lymphocytes were originally obtained from the same individual to whom they are subsequently administered (i.e. the donor and recipient individual are the same). A suitable population of CD4⁺ or CD8⁺ T-lymphocytes for administration to a recipient individual may be produced by a method comprising providing an initial population of T-lymphocytes obtained from the individual, increasing the intracellular concentration of memory induction compound in the T-lymphocytes, and culturing the CD4⁺ or CD8⁺ T-lymphocytes.

The population of T-lymphocytes may be allogeneic i.e. the T-lymphocytes were originally obtained from a different individual to the individual to whom they are subsequently administered (i.e. the donor and recipient individual are different). A suitable population of T-lymphocytes for administration to a recipient individual may be produced by a method comprising providing an initial population of T-lymphocytes obtained from a donor individual, increasing the intracellular concentration of memory induction compound in the T-lymphocytes, and culturing the T-lymphocytes.

Following administration, the recipient individual may exhibit a memory T-lymphocyte mediated immune response.

An individual suitable for treatment with T-lymphocytes as described herein may have a condition that is ameliorated by a T-lymphocyte mediated immune response.

The T-lymphocytes may be specific for one or more antigens that are associated with the disease.

In some embodiments, the individual may have an infection, for example a viral, bacterial or fungal infection, cancer or an autoimmune condition.

Cancer may be characterised by the abnormal proliferation of malignant cancer cells and may include leukaemias, such as AML, CML, ALL and CLL, lymphomas, such as Hodgkin lymphoma, non-Hodgkin lymphoma and multiple myeloma, and solid cancers such as sarcomas, skin cancer, melanoma, bladder cancer, brain cancer, breast cancer, uterus cancer, ovary cancer, prostate cancer, lung cancer, colorectal cancer, cervical cancer, liver cancer, head and neck cancer, oesophageal cancer, pancreas cancer, renal cancer, adrenal cancer, stomach cancer, testicular cancer, cancer of the gall bladder and biliary tracts, thyroid cancer, thymus cancer, cancer of bone, and cerebral cancer.

Cancer cells within an individual may be immunologically distinct from normal somatic cells in the individual (i.e. the cancerous tumour may be immunogenic). For example, the cancer cells may be capable of eliciting a systemic immune response in the individual against one or more antigens expressed by the cancer cells. The antigens that elicit the immune response may be tumour antigens or may be shared by normal cells.

An individual suitable for treatment as described above may be a mammal, such as a rodent (e.g. a guinea pig, a hamster, a rat, a mouse), murine (e.g. a mouse), canine (e.g. a dog), feline (e.g. a cat), equine (e.g. a horse), a primate, simian (e.g. a monkey or ape), a monkey (e.g. marmoset, baboon), an ape (e.g. gorilla, chimpanzee, orang-utan, gibbon), or a human.

In some preferred embodiments, the individual is a human. In other preferred embodiments, non-human mammals, especially mammals that are conventionally used as models for demonstrating therapeutic efficacy in humans (e.g. murine, primate, porcine, canine, or rabbit animals) may be employed.

In some embodiments, the individual may have minimal residual disease (MRD) after an initial cancer treatment.

An individual with cancer may display at least one identifiable sign, symptom, or laboratory finding that is sufficient to make a diagnosis of cancer in accordance with clinical standards known in the art. Examples of such clinical standards can be found in textbooks of medicine such as Harrison's Principles of Internal Medicine, 15th Ed., Fauci A S et al., eds., McGraw-Hill, New York, 2001. In some instances, a diagnosis of a cancer in an individual may include identification of a particular cell type (e.g. a cancer cell) in a sample of a body fluid or tissue obtained from the individual.

Treatment may be any treatment and therapy, whether of a human or an animal (e.g. in veterinary applications), in which some desired therapeutic effect is achieved, for example, the inhibition or delay of the progress of the condition, and includes a reduction in the rate of progress, a halt in the rate of progress, amelioration of the condition, cure or remission (whether partial or total) of the condition, preventing, delaying, abating or arresting one or more symptoms and/or signs of the condition or prolonging survival of a subject or patient beyond that expected in the absence of treatment.

Treatment as a prophylactic measure (i.e. prophylaxis) is also included. For example, an individual susceptible to or at risk of the occurrence or re-occurrence of cancer may be treated as described herein. Such treatment may prevent or delay the occurrence or re-occurrence of cancer in the individual.

In particular, treatment may include inhibiting cancer growth, including complete cancer remission, and/or inhibiting cancer metastasis. Cancer growth generally refers to any one of a number of indices that indicate change within the cancer to a more developed form. Thus, indices for measuring an inhibition of cancer growth include a decrease in cancer cell survival, a decrease in tumor volume or morphology (for example, as determined using computed tomographic (CT), sonography, or other imaging method), a delayed tumor growth, a destruction of tumor vasculature, improved performance in delayed hypersensitivity skin test, an increase in the activity of cytolytic T-lymphocytes, and a decrease in levels of tumor-specific antigens. Reducing immune suppression in cancerous tumors in an individual may improve the capacity of the individual to resist cancer growth, in particular growth of a cancer already present the subject and/or decrease the propensity for cancer growth in the individual.

The memory-like T-lymphocytes or the pharmaceutical composition comprising the memory-like T-lymphocytes may be administered to a subject by any convenient route of administration, whether systemically/peripherally or at the site of desired action, including but not limited to; parenteral, for example, by infusion, including intravenous infusion, in particular intravenous bolus infusion. Suitable infusion techniques are known in the art and commonly used in therapy (see, e.g., Rosenberg et al., New Eng. J. of Med., 319:1676, 1988).

Typically, the number of cells administered is from about 10⁵ to about 10¹⁰ per Kg body weight, typically 10⁸-10¹⁰ cells per individual, typically over the course of 30 minutes, with treatment repeated as necessary, for example at intervals of days to weeks. It will be appreciated that appropriate dosages of the memory-like T-lymphocytes, and compositions comprising the memory-like T-lymphocytes, can vary from patient to patient. Determining the optimal dosage will generally involve the balancing of the level of therapeutic benefit against any risk or deleterious side effects of the treatments of the present invention. The selected dosage level will depend on a variety of factors including, but not limited to, the activity of the particular cells, the route of administration, the time of administration, the rate of loss or inactivation of the cells, the duration of the treatment, other drugs, compounds, and/or materials used in combination, and the age, sex, weight, condition, general health, and prior medical history of the patient. The amount of cells and the route of administration will ultimately be at the discretion of the physician, although generally the dosage will be to achieve local concentrations at the site of action which achieve the desired effect without causing substantial harmful or deleterious side-effects.

While it is possible for memory-like T-lymphocytes to be administered alone, it may be preferable in some circumstances to administer the cells in combination with the target antigen, APCs displaying the target antigen, and/or IL-2 to promote expansion in vivo of the population of memory-like T-lymphocytes.

In some embodiments, the population of memory-like T-lymphocytes may be administered in combination with one or more other therapies, such as cytokine e.g. IL-2 administration, cytotoxic chemotherapy and radiation.

The one or more other therapies may be administered by any convenient means, preferably at a site which is separate from the site of administration of the memory-like T-lymphocytes. In some embodiments, IL-2 may be administered intravenously.

Administration of memory-like T-lymphocytes can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician.

Another aspect of the invention provides an expanded population of memory-like T-lymphocytes produced by a method described herein. Populations of memory-like T-lymphocytes produced by the present methods are described elsewhere herein and are CD62L^{high} and lack effector functions, such as CTL activity and IFN-gamma expression.

Another aspect of the invention provides an expanded population of memory-like T-lymphocytes produced by a method described herein for use in a method of treatment as described herein.

Another aspect of the invention provides the use of an expanded population of memory-like T-lymphocytes produced by a method described herein in the manufacture of a medicament for use in a method of treatment as described herein.

Another aspect of the invention provides a culture medium for the expansion of T-lymphocytes comprising a memory induction compound or a pro-form thereof.

For example, the medium may comprise succinate, pro-succinate, fumarate, pro-fumarate, S-2HG, R-2HG, pro-R-2HG and/or pro-S-2HG.

A preferred medium may comprise pro-2HG, for example 2HG octyl ester.

The medium may comprise a basal medium such as RPMI-1640 supplemented with additional factors, such as glucose, amino acids such as glutamine, HEPES, pH 7.2, antibiotics, such as penicillin and streptomycin, and 3-mercaptoethanol.

In some embodiments, the medium may be a chemically defined medium. A CDM is a nutritive solution for culturing cells which contains only specified components, preferably components of known chemical structure. A CDM is devoid of components which are not fully defined, for example serum or proteins isolated therefrom, such as Foetal Bovine Serum (FBS), Bovine Serum Albumin (BSA), and feeder or other cells. In some embodiments, a CDM may be humanised and may be devoid of components from non-human animals. Proteins in the CDM may be recombinant human proteins Suitable CDMs are well known in the art and described in more detail below.

The medium may be supplemented with serum or a serum substitute.

Optionally the medium may be supplemented with recombinant IL-2 or other cytokines Basal media and media components may be obtained from commercial sources (e.g. Gibco, Roche, Sigma, Europabioproducts, Cellgenix, Life Sciences).

The culture medium may be formulated in deionized, distilled water. The culture medium will typically be sterilized prior to use to prevent contamination, e.g. by ultraviolet light, heating, irradiation or filtration. The culture medium may be frozen (e.g. at −20° C. or −80° C.) for storage or transport. The culture medium may contain one or more antibiotics to prevent contamination.

The culture medium may be a 1× formulation or a more concentrated formulation, e.g. a 2× to 250× concentrated medium formulation. In a 1× formulation each ingredient in the medium is at the concentration intended for cell culture, for example a concentration set out above. In a concentrated formulation one or more of the ingredients is present at a higher concentration than intended for cell culture. Concentrated culture media are well known in the art. Culture media can be concentrated using known methods e.g. salt precipitation or selective filtration. A concentrated medium may be diluted for use with water (preferably deionized and distilled) or any appropriate solution, e.g. an aqueous saline solution, an aqueous buffer or a culture medium.

The culture medium may be contained in hermetically-sealed vessels. Hermetically-sealed vessels may be preferred for transport or storage of the culture medium, to prevent contamination. The vessel may be any suitable vessel, such as a flask, a plate, a bottle, a jar, a vial or a bag.

Another aspect of the invention provides a kit for the in vitro expansion of T-lymphocytes comprising a memory induction compound or a pro-form thereof.

Another aspect of the invention provides the use of a memory induction compound or a pro-form thereof to maintain a memory-like phenotype in T-lymphocytes cultured in vitro.

For example, succinate, pro-succinate, fumarate, pro-fumarate, S-2HG, R-2HG, pro-R-2HG, pro-S-2HG or a mixture thereof may be used.

Other aspects of the invention relate to the finding that increases in the intracellular concentration of a memory induction compound in mammalian cells as described herein induce stem cell associated properties and/or pluripotency in the mammalian cells. This may be useful for example in reprogramming activated T-lymphocytes into T memory stem cells. T memory cells are antigen-experienced immune stem cells with self-renewal and multipotency capacity, which ensure life-long immunological memory by providing a continuous source of short-lived effector cells upon antigenic re-stimulation. Memory T cells share many features with pluripotent stem cells, including molecular signatures, transcriptional programs, asymmetric division and maintenance of telomere length (Fearon et al, Science 293, 248-250 (2001)). They also have the multipotent ability to give rise to a diverse progeny of effector and memory immune subsets from a single memory stem cell precursor (Graef et al (2014) Immunity 41. 116-126).

Among many factors, Oct3/4 and Nanog are critical in the maintenance of self-renewal and pluripotency of stem cells (Loh et al, Nature Genetics 38, 431-440 (2006)). Adult somatic cells can be reprogrammed into stem cells by the introduction of a specific set of genes. Initially, the expression of four genes was identified as minimal requirement for reprogramming Oct3/4, Sox2, Klf4 and c-myc (Takahashi, K. & Yamanaka, S. Cell 126, 663-676 (2006)). It is now well recognised that reprograming to pluripotency can be achieved by the expression of Oct3/4 alone (Kim et al, Nature 461, 649-653 (2009)).

An aspect of the invention provides the use of a memory induction compound or pro-form thereof as described above to induce stem cell associated properties and/or pluripotency in mammalian cells in in vitro cultures.

A method of inducing stem cell associated properties and/or pluripotency in mammalian cells or producing pluripotent stem cells may comprise;
providing an initial population of mammalian cells,
increasing the intracellular concentration of a memory induction compound in the mammalian cells, and
culturing the mammalian cells,
thereby producing an expanded population of mammalian cells with stem cell associated properties and/or pluripotency.

Memory induction compounds and methods of increasing intracellular concentrations of memory induction compounds are described in detail above.

Increasing the intracellular concentration of the memory induction compound in the somatic cells induces the expression of pluripotency reprogramming factors (Oct3/4, Sox2, Nanog and Klf4), leading to the reprogramming of the cells into pluripotent stem cells.

In some preferred embodiments, the number and/or proportion of pluripotent stem cells in the expanded population may be increased relative to the initial population.

Stem cell associated properties may include the expression of pluripotency reprogramming factors, such as Oct3/4, Sox2, Nanog and Klf4.

Suitable mammalian cells include somatic cells, such as T-lymphocytes.

In this context, an aspect of the invention provides a method for stem cell therapy comprising obtaining adult somatic cells from an individual and reprogramming the somatic cells into pluripotency by increasing the intracellular 2-HG concentration. The reprogrammed cells may be reinfused into the individual or differentiated into other cell types and reinfused into the individual.

Following reprogramming and optional differentiation, the cells may be administered to a recipient individual. In some embodiments, the donor individual and the recipient individual are the same (i.e. somatic cells for reprogramming are obtained from an individual who is subsequently treated with the pluripotent stem cells or cells differentiated therefrom). In other embodiments, the donor and the recipient individual are different (i.e. the somatic cells are obtained from one individual and the pluripotent stem cells are used to treat a different individual). The donor and recipient individuals may be HLA matched to avoid GVHD and other undesirable immune effects.

Populations of pluripotent stem cells produced as described herein may be used in therapy, for example stem cell therapy. A method of treatment of an individual may comprise;
administering a population of pluripotent stem cells produced as described above to an individual in need thereof.

In some embodiments, following reprogramming, the pluripotent stem cells may be differentiated in vitro to produce differentiated somatic cells. The differentiated somatic cells may then be used in therapy.

Other aspects and embodiments of the invention provide the aspects and embodiments described above with the term “comprising” replaced by the term “consisting of” and the aspects and embodiments described above with the term “comprising” replaced by the term “consisting essentially of”.

It is to be understood that the application discloses all combinations of any of the above aspects and embodiments described above with each other, unless the context demands otherwise. Similarly, the application discloses all combinations of the preferred and/or optional features either singly or together with any of the other aspects, unless the context demands otherwise.

Modifications of the above embodiments, further embodiments and modifications thereof will be apparent to the skilled person on reading this disclosure, and as such, these are within the scope of the present invention.

All documents and sequence database entries mentioned in this specification are incorporated herein by reference in their entirety for all purposes.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Certain aspects and embodiments of the invention will now be illustrated by way of example and with reference to the figures described above.

### Experiments

### 1. Materials and Methods

### 1.1 Isolation and Activation of CD8⁺ T-Lymphocytes

CD8⁺ T-lymphocytes were isolated from mouse spleens by positive selection. Incubation with MicroBeads conjugated to monoclonal anti-mouse CD8a (Ly-2; isotype: rat IgG2a) antibody (Miltenyi, 130-049-401) was followed by magnetic bead isolation on a MACS column. Unless otherwise stated, CD8⁺ T-lymphocytes were activated with plate-bound αCD3 (5 μg/ml) and soluble αCD28 (1 μg/ml) for 48 h. For activation of OT-I CD8⁺ T-cells, total splenocytes from OT-I mice were cultured with SIINFEKL peptide for 48 h. All CD8⁺ T-cells were cultured in RPMI-1640 medium containing 2 mM glutamine, 10% FBS, 25 mM HEPES, pH 7.2, 1% penicillin-streptomycin, 55 μM β-mercaptoethanol and supplemented with or without 20 ng/ml recombinant murine IL-2 (Biolegend, 575404), unless otherwise stated.

Human CD8⁺ T cells were magnetically isolated from blood of healthy donors after gradient centrifugation. Incubation with microbeads conjugated to anti-human CD8 antibodies (Miltenyi 130-097-057) was followed by magnetic bead isolation on a MACS column. Purified human CD8⁺ T cells were activated with αCD3 and αCD28 coated beads (Dynabeads, 111.61D) and expanded in the presence of recombinant human IL-2 (30 Ul/mL, Roche 11011456001).

1.2 Cell Culture

Following activation for 48 h, CD8⁺ T-lymphocytes were cultured in either 21% or 1% oxygen conditions for various times. High glucose DMEM supplemented 10% FBS and 0.8 mg/ml of G418, was used as standard growth medium for RCC4EV, RCC4VHL, 786-OEV, 786-OVHL, B16-OVA and EG7-OVA cells. MEFs and EL-4 cells were cultured in high glucose DMEM supplemented with 10% FBS, 1% penicillin/streptomycin. For low oxygen experiments, cells were transferred into a Ruskinn Sci-tive hypoxia workstation for the indicated amount of time. S-2HG octyl ester and R-2HG-octyl ester was purchased from Toronto Research Chemicals and DCA from Sigma. α-KG-octyl ester was purchased from Caymen. S-2HG and R-2HG free acids were purchased from Sigma

1.3 Cell Counting, Volume and Viability

Cells were counted on an ADAM-MC automated cell counter (NanoEnTek) and viability was assessed by the exclusion of propidium iodide. Cell volume was determined using a Z2 Coulter counter (Beckman Coulter).

1.4 Flow Cytometry and Sorting

Cells were stained and acquired on a Fortessa (BD Biosciences). The following fluorophores and fluorophore-conjugated antibodies were used: anti-CD62L (Biolegend, MEL-14), anti-CD44 (Biolegend, IM7), anti-CD8a (Biolegend, 53-6.7), anti-CD45.1 (Biolegend, A20), anti-CD45.2 (Biolegend, 104), anti-CD127 (Biolegend, A7R34), anti-KLRG1 (Biolegend, 2F1/KLRG1), anti-CD19 (Biolegend, 6D5), anti-Bcl-2 (Biolegend, BCL/10C4), Eomes (eBioscience, Danlimag), PD-1 (Biolegend, 29F.1A12), 4166 (Biolegend, 1765), H-2Kb/SIINFEKL Pentamer-PE (Proimmune, F093-2A), CFSE (Life Technologies), LIVE/DEAD Violet (Life Technologies), PI-AnnexinV (Biolegend, 640928), anti-Bcl-xl-PE (Cell Signalling Technologies, 54H6). For staining of nuclear-located marker Eomes, the Transcription Factor Staining Buffer Set (eBioscience) was used, according to the manufacturer's instructions. For staining of intracellular cytokines, OT-I cells were re-stimulated with SIINFEKL peptide for 4 h with GolgiStop solution (BD). For ¹H-NMR experiments, CD8⁺ T-lymphocytes were sorted from total splenocytes by immunostaining with anti-mouse CD8-AF647 (Biolegend) on a MoFlo (Beckman Coulter). Cells were then activated and cultured as described above. For staining of human T cells, CD8⁺ T cells were incubated at 37 C for 30 minutes in the presence of anti-CCR7 antibody (eBioscience 12-1979-41), followed by staining with anti-CD45RO antibody (Biolegend, UCHL1).

1.5 Generation of Vhl^{-/-} Mouse Embryonic Fibroblasts

Mouse embryonic fibroblasts (MEFs) were isolated from E12.5-14.5 Vhl^{Fl/Fl} embryos. MEFs were then immortalised by stable transfection with the SV40 large T antigen. Fifteen passages later, to perform acute deletions of Vhl, cells of each genotype were transiently (24 h) infected with 100 PFU/cell of adenovirus expressing either eGFP alone, or both Cre recombinase and eGFP (Vector Biolabs). Cell populations were then enriched for eGFP by sorting with on a MoFlo (Beckman Coulter) flow cytometry.

1.6 Determination of Deletion Efficiency by QPCR

To quantify deletion efficiency, gDNA was isolated using a DNeasy Blood & Tissue Kit (Qiagen) followed by real-time PCR. Abundance of the target gene was normalized to Actb gDNA levels.

1.7 Sequencing of Idh1 and Idh2

gDNA was extracted from the following CD8⁺ T-lymphocytes samples: freshly isolated, naïve Hif1a^{Fl/Fl} and Hif1a^{-/-}; expanding Hif1a^{Fl/Fl} and Hif1a^{-/-} after a total of 4 days in 21% oxygen; expanding Hif1a^{Fl/Fl} and Hif1a^{-/-} after 2 days at 21% oxygen and a further 2 days at 1% oxygen (total of 4 days in culture). PCR was performed followed by purification of the product and Sanger sequencing.

1.8 qRT-PCR

RNA was isolated using the RNeasy kit (Qiagen). 1 μg of RNA was used for cDNA synthesis with the First-Strand Synthesis kit (Invitrogen). All samples were run in technical triplicates using a StepOnePlus system (Applied Biosystems) with SYBR green.

1.9 Glucose, lactate, VEGF, IL-2 and IFN-γ Measurements in Media

CD8⁺ T-cells were expanded for 4 days and then treated for 16 h with S-2HG octyl ester or R-2HG octyl ester. Glucose and lactate levels in culture medium were measured with a Dade-Behring Dimension RXL analyser. Changes in metabolite concentrations relative to fresh media were normalized to viable cell counts. VEGF, IL-2 and IFN-γ protein levels in media were determined using the following kits from MesoScale Discovery: K150BMC-2 for VEGF, K15048D-2 for IFN-γ and K152QQD-2 for IL-2. Values were normalized to viable cell counts.

1.10 In Vitro Cytotoxicity Assay

Total splenocytes from OT-I mice were activated with SIINFEKL-peptide for 48 h and expanded for a further 4 days in IL-2 containing medium. OT-I-specific CD8⁺ T-cells were then treated for 24 h with or without 0.5 mM S-2HG-octyl ester or R-2HG-octyl ester and then incubated with target (EG7-OVA, CFSE-low) and control (EL4, CFSE high) cells. Specific lysis was calculated by normalization of the loss in signal of the CFSE-low population relative to the CFSE-high population after co-culture of 4 hours with the vehicle or S-2HG-octyl ester or R-2HG-octyl ester-treated OT-I-specific CD8⁺ T-cells.

1.11 Metabolomics

Metabolomics experiments were performed using GC/MS and LC/MS/MS platforms (Metabolon Inc.) for determination of intracellular metabolites. Samples were normalized using protein concentration measured by the Bradford assay and rescaled to set the median to 1. Missing values were imputed with the minimum value. Analyses performed with MetaboAnalyst 2.0 (Xia et al. 2009; Xia et al. 2012) included unsupervised hierarchical clustering and principal component analysis.

1.12 ¹H-NMR Data Acquisition and Analysis

¹H nuclear magnetic resonance (¹HNMR) spectroscopy was performed with solvent-suppression on a 600 MHz Bruker Avance NMR spectrometer with 4,4-dimethyl-4-silapentane-1-sulfonic acid (DSS) as an internal standard. Electronic RefeREnce To access In vivo Concentration (ERETIC) method⁷ was used to determine the concentration of DSS in each sample. Processing of ¹HNMR spectra included both zero- and first-order phase corrections followed by baseline correction using Chenomx NMR Suite 7.6 (Chenomx Inc.). 2-hydroxyglutarate was identified, based on chemical shift assignment, also using the Chenomx NMR Suite 7.6. Spectral intensities were normalized to the internal standard in each sample.

1.13 Mass Spectrometry

Cells were counted to determine viable cell numbers. 2-3 million viable cells were harvested, washed in ice-cold PBS and extracted in ice-cold 80% methanol. After two-freeze thaw cycles, precipitated proteins were removed by centrifugation at 16,000 g and kept for protein content determination by the Bradford assay. Supernatants were evaporated to dryness and samples were reconstituted in the appropriate buffer for each assay. For the quantification of 2HG, glutamate, fumarate, succinate and malate, cell extracts were dissolved in 0.1% formic acid containing a known amount of deuterated2HG (D₃-2HG) as well as ¹³C-labelled glutamate, malate, fumarate and succinate (all m+2), as internal standards. A calibration curve of stable isotope labelled internal standards was run with every batch of samples to allow for absolute quantification. 10 μl of each sample was injected onto a Sciex 6500 MS mounted with a Hypercarb column (Thermo), 100 mm×2.1 mm, 3 μm particle size, held at 50° C., using an Agilent 1290 system. Mobile phase A consisted of 0.1% formic acid in water. Mobile phase B consisted of 0.1% formic acid in acetonitrile. The gradient profile, with a 0.4 ml/min flow rate, was as follows: 95% A for 1.0 min, 70% A for a further 2.5 min, 5% A for a further 1.5 min. The MS conditions included no splitting, HES ionization with a source temperature of 350° C. and negative polarity. The precursor ions for 2HG, glutamate, succinate, fumarate, malate, D3-2HG, ¹³C-glutamate, ¹³C-succinate, ¹³C-fumarate and ¹³C-malate were 147, 146, 117, 115, 133, 150, 148, 119, 117 and 135 m/z respectively. The product ions for 2HG, glutamate, succinate, fumarate, malate, D3-2HG, ¹³C-glutamate, ¹³C-succinate, ¹³C-fumarate and ¹³C-malate were 129, 128, 73, 71, 115, 132, 130, 74, 72 and 117 m/z respectively. Typical retention times for 2HG, glutamate, succinate, fumarate, malate, D₃-S-2HG, ¹³C-glutamate, ¹³C-succinate, ¹³C-fumarate and ¹³C-malate were 1.65, 0.63, 1.49, 2.91, 1.15, 1.67, 0.67, 1.49, 2.91 and 1.15 min respectively. For the enantioselective determination of 2HG, cell extracts were dissolved in H₂O:MeOH (5:95 v/v) containing 0.3% acetic acid and 0.1% ammonium hydroxide, as well as a known amount of D₃-2HG. A calibration curve of stable isotope labelled internal standards was run with every batch of samples to allow for absolute quantification. 5 μl of each sample was injected onto a Sciex 6500 MS mounted with a Astec CHIROBIOTIC R column, 25 cm×4.6 mm, 5 μm particle size, held at ambient temperature, using an Agilent 1290 system. The mobile phase consisted of H₂O:MeOH (5:95 v/v) containing 0.3% acetic acid and 0.1% ammonium hydroxide. The mobile phase was run isocratically at a flow rate of 1.2 ml/min for 9.6 min. The MS conditions were as above. Typical retention times were 3.71 and 4.33 min for S-2HG and R-S-2HG respectively. All ¹³C tracer studies were performed in medium containing 10% dialysed FBS. RPMI-1640 medium free from glucose or glutamine was prepared so that each substrate pool was entirely labelled whilst the other not. The final concentrations of [U-¹³C₆] glucose or [U-¹³C₅] glutamine were 11 mM and 2 mM respectively. Medium was also supplemented with 25 mM HEPES pH 7.4, 1% penicillin-streptomycin and 55 μM β-mercaptoethanol. [U-¹³C₆] glucose and [U-¹³C₅] glutamine were purchased from Cambridge Isotope Labs. Steady-state labelling was achieved by culturing cells in the presence of tracer for 24 h. The multiple reaction monitoring (MRM) transitions monitored, corresponded to loss of water (−18 m/z). The MRM transitions used for m+0, m+1, m+2, m+3, m+4 and m+5 2HG were 147-129, 148-130, 149-131, 150-132, 151-133 and 152-134 m/z respectively.

1.14 Immunoblotting

Nuclear and cytosolic fractions were prepared from cells with the NE-PER kit (Thermo Scientific) and separated by SDS-PAGE. Proteins were transferred to PVDF membranes and then blocked in 5% milk prepared in phosphate-buffered saline (PBS) plus 0.05% Tween 20. Membranes were then incubated with primary antibodies overnight at 4° C. and horseradish peroxidase (HRP)-conjugated secondary antibodies for 1 h the next day. The following primary antibodies were used at a dilution of 1:1000: HIF1α (Novus), HIF2α (Novus), PDH-E1α (Abcam), PDH-E1α pS232 (Calbiochem), LaminB1 (Abcam), HDAC1 (Abcam), β-tubulin (Abcam), 4E-BP1 (Cell Signalling), phospho-4E-BP1 (S65) (Cell Signalling), phospho-4E-BP1 (T37/T46) (Cell Signalling) S6K (Cell Signalling), phopho-S6K (T389) (Cell Signalling), phopho-S6K (S371) (Cell Signalling).

1.15 Protein Quantification Protein quantification was performed using the DC-Protein Assay (BioRad) according to the manufacturer's instructions. Absorbance at 595 nm was measured and samples were quantified against a standard curve constructed using known concentrations of bovine serum albumin (BSA).

1.16 In Vivo Memory Recall Experiment

Total splenocytes from OT-1 CD45.1⁺ mice were activated with 1000 nM SIINFEKL peptide in the presence of IL-2 and vehicle, 0.5 mM S-2HG-octyl ester or 0.5 mM R-2HG-octyl ester for 7 days. 1 million CD45⁺CD8⁺ cells were then injected intravenously into C57/B6 wild type CD45.2⁺ host mice. 30 days later, host mice were vaccinated i.p. with SIINFEKL-loaded dendritic cells. 7 days later, spleens were harvested from host mice and the presence of CD45.1⁺CD8⁺ and Kb/SIINFEKL Pentamer+cells was determined by flow cytometry. Absolute numbers of cells were determined with the use of counting beads (CountBright, Life Technologies). Dendritic cells were prepared from bone marrow extracted from wild type C57/B6 mice. Bone marrow derived cells were cultured in non-TC treated petri dishes in RPMI-1640 medium containing 2 mM glutamine, 10% FBS, 25 mM HEPES, pH 7.2, 1% penicillin-streptomycin, 55 μM 3-mercaptoethanol supplemented 20 ng/ml mGM-CSF (R&D Systems). After 8 days of culture, dendritic cells were activated with 1 μg/ml LPS (Sigma) for 24 h. The maturation of dendritic cells was confirmed by flow cytometry using the following markers: MHC class II-APC, CD11b-AF488, CD11c-AF488 and CD86-PE/Cy7 (Biolegend). Mature dendritic cells were then loaded with 2 μM SIINFEKL peptide at 37° C. for 1 hour. After peptide loading, dendritic cells were detached with 3 mM EDTA in PBS for 5-10 min at 37° C. and washed with PBS. For vaccination, 1 million peptide-loaded dendritic cells were injected i.p. per mouse in 100 μl PBS.

1.17 Animal Models

Mice were bred and housed in specific pathogen-free conditions in accordance with the UK Home Office and the University of Cambridge. Deletion of the following loxP-flanked alleles in CD8⁺ T-lymphocytes was achieved via breeding with dLck mice²³: Hif1a^{Fl/F l35}, vhl^{Fl/Fl 36} and Epas1^{FlFl 37}. All mice were backcrossed over ten generations to the C57/B6 background. OT-I mice³⁸ containing transgenic inserts for mouse TCR-Vα2 and TCR-Vβ5 genes that recognise ovalbumin residues 257-264 (SIINFEKL) in the context of H-2K^{b} were crossed with CD45.1 mice³⁸. Randomization and blinding were introduced for all mouse experiments.

1.18 Statistical Analysis

Statistical analyses were performed in GraphPad Prism 6 software. Pairwise comparisons were assessed using an unpaired Student's t-test with Welch's correction when appropriate. Multiple comparisons were assessed with one-way ANOVA, including Bonferroni's correction for multiple testing. Grouped data were assessed by two-way ANOVA, including Bonferroni's correction, to adjust for multiple comparisons. Error bars are shown as s.d. as indicated in figure legends.

1.19 shRNA and Overexpression

For shRNA-mediated knockdown experiments, shRNAs were cloned into pMKO.1GFP (pMKO.1 GFP was a gift from William Hahn, Addgene plasmid #10676). shRNA-mediated knockdown of target genes was achieved by transduction with retrovirus expressing L2hgdh shRNA or scrambled shRNA. pMKO.1GFP vectors containing the shRNA of interest were transfected into Phoenix cells with pCL-Eco (pCL-Eco was a gift from Inder Verma (Addgene plasmid #12371)) using Lipofectamine 2000 (Thermo Fisher). Viral supernatants were collected 48 h later. Primary CD8⁺ T-cells were transduced with viral supernatants using rectronectin (Takara Clontech) according to the manufacturer's instructions. Experiments were conducted at time points indicated in the figure legends and GFP was used as a selection marker. Knockdown of L2hdgh mRNA was confirmed by qRT-PCR. For expression of L2hgdh, the vector SFG.wtCNb_opt.IRES.eGFP (gift from Martin Pule (Addgene plasmid #22492)) was used to generate an Empty Vector control, by replacing the insert with a multiple cloning site. Murine DNA sequences encoding C-terminal FLAG tagged L2hgdh were cloned into Empty Vector. Retrovirus encoding each enzyme was produced as for shRNA experiments and primary CD8⁺ T-cells were transduced as before. Cells were placed in 21% or 1% oxygen the day after transduction and GFP⁺ cells were assessed by flow cytometry 7 days later.

1.20 In Vivo Persistence Experiments

Total splenocytes from OT-I CD45.1⁺ mice were activated with 1000 nM SIINFEKL peptide in the presence of IL-2 and vehicle or 300 μM S-2HG-octyl ester. After 48 h CD8⁺ T-cells were purified by negative selection and cultured for 7 days in the presence of IL-2 and vehicle or 300 μM S-2HG-octyl ester. 1 million CD45.1⁺CD8⁺ cells were then injected intravenously into C57BL/6J wild type CD45.2.2 host mice. 30 days later, host mice were sacrificed to assess the persistence of transferred cells in the spleen. Absolute numbers of cells were determined with the use of counting beads (CountBright, Life Technologies).

1.21 Homeostatic Proliferation

Total splenocytes from OT-I CD45.1/CD45.1 and OT-I CD45.1/CD45.2 mice were activated with 1000 nM SIINFEKL peptide in the presence of IL-2 and vehicle or 300 μM S-2HG-octyl ester. After 48 h CD8⁺ T-cells were purified by negative selection and cultured for a further 7 days in the presence of IL-2 and vehicle or 300 μM S-2HG-octyl ester. Vehicle and S-2HG-treated cells were then mixed 1:1 and labelled with CFSE, followed by intravenous injection into sub-lethally irradiated CD45.2/CD45.2 hosts. 7 days later, spleens were harvested and analysed by flow cytometry. Absolute numbers of cells were determined with the use of counting beads (CountBright, Life Technologies)

1.22 Adoptive Cellular Immunotherapy Experiments

For adoptive cell therapy experiments, OT-I CD8⁺ T-lymphocytes were activated with 1000 nM SIINFEKL peptide in the presence of IL-2 and vehicle or 300 μM S-2HG-octyl ester. After 48 h CD8⁺ T-cells were purified by negative selection and cultured for a further 7 days in the presence of IL-2 and vehicle or 300 μM S-2HG-octyl ester. OT-I cells were transferred intravenously into tumour-bearing C57BL/6J lymphodepleted or lymphoreplete mice with 9-12 day established EG7-OVA tumours. Lymphodepletion was achieved with 5 Gy total body irradiation before adoptive transfer of OT-I CD8⁺ T-lymphocytes.

1.23 Experiments with Human CD8⁺ T Cells

For experiments with human CD8⁺ T cells, cells were magnetically isolated, activated and plated at a concentration of 1×10⁶ cells per mL in the presence of 30 UI/mL of recombinant human IL-2. CD8⁺ T cells were expanded for 14 days in the presence of 600 μM S-2HG-octyl ester, 800 μM S-2HG-octyl ester, or vehicle (control). On day 14, the surface expression of CCR7 and CD45RO on alive CD8⁺ cells was measured by flow cytometry.

2. Results

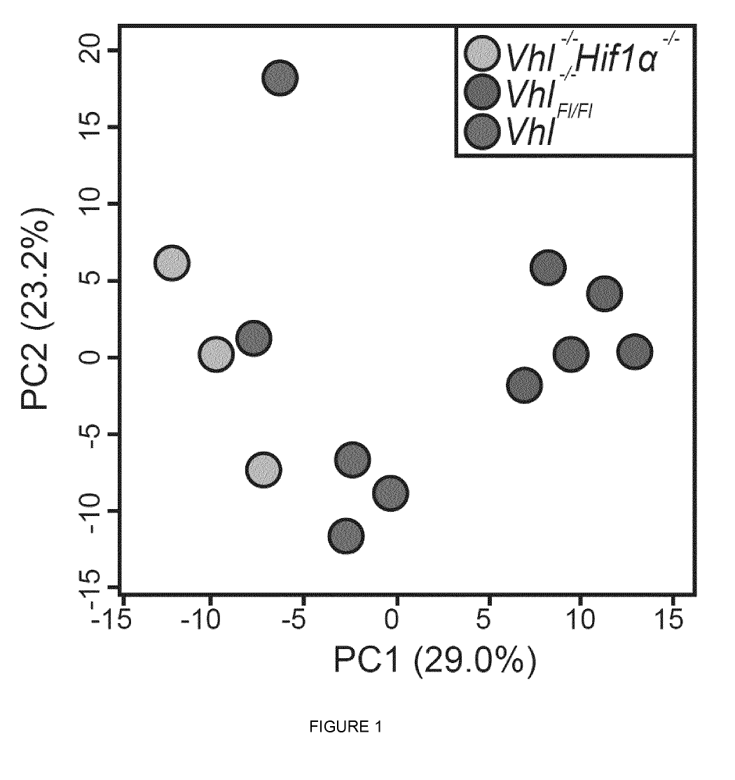

We used unbiased metabolomic profiling to identify metabolites that significantly differ between CD8⁺ T-lymphocytes with low (von Hippel-Lindau, Vh^{Fl/Fl}) and high (Vhl^{-/-}) HIF signalling, as well as HIF-1α-VHL double knockouts (Hif1α^{-/-}Vhl^{-/-}) to control for a specific contribution of HIF-1α to these changes. Unsupervised clustering and principal component analysis (FIG. 1) indicate separation of Vhl^{-/-} CD8⁺ T-lymphocytes from Vhl^{Fl/Fl} (WT) cells. Hif1α^{-/-}Vhl^{-/-} clusters together with this WT control, indicating that HIF-1α mediates most of the metabolic changes following Vhl deletion (FIG. 1). Glycolysis is a critical metabolic pathway for sustaining CD8⁺ T-cell effector function¹⁶ and these data indicate that VHL negatively regulates this via suppression of HIF-1α, with effects being most pronounced on late glycolytic intermediates.

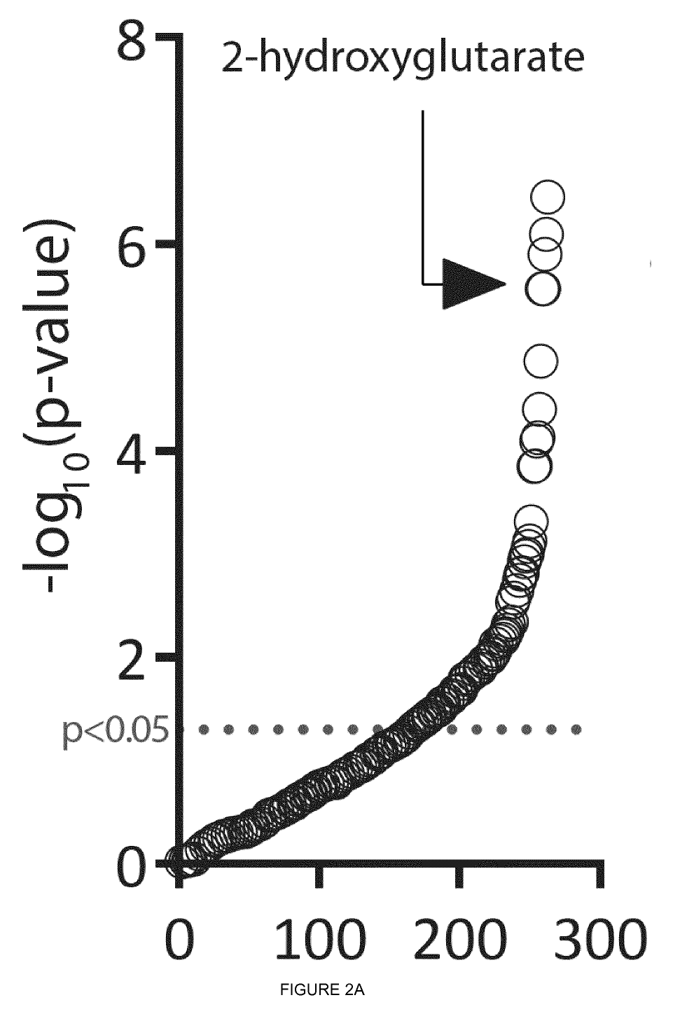

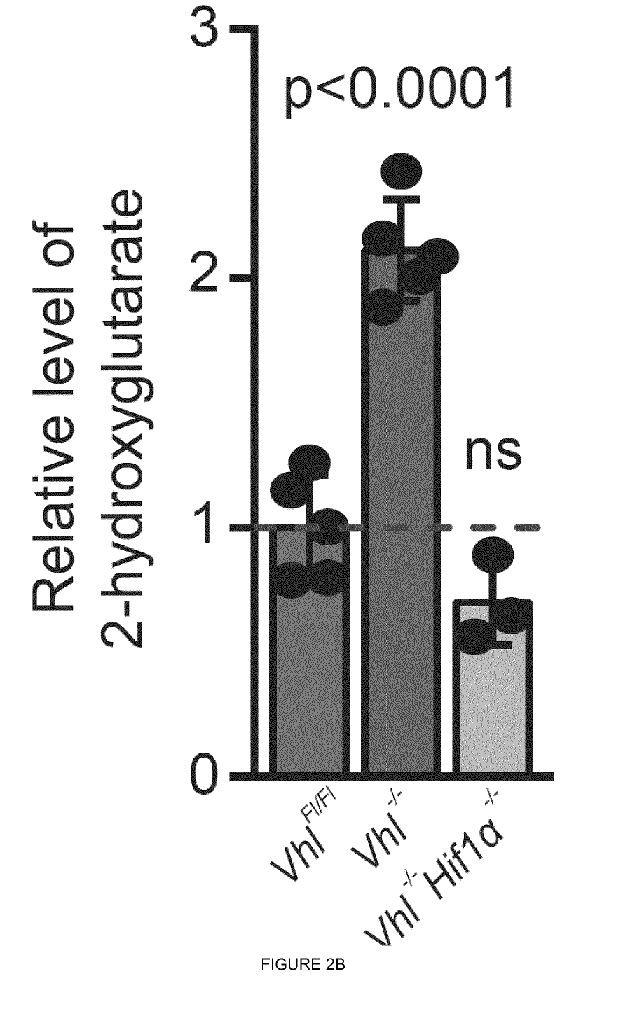

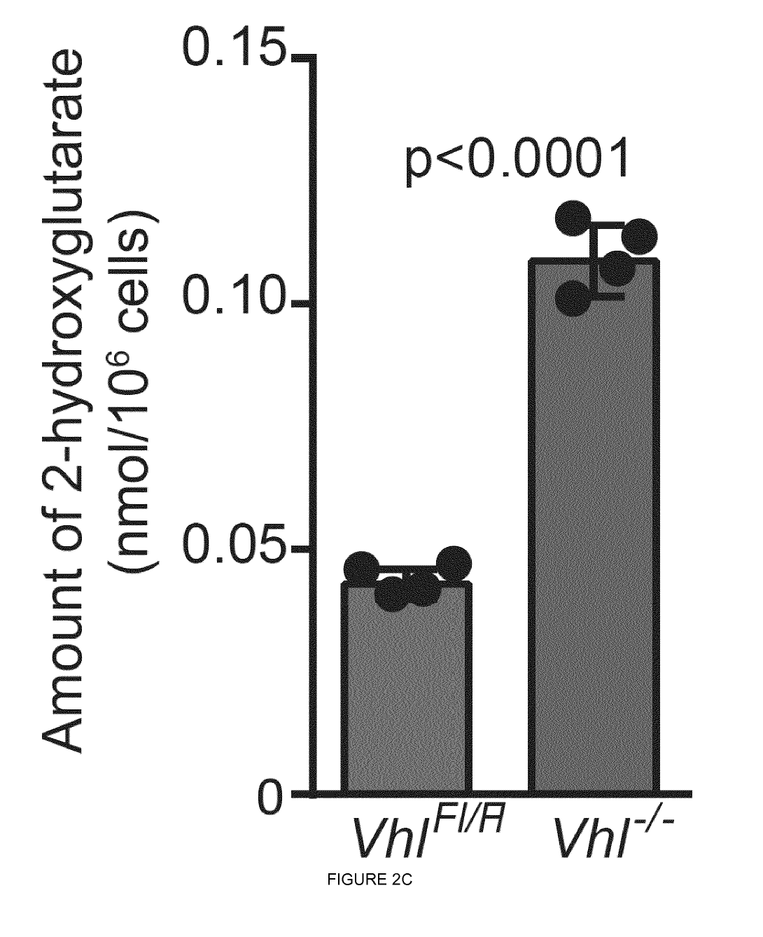

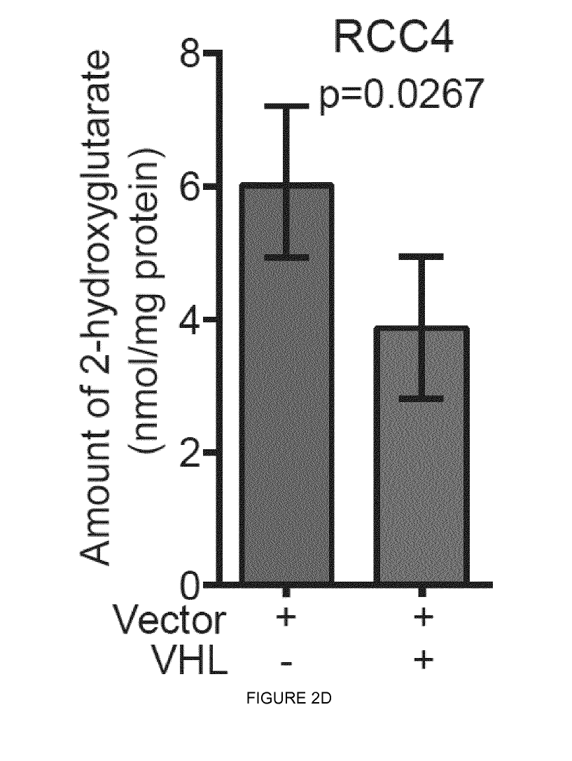

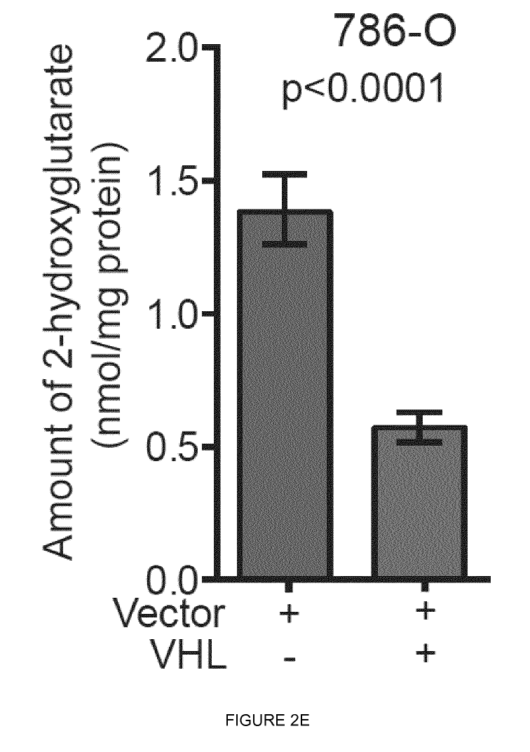

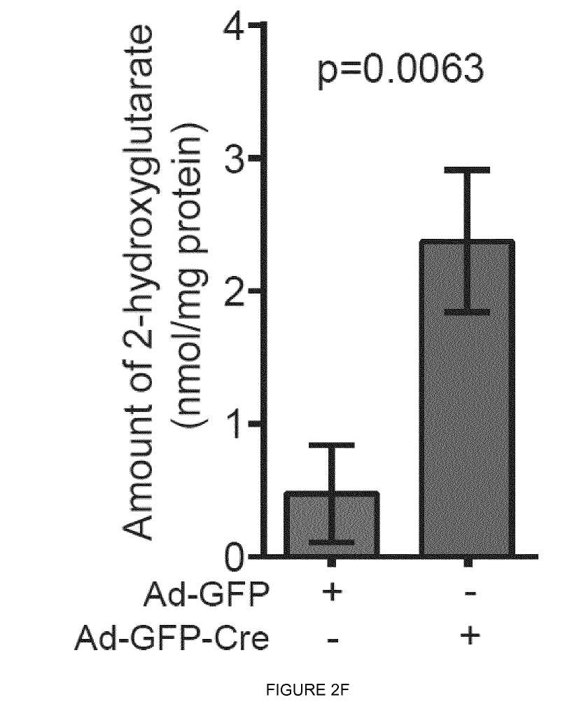

With respect to the tricarboxylic acid (TCA) cycle and related metabolites, we find that VHL loss overall suppresses late, whilst increasing early intermediates. Strikingly, 2HG ranks as one of the most significantly enriched metabolites in Vhl^{-/-} CD8⁺ T-lymphocytes (FIG. 2A). Furthermore, levels of 2HG in Hif1α^{-/-}Vhl^{-/-} versus Vhl^{Fl/Fl} CD8⁺ T-lymphocytes are no different (FIG. 2B), suggesting increases in 2HG are secondary to activation of HIF-1α when VHL is removed. We validated 2HG levels in Vhl^{-/-} CD8⁺ T-lymphocytes by quantitative liquid chromatography-tandem mass spectrometry (LC-MS/MS) (FIG. 2C). We also see that 2HG is elevated in VHL-null RCC4 cells compared to isogenic controls stably expressing VHL (FIG. 2D), further demonstrating the role of VHL in this elevation. Interestingly, 2HG is also elevated in 786-O renal cell carcinoma cells that exclusively express HIF-2α (FIG. 2E). Mouse embryonic fibroblasts (MEFs) isolated from Vhl^{Fl/Fl} mice, where we acutely deleted Vhl by infection with a cre-expressing adenovirus, also display significantly elevated 2HG levels (FIG. 2F). Taken together, our data indicate that the VHL-HIF axis regulates 2HG levels, and that constitutive HIF-1α signalling likely underlies this effect in Vhl-null CD8⁺ T-lymphocytes.

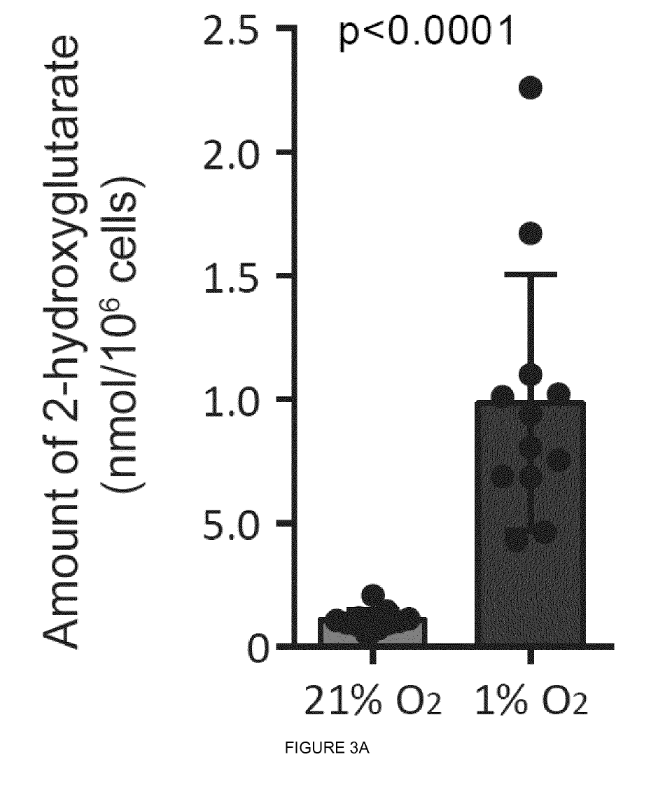

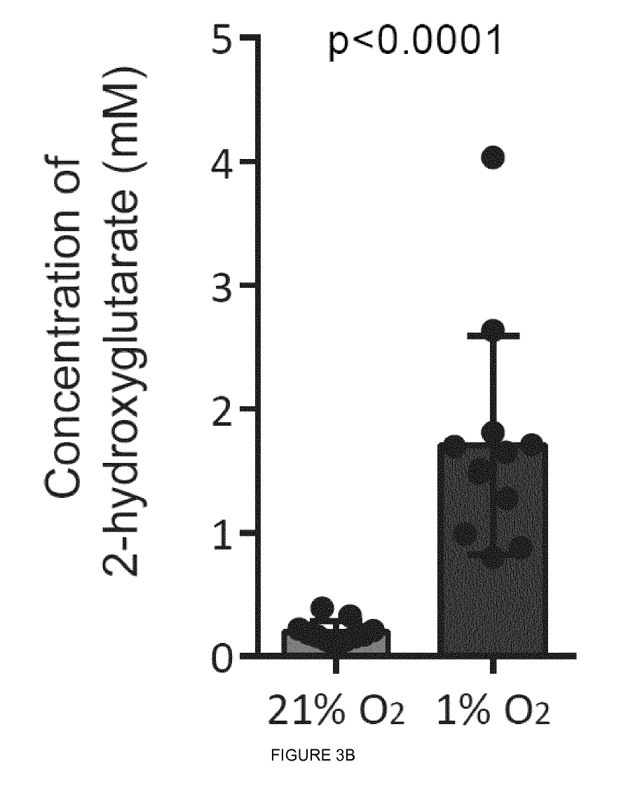

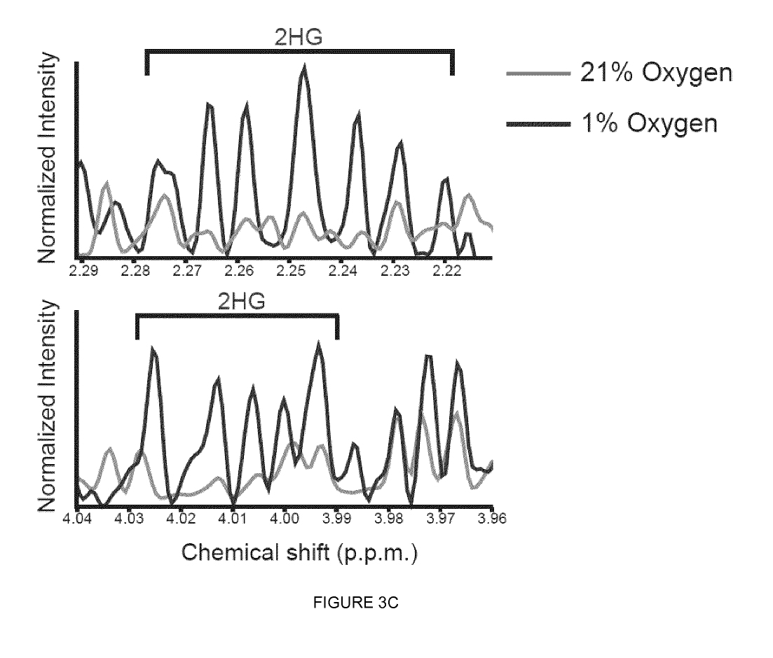

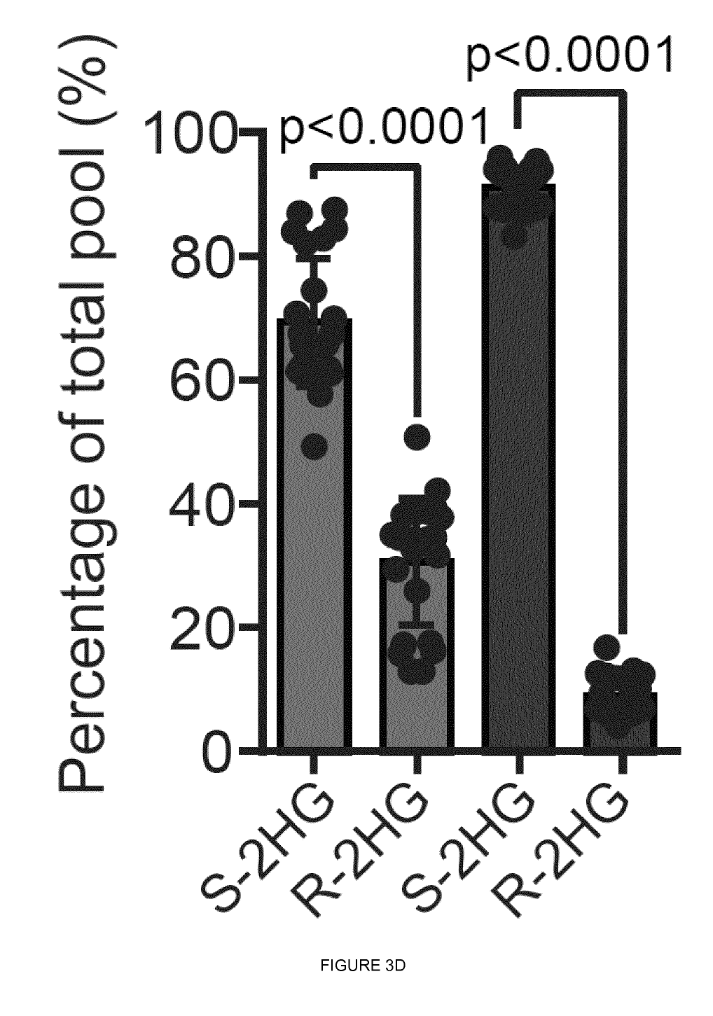

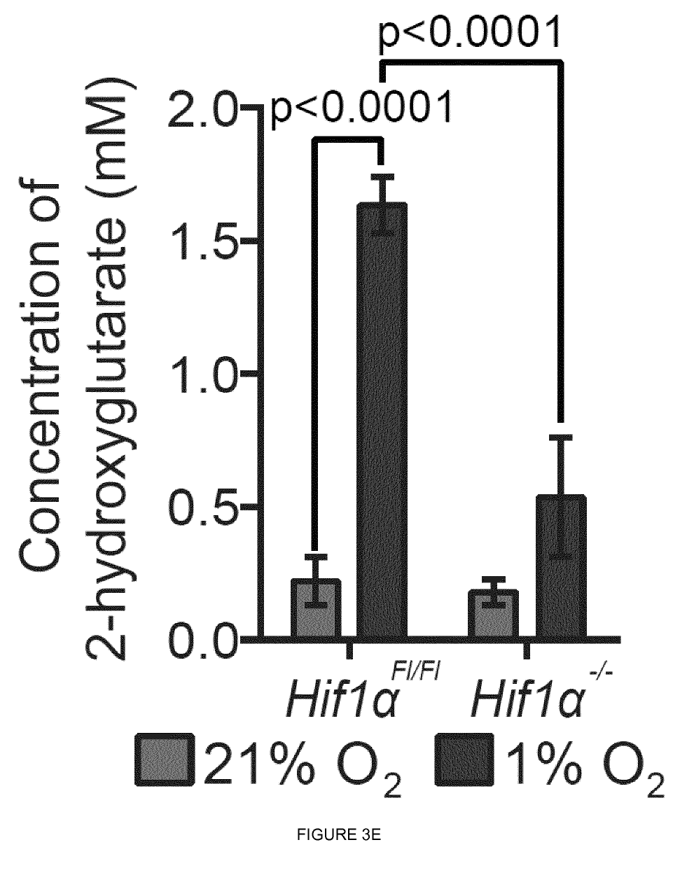

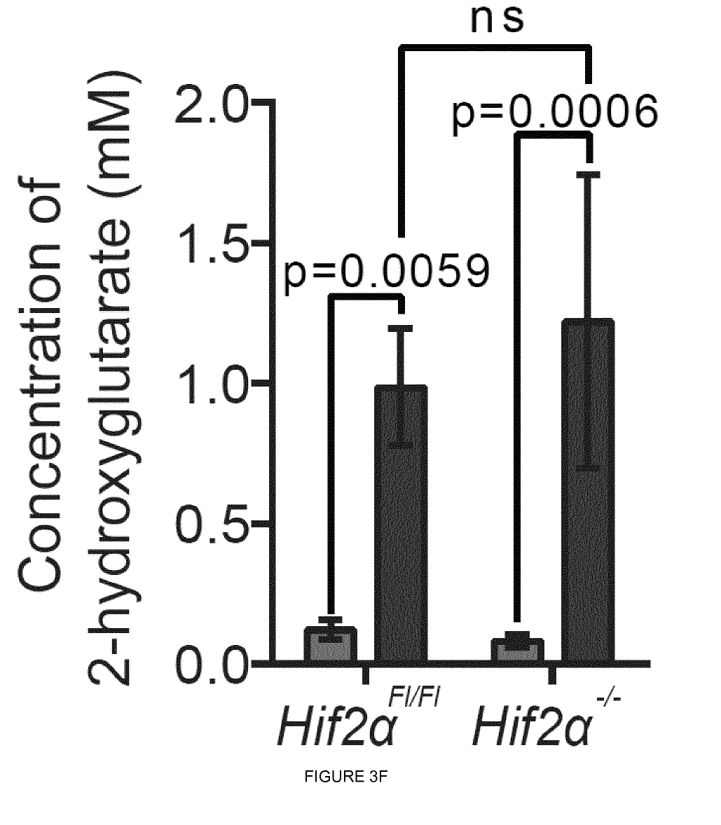

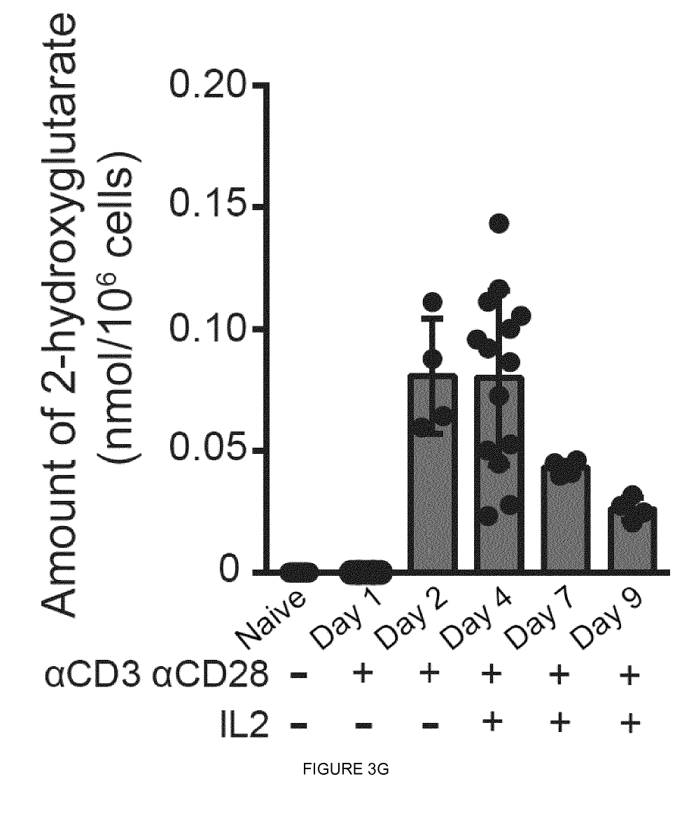

R-2HG is produced by oncogenic IDH1 and IDH2 mutations in many different cancers^{17,18}. However, accumulation of S-2HG occurs in cells with ostensibly wild type IDH1/2 exposed to hypoxia^{9,10}, and those with mitochondrial dysfunction^{19,20,} as well as in renal cell cancer²¹. In activated wild type CD8⁺ T-lymphocytes cultured ex vivo in 21% oxygen, the intracellular concentration of 2HG is 190 μM±30 μM (FIGS. 3A and B). In 1% oxygen, the level of 2HG is markedly elevated (FIGS. 3A and C). Strikingly, the mean intracellular concentration of 2HG is 1.71 mM±0.25 mM in hypoxia (FIG. 3B). Given such high levels of 2HG, we sequenced the conserved active site arginines²² of both Idh1 and Idh2, to preclude the unlikely possibility that expanding primary CD8⁺ T-lymphocytes in hypoxia gives rise to mutations known to cause R-2HG production in humans¹⁷. Consistent with wild type Idh1/2, resolving the S- and R-enantiomers of 2HG, indicates that S-2HG accounts for close to 60% of the total 2HG pool in normoxic activated CD8⁺ primary T-lymphocytes, and this increases to 90% in hypoxia (FIG. 3D).

Since marked S-2HG elevation in CD8⁺ T-lymphocytes occurs in hypoxia, and is dependent on the presence of HIF-1α following VHL loss, we reasoned that HIF signalling regulates the hypoxic accumulation of 2HG in these cells. To test this, we generated mice harbouring loxP-flanked Hif1α or Epas1 (herein referred to as HIF-2a) alleles with cre recombinase expressed under the distal Lck promoter, for deletion of Hif1α or Hif2α in CD8⁺ T-lymphocytes²³. Deletion efficiencies for both Hif1α and Hif2α are over 98% in CD8⁺ T-lymphocytes expanded ex vivo at both 21% and 1% oxygen, with no difference in viability observed at the time points studied. After expansion for a total of 4 days, with the latter 2 days at either 21% or 1% oxygen, we find that the high intracellular concentration of 2HG is maintained in Hif2α^{-/-} but not Hif1α^{-/-} CD8⁺ T-lymphocytes in hypoxia (FIGS. 3E and F). The same holds true when the data are normalized to either viable cells or protein content. We next sought to determine 2HG levels over a time course of normoxic activation of CD8⁺ T-lymphocytes. 2HG in freshly isolated naïve CD8⁺ T-lymphocytes is undetectable, whereas its levels are highly elevated after activation with αCD3 and αCD28 antibodies; this occurs by day 2 of culture with maximal elevation extending to day 4 (FIG. 3G). 2HG levels are then reduced by days 7 and 9 days after activation (FIG. 3G); this indicates temporal regulation of the production of this metabolite following T-cell receptor triggering.

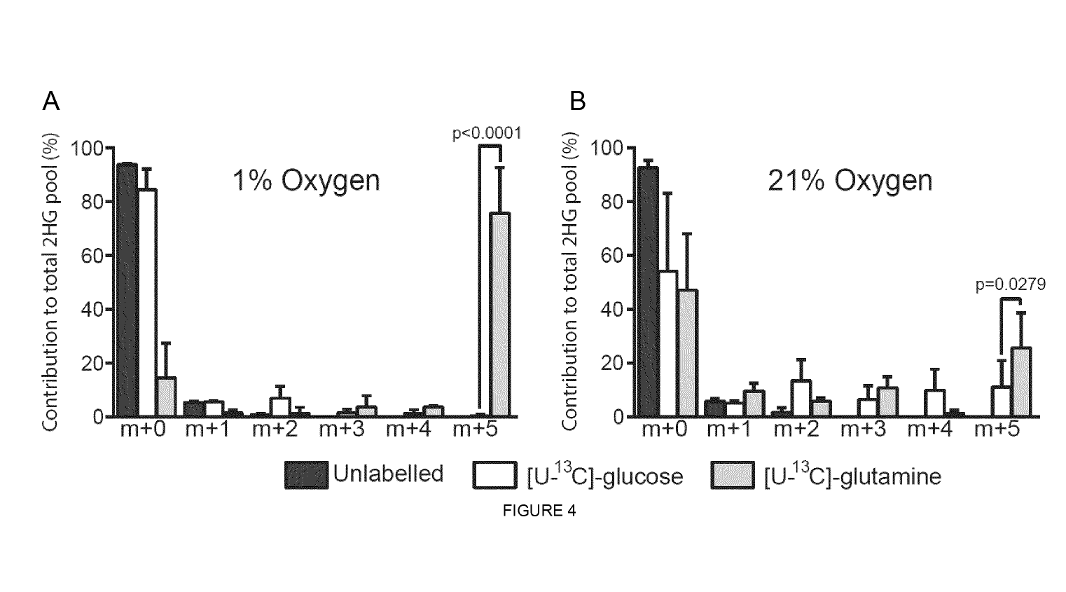

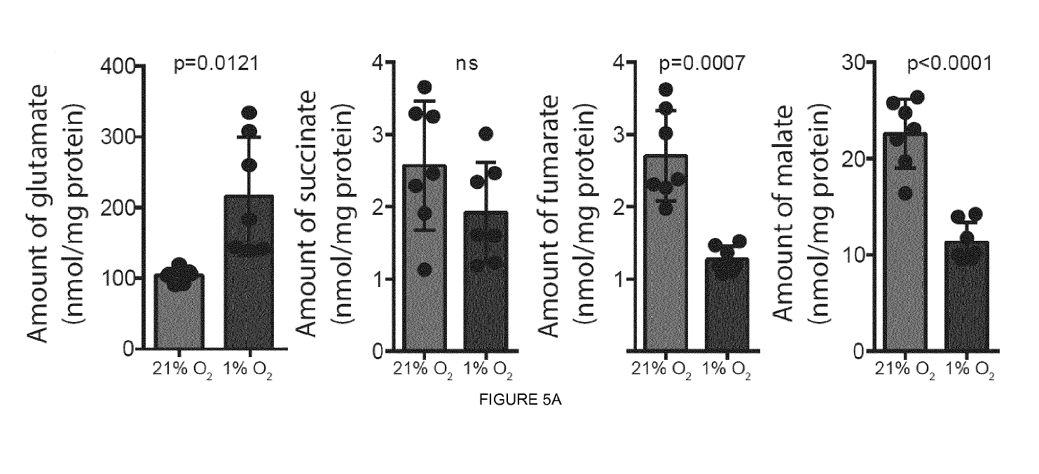

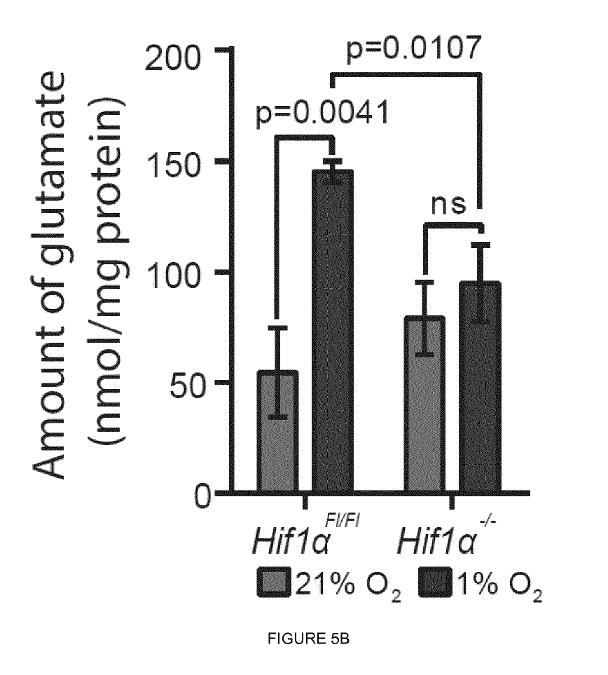

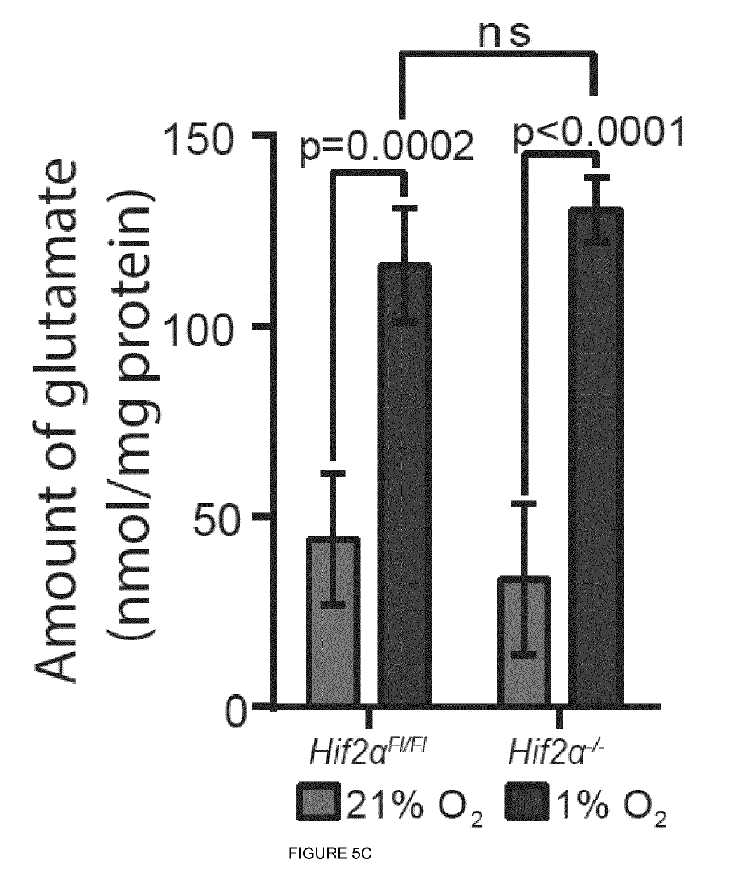

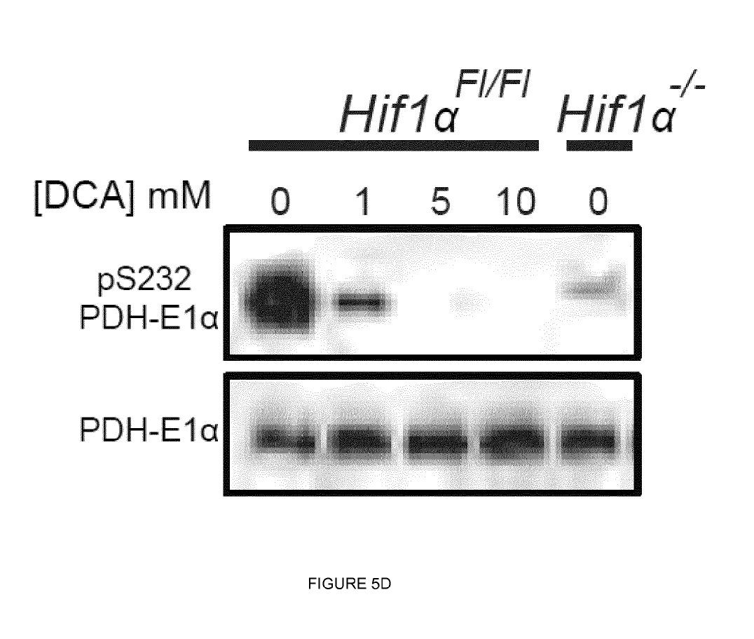

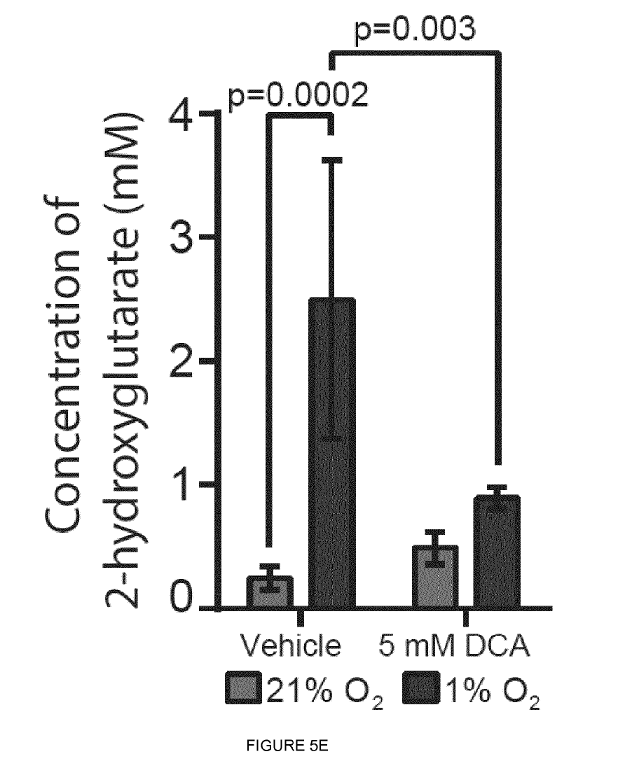

We next sought to determine the metabolic route by which HIF-1α promotes hypoxia-induced production of 2HG. The expression levels of enzymes involved in central carbon metabolism indicate a clear induction of glycolysis and suppression of the TCA cycle. Surprisingly, glutaminase 2 (G1s2) is also induced by hypoxia. Recent reports implicate lactate and malate dehydrogenases (LDHA and MDH1/2), as well as decreases in the dehydrogenase responsible for conversion of S-2HG into 2-oxoglutarate (L2HGDH)^{9,10}, as potential sources of S-2HG in hypoxia. In primary Hif1α^{-/-} CD8⁺ T-lymphocytes, the hypoxic expression of these enzymes suggests that accumulation of S-2HG, via MDH1/2, is unlikely. Furthermore, L2HGDH increases seen in Hif1α^{-/-} CD8⁺ T-lymphocytes are marginal, but might contribute to lower S-2HG levels and may indicate HIF-1α-mediated repression of L2HGDH. However, as expected, HIF-1α-dependent increases in pyruvate dehydrogenase kinase 1 (Pdk1) and Ldha are prominent in hypoxia; interestingly glutaminase 2 (G1s2), but not GIs, shows an identical HIF-1α dependency, implicating glutamine metabolism in hypoxic S-2HG production. Indeed, using U-¹³C-glucose or U-¹³C-glutamine tracers, we find that glutamine is the source of 2HG in 1% oxygen⁹ (FIG. 4A); the m+5 isotopologue dominates, indicating direct conversion of glutamine-derived 2-oxoglutarate to S-2HG¹⁹, possibly via promiscuous LDHA activity⁹. There is a similar contribution to 2HG from both glucose and glutamine in 21% oxygen (FIG. 4B). The absolute succinate pool does not change, however, while the fumarate and malate pools decrease in hypoxia; interestingly, the glutamate pool increases (FIG. 5A). This is also apparent in Vhl^{-/-} CD8⁺ lymphocytes and the hypoxic induction of intracellular glutamate depends on HIF-1α (FIG. 5B) but not HIF-2a (FIG. 5C). Activation of PDK1, with subsequent phosphorylation of pyruvate dehydrogenase (PDH), is a critical point of regulation of reductive glutamine metabolism, driving glutaminolysis^{24,25}. Hence, inhibition of PDK1 should abrogate hypoxia-induced HIF-1α-dependent S-2HG accumulation. Indeed, pharmacological inhibition of PDK1 in wild-type primary CD8⁺ T-lymphocytes with dichloroacetate (DCA) vastly reduces phosphorylation of pyruvate dehydrogenase (PDH) (FIG. 5D), and decreases S-2HG levels in hypoxia (FIG. 5E). Inhibition of PDK1 activity also impedes hypoxia-induced increases in the glutamate pool (FIG. 5F). Together, these data encourage the notion that reorganisation of central carbon metabolism in hypoxic CD8⁺ T-lymphocytes by the HIF-1α-PDK1 axis generates glutamine-derived 2-oxoglutarate, which is then converted by downstream enzymes to S-2HG.

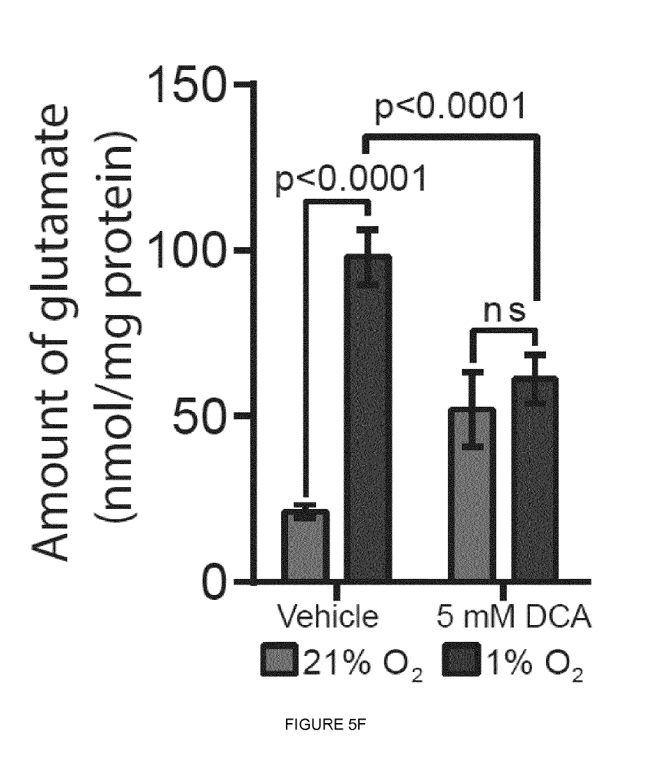

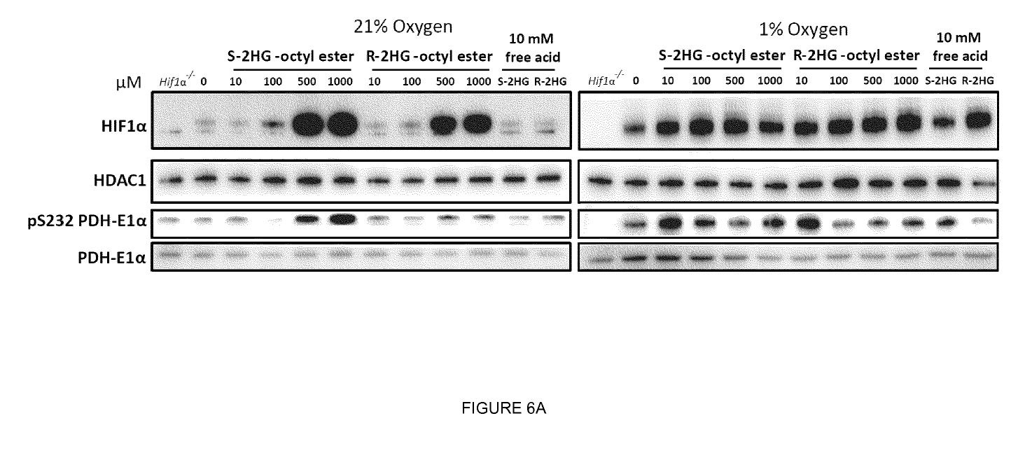

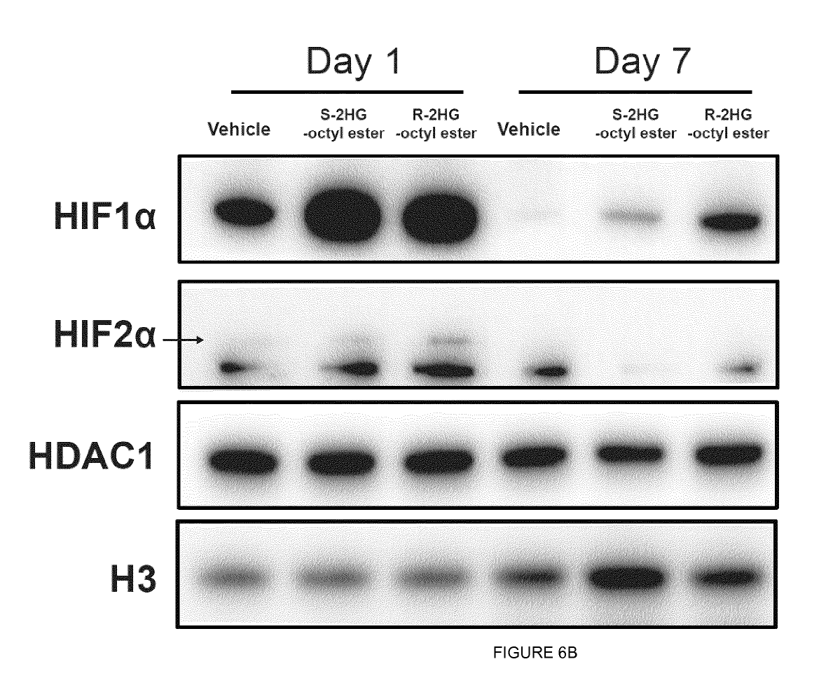

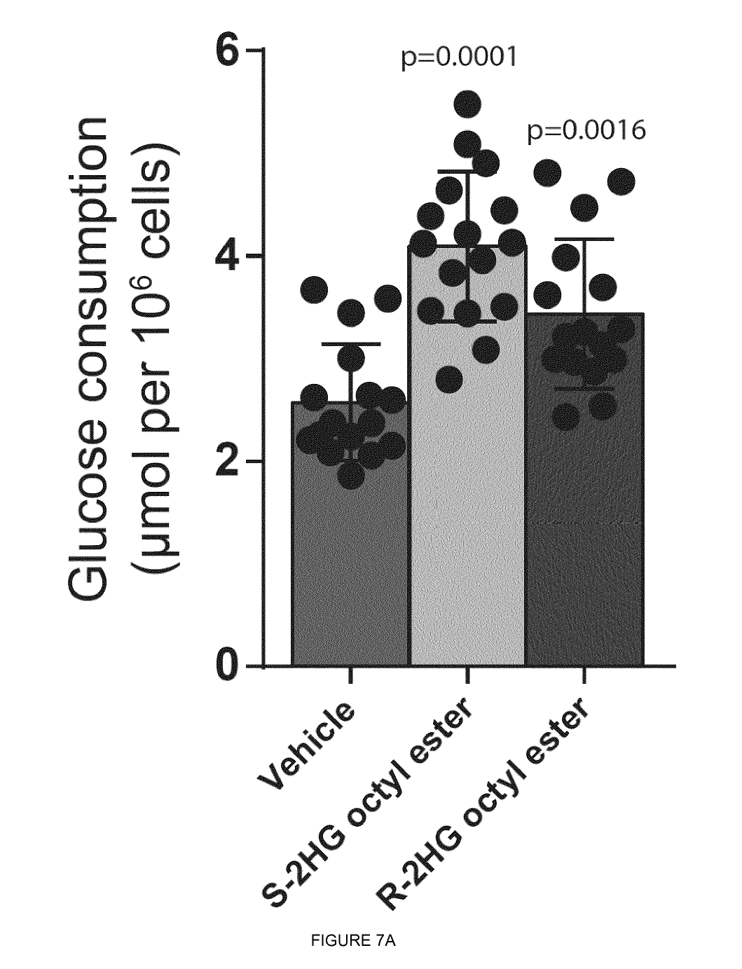

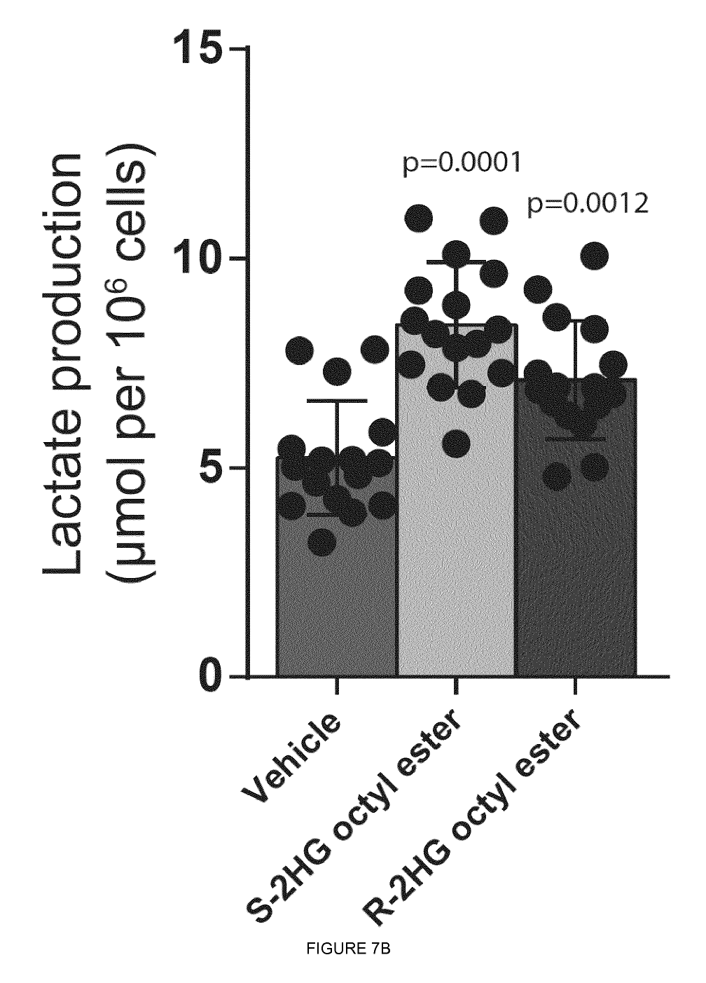

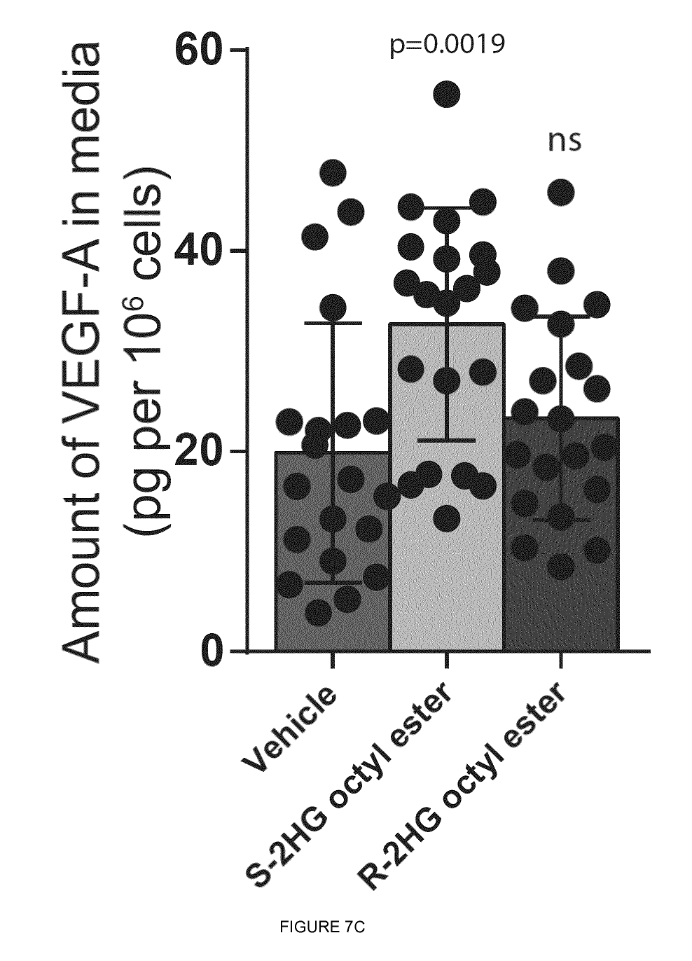

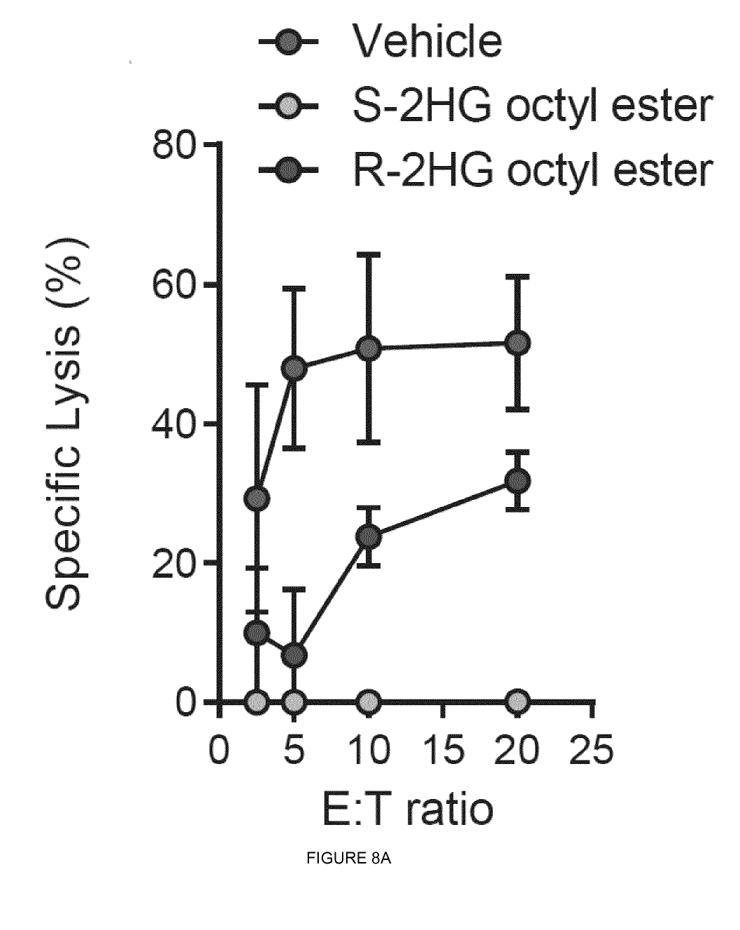

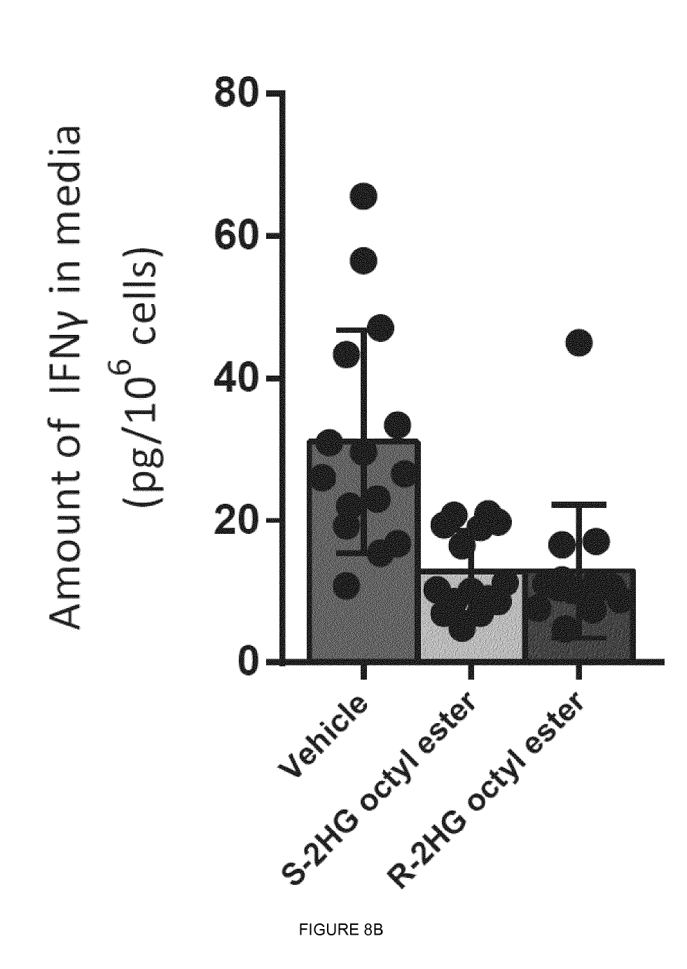

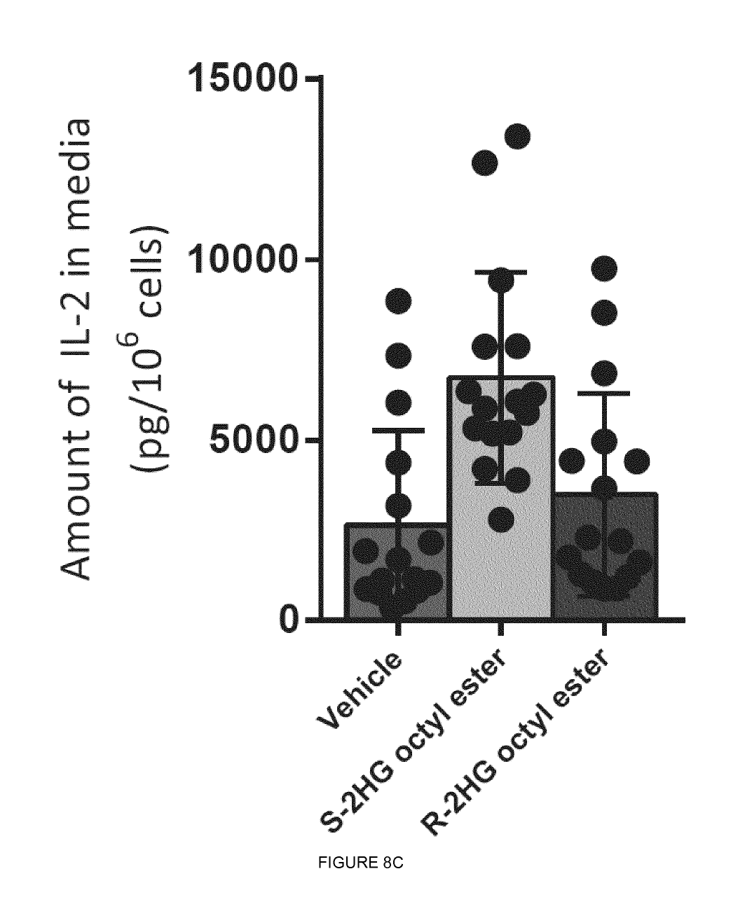

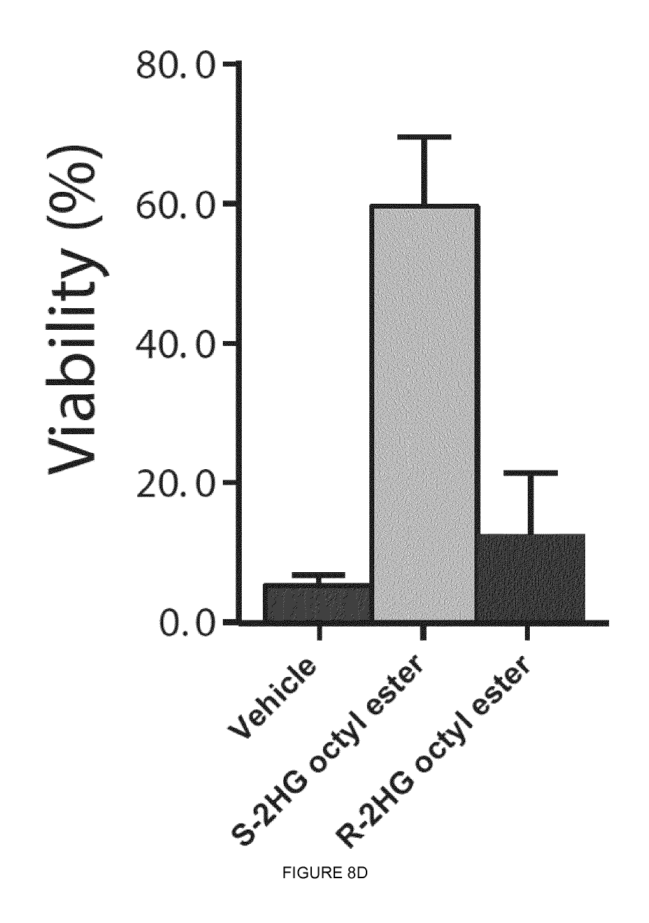

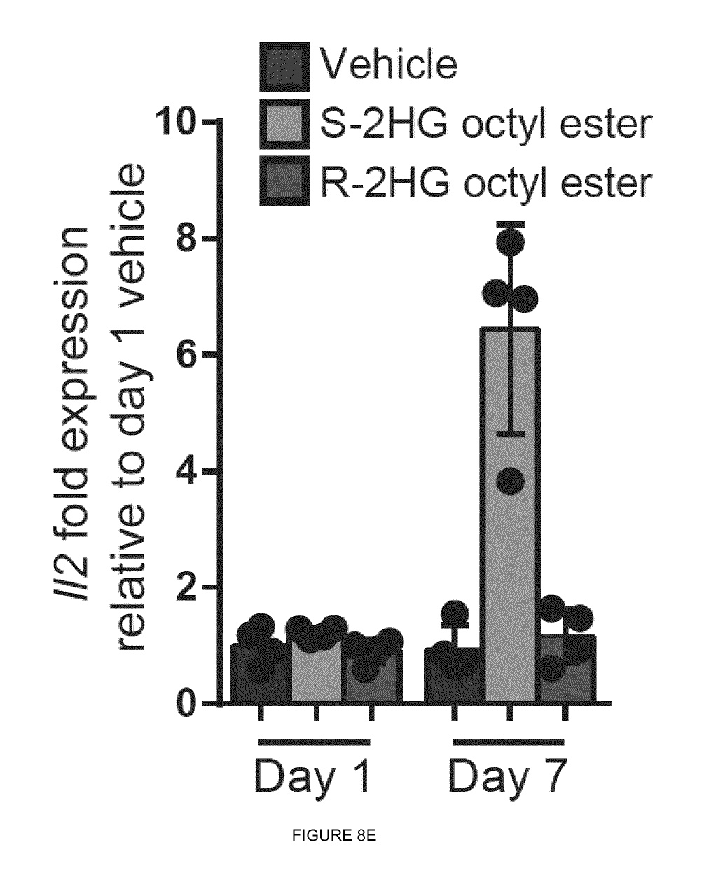

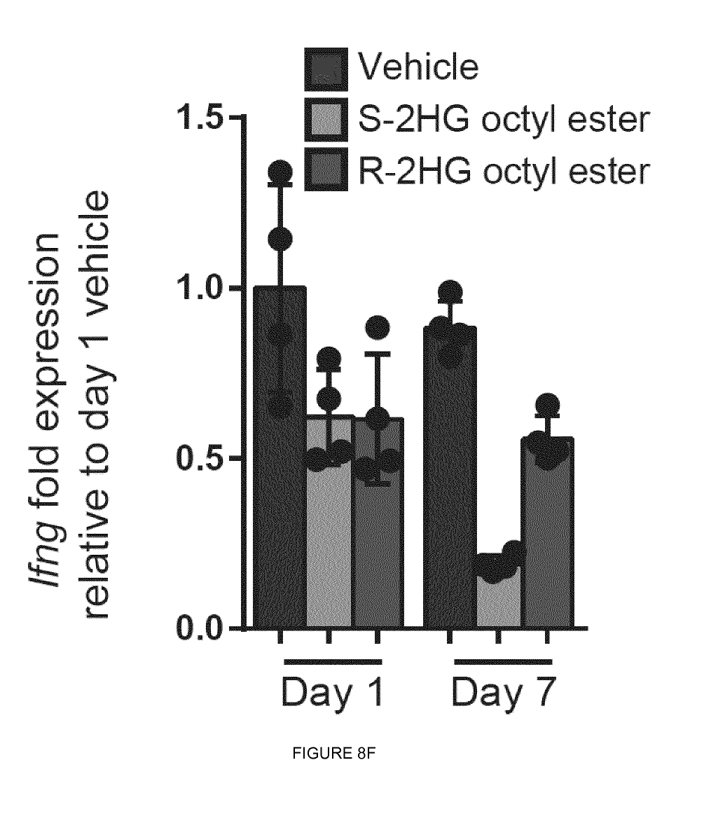

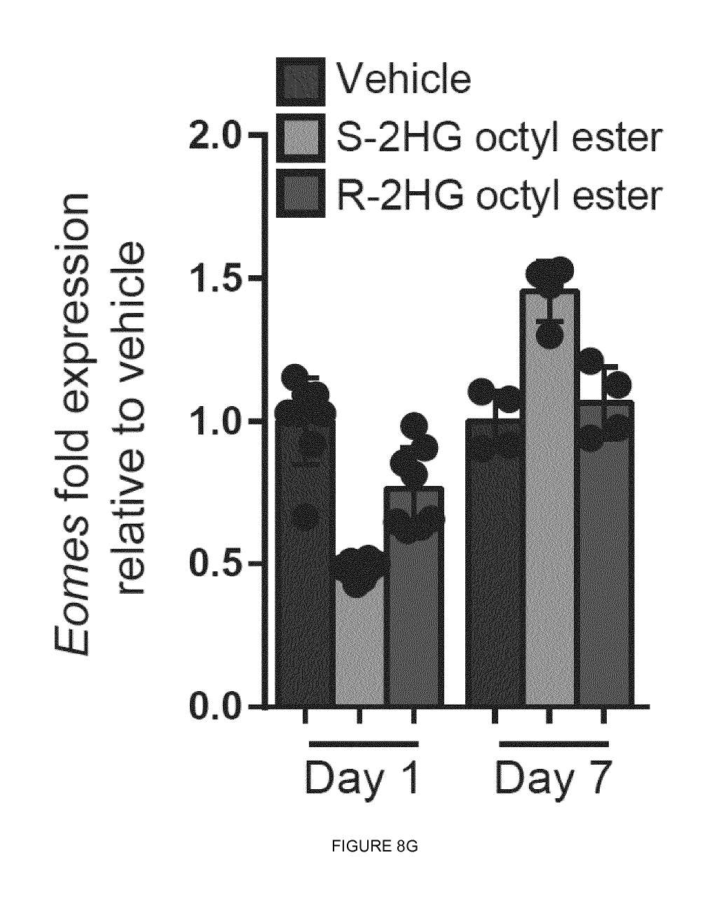

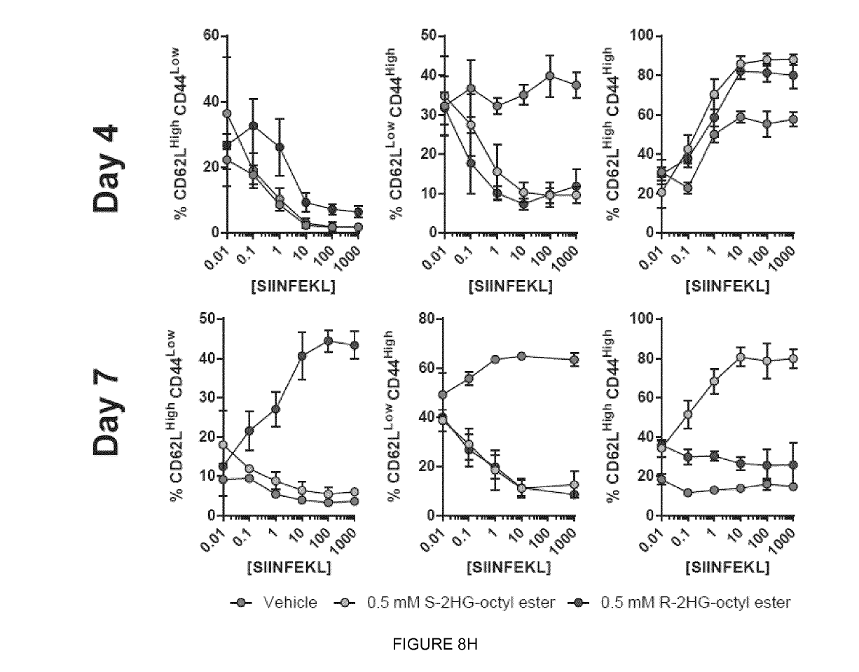

S-2HG is reported to be a potent inhibitor of 2-oxoglutarate-dependent prolyl hydroxylases that hydroxylate HIF-1α for VHL-dependent degradation^{3,4}. Consistent with this, we find HIF-1a is stabilized in CD8⁺ T-lymphocytes by treatment with cell permeable S-2HG-octyl ester in a concentration-dependent manner (FIG. 6A). The same is observed with R-2HG-octyl ester (FIG. 6A), but not with the free acid forms of both molecules which are cell impermeable (FIG. 6A). There is also an increase in the abundance of HIF-2a protein (FIG. 6B). At least for HIF-1α, this increase in abundance is seen even at 7 days of continuous treatment (FIG. 6B). Additionally, there is increased phosphorylation of PDH-E1a (FIG. 6A), elevated glucose uptake (FIG. 7A), lactate secretion (FIG. 7B) and VEGF production (FIG. 7C). Many of these changes are associated with CD8⁺ T-cell effector function^{11,16}; however, and unexpectedly, the lytic ability of antigen specific OT-I CD8⁺ T-cells treated with S-2HG-octyl ester or R-2HG-octyl ester is markedly lower (FIG. 8A). This is associated with decreased secretion of interferon-γ (IFN-γ) (FIG. 8B) and increased production of interleukin-2 (IL-2) (FIG. 8C). There is also an elevated ability to survive in the absence of IL-2 supplementation (FIG. 8D), possibly reflecting an autocrine pro-survival pathway. These effects are mediated at the transcriptional level after both transient and prolonged treatment with S-2HG-octyl ester or R-2HG-octyl ester (FIG. 8E, 8F). These effects are more pronounced with S-2HG-octyl ester treatment than with R-2HG-octyl ester treatment.

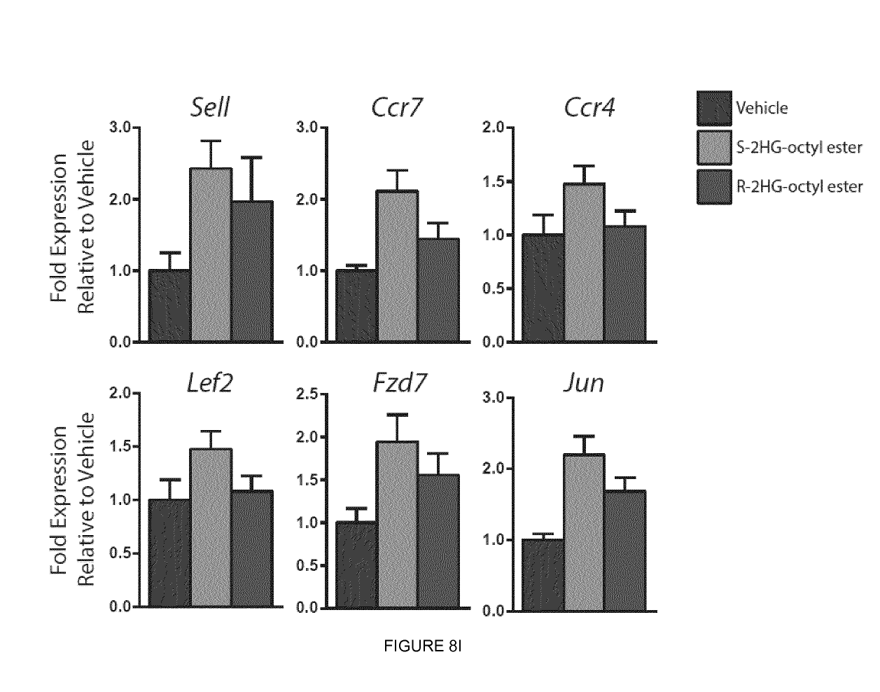

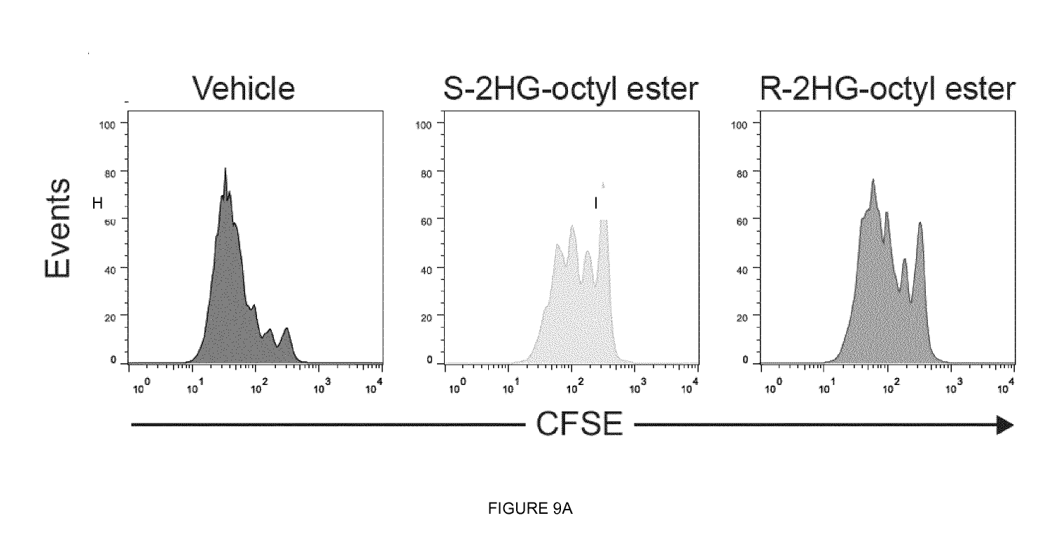

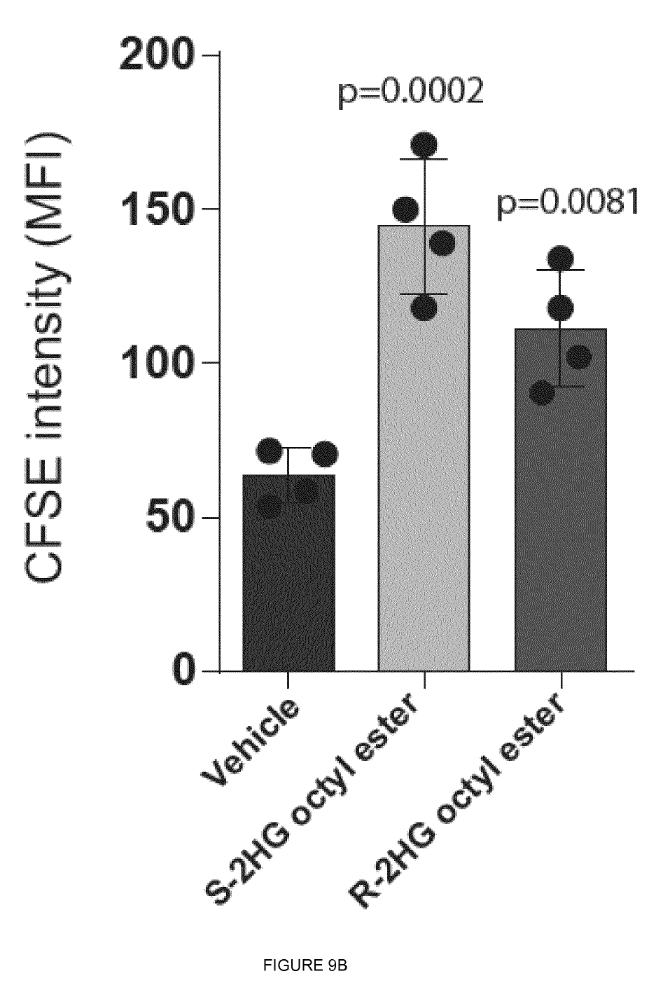

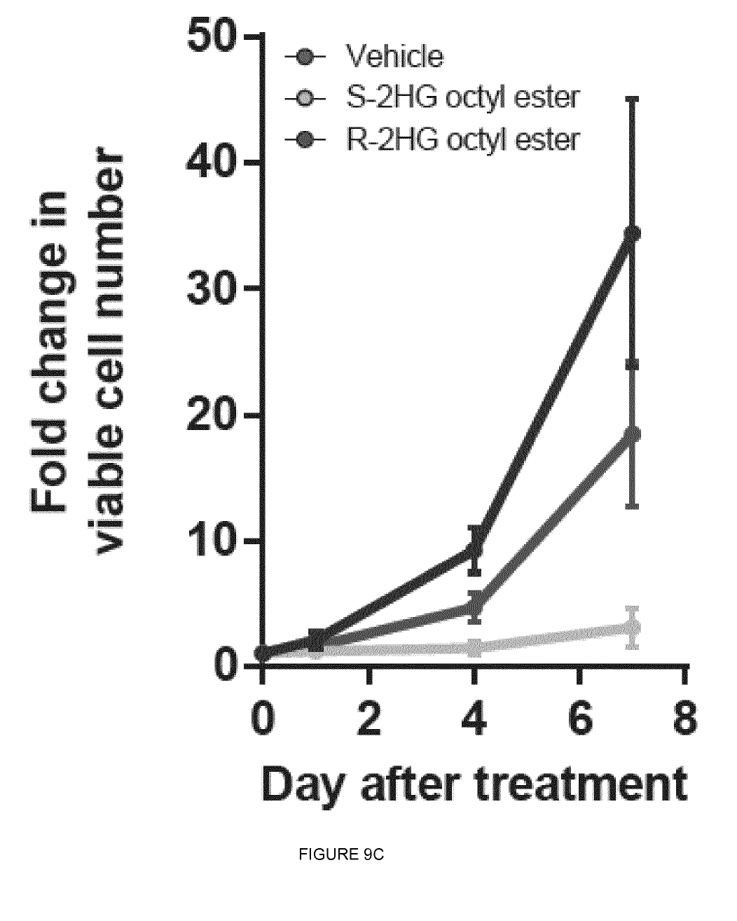

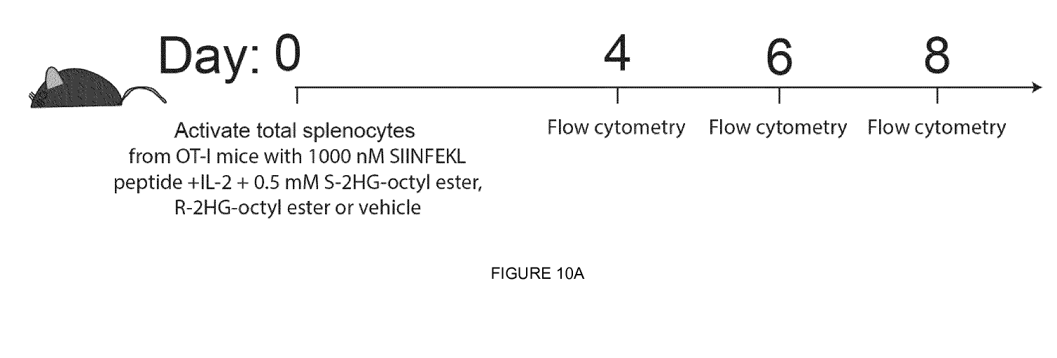

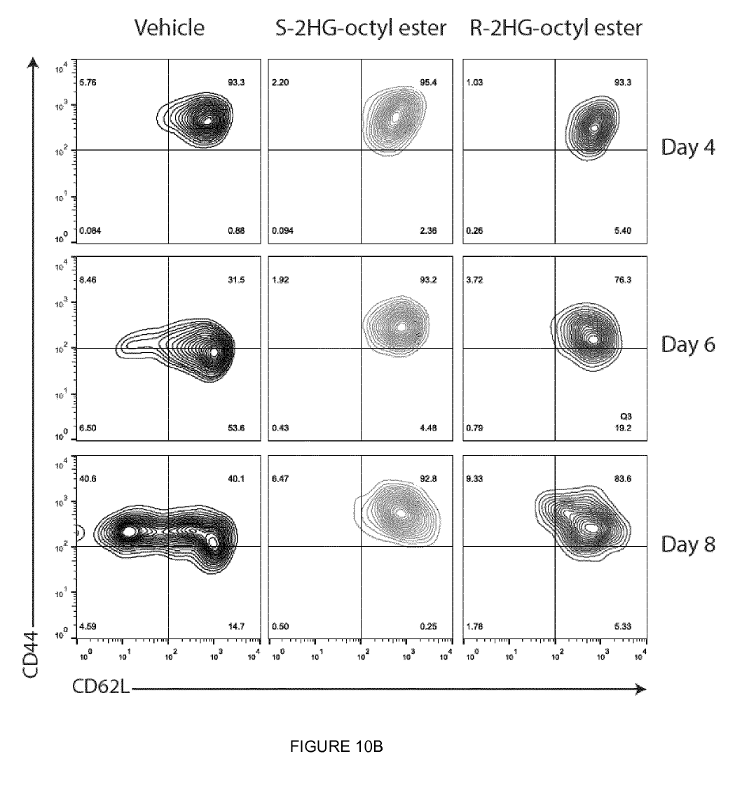

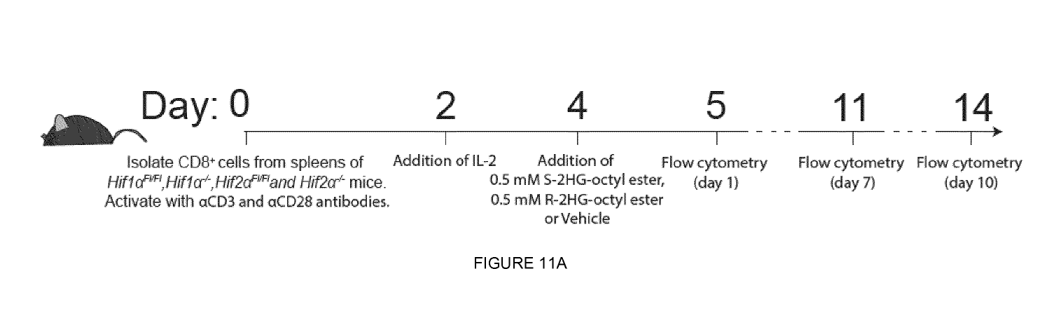

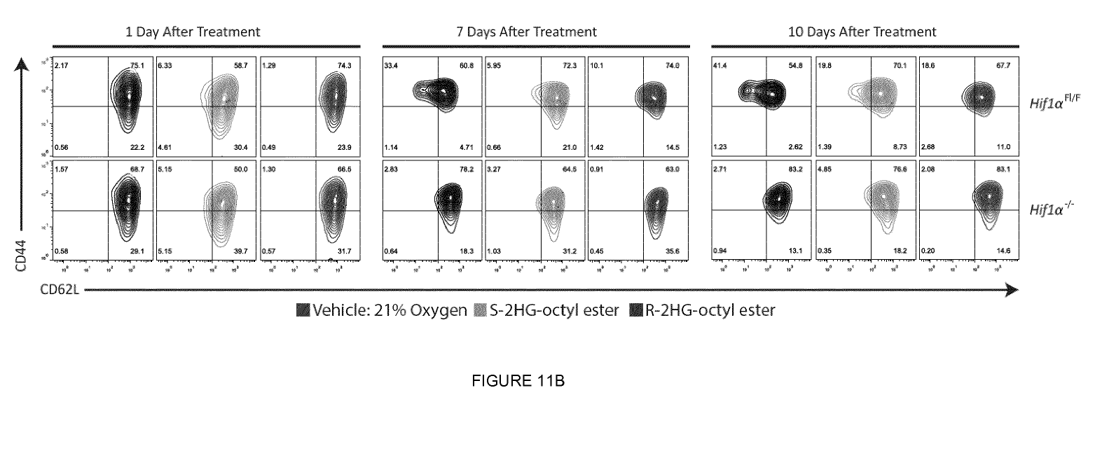

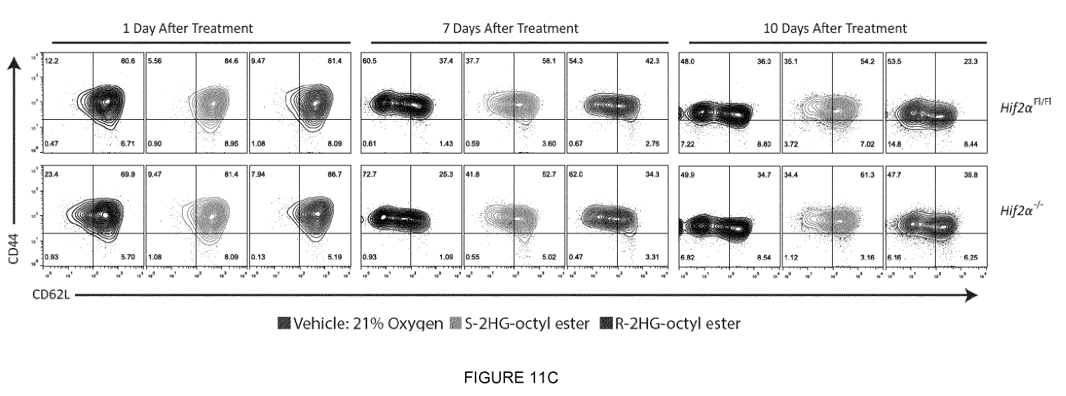

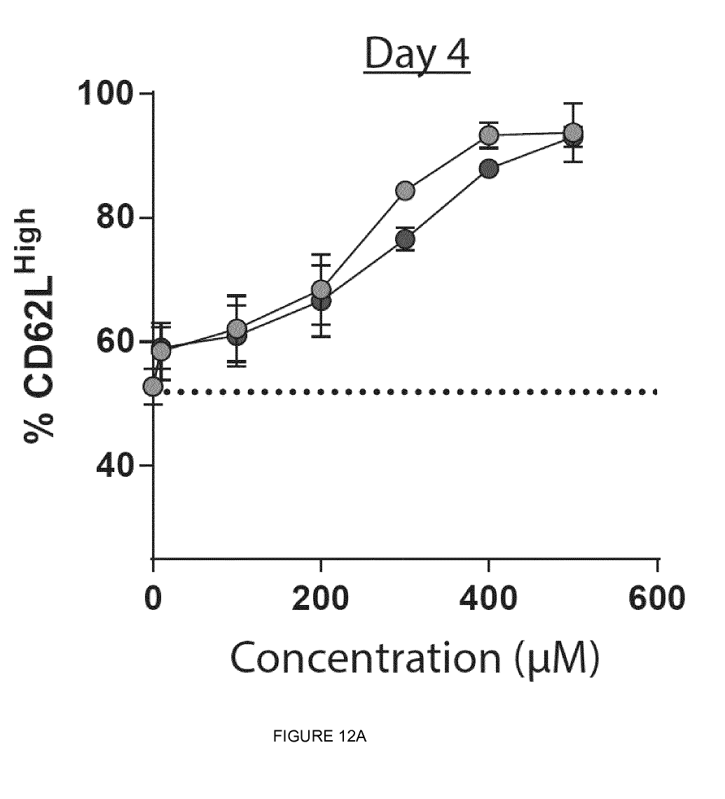

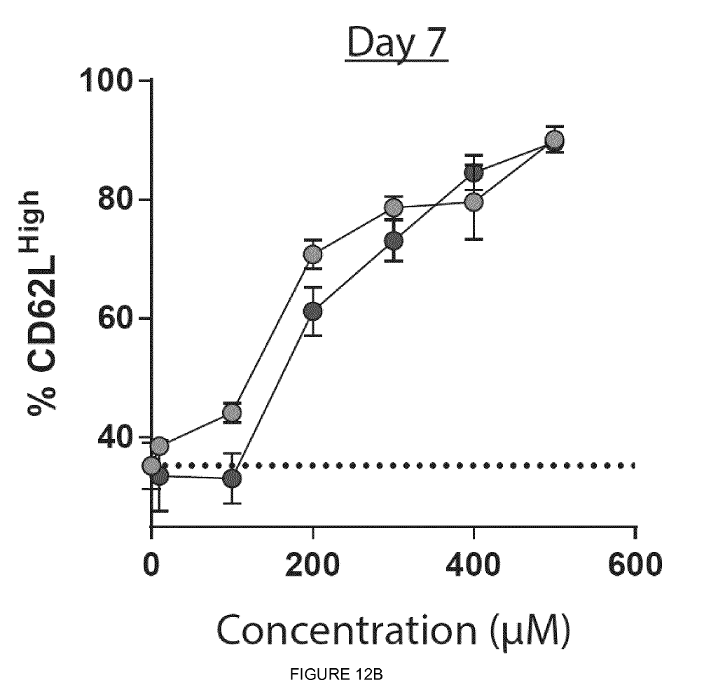

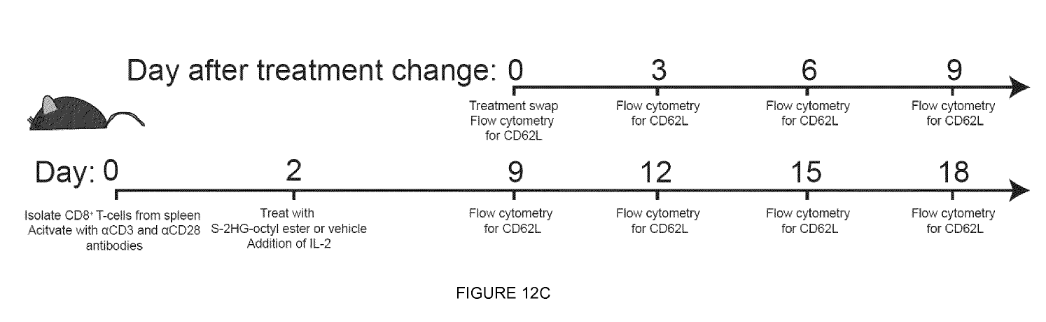

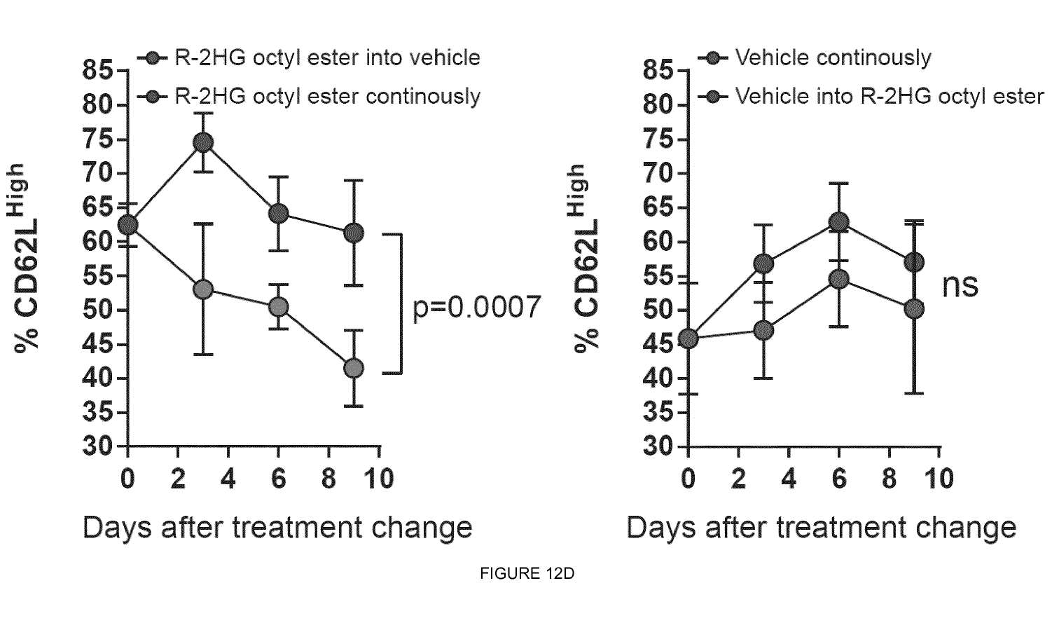

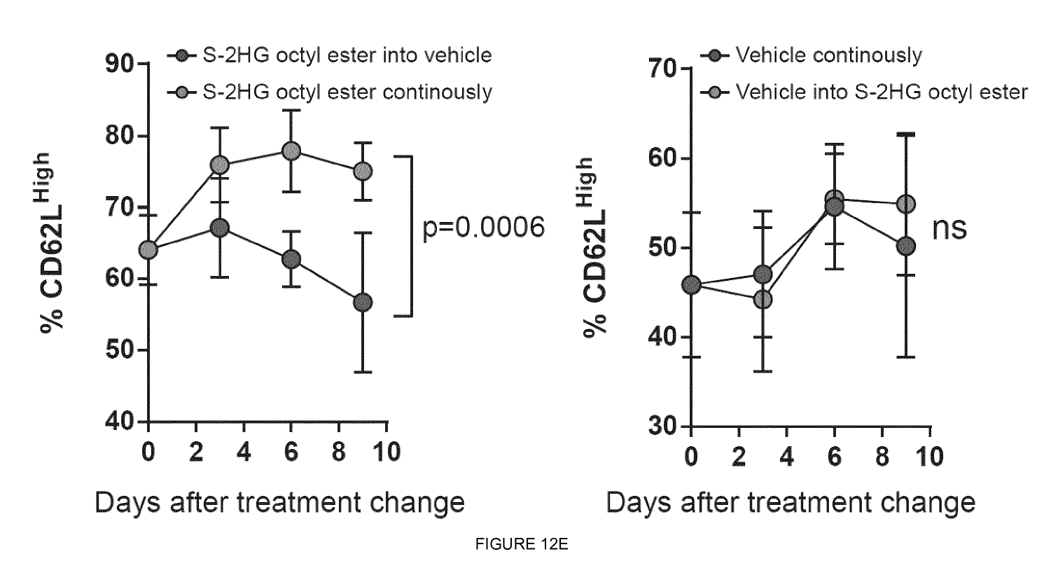

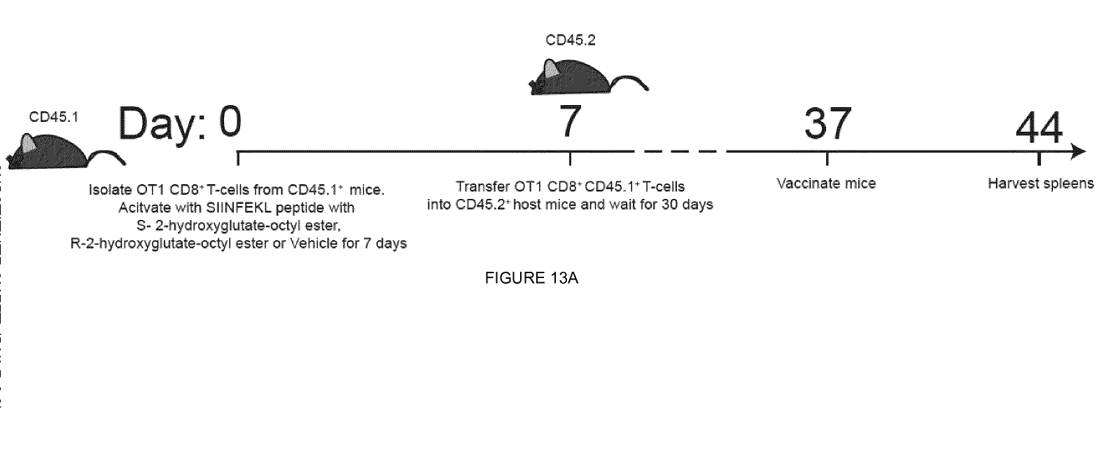

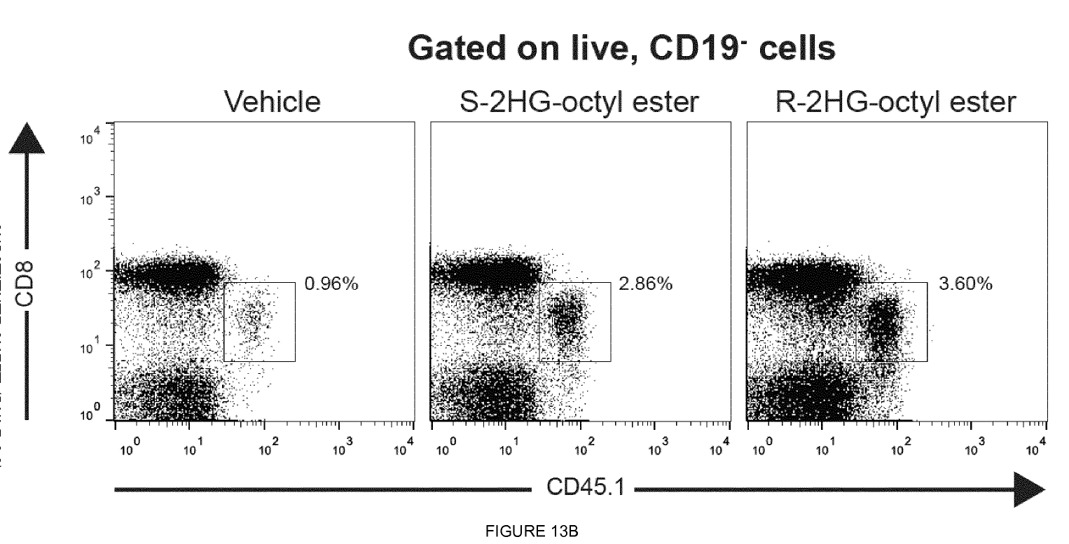

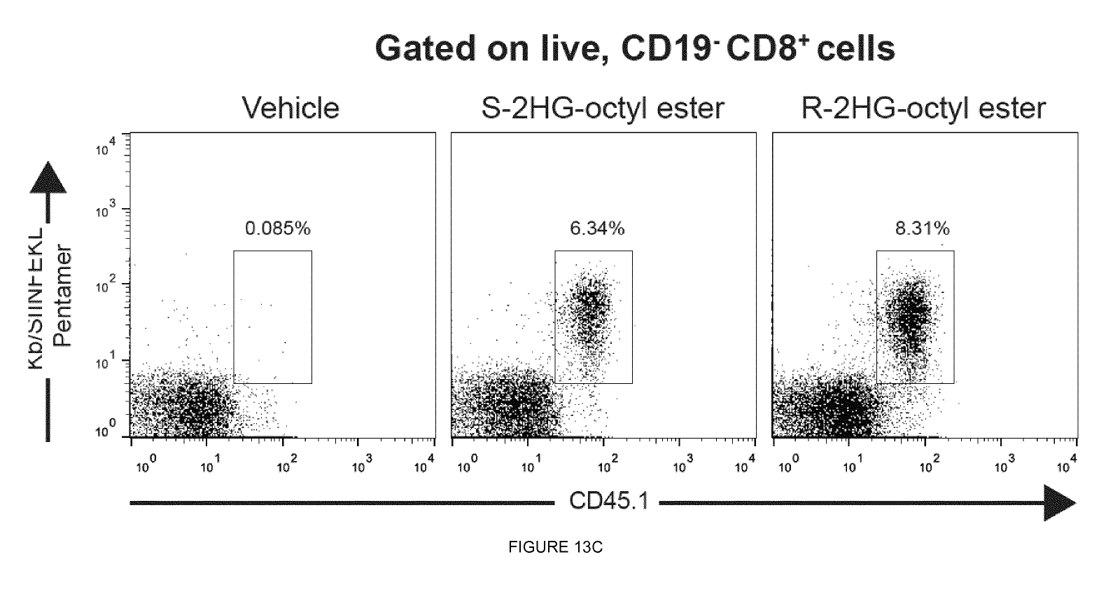

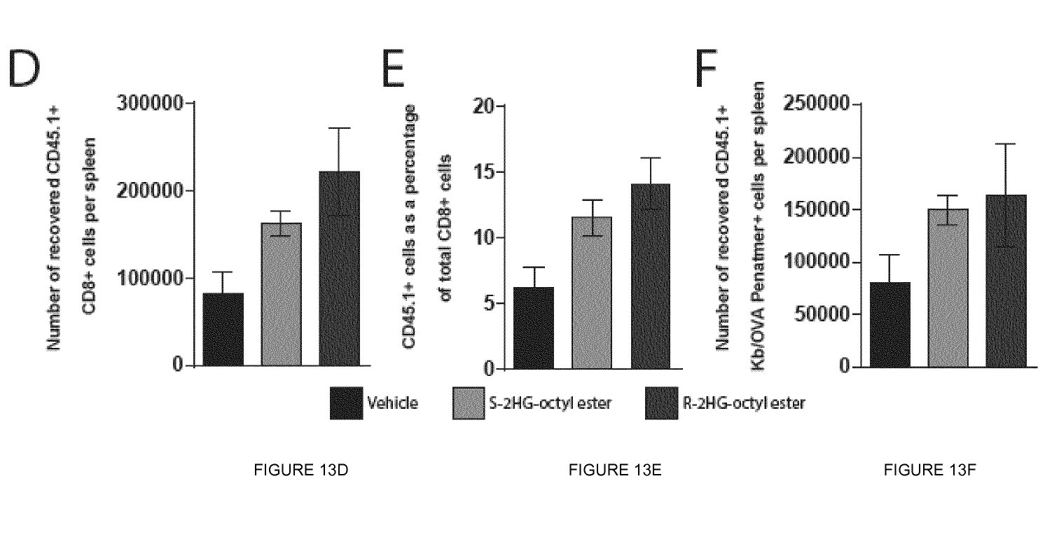

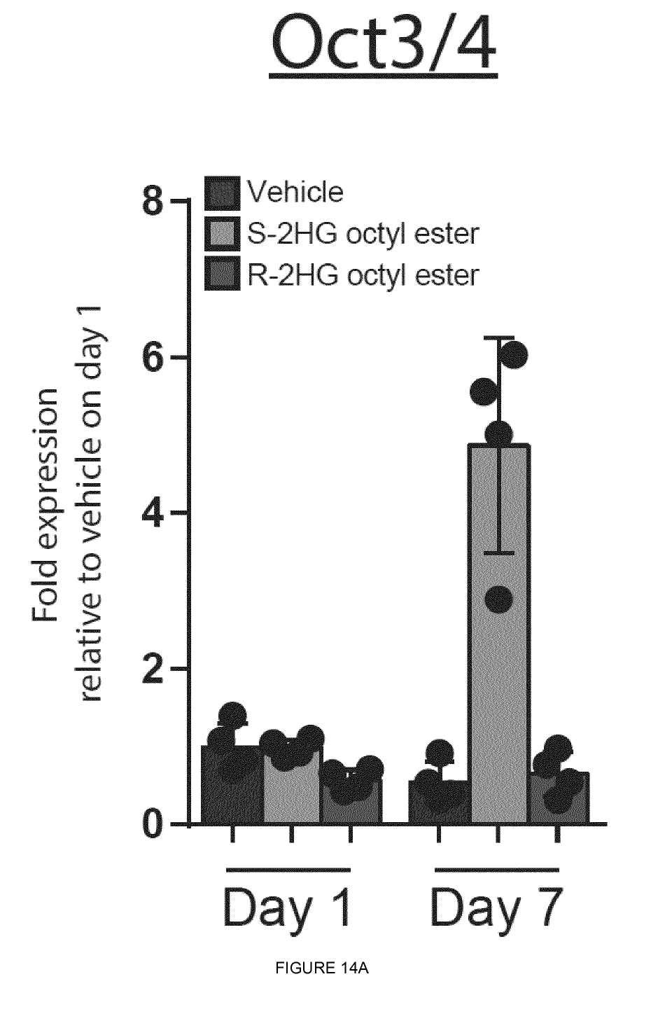

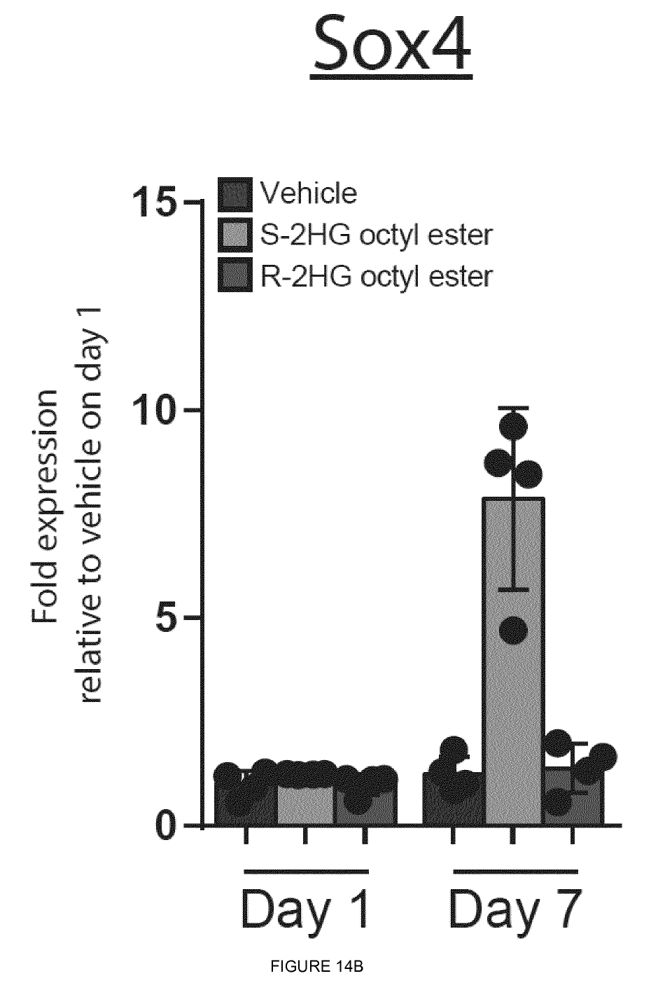

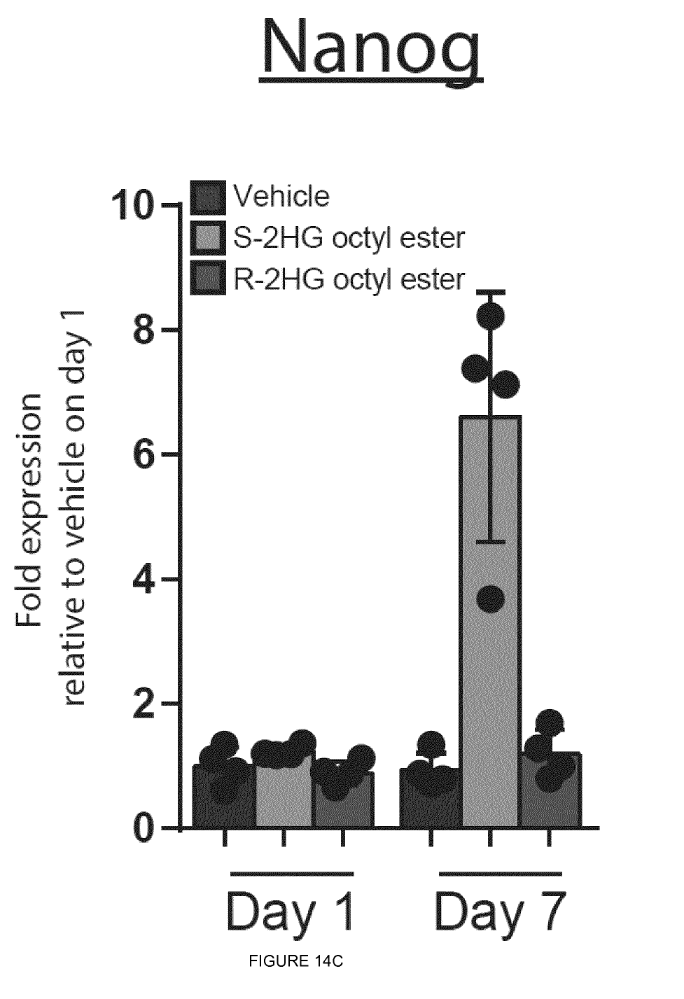

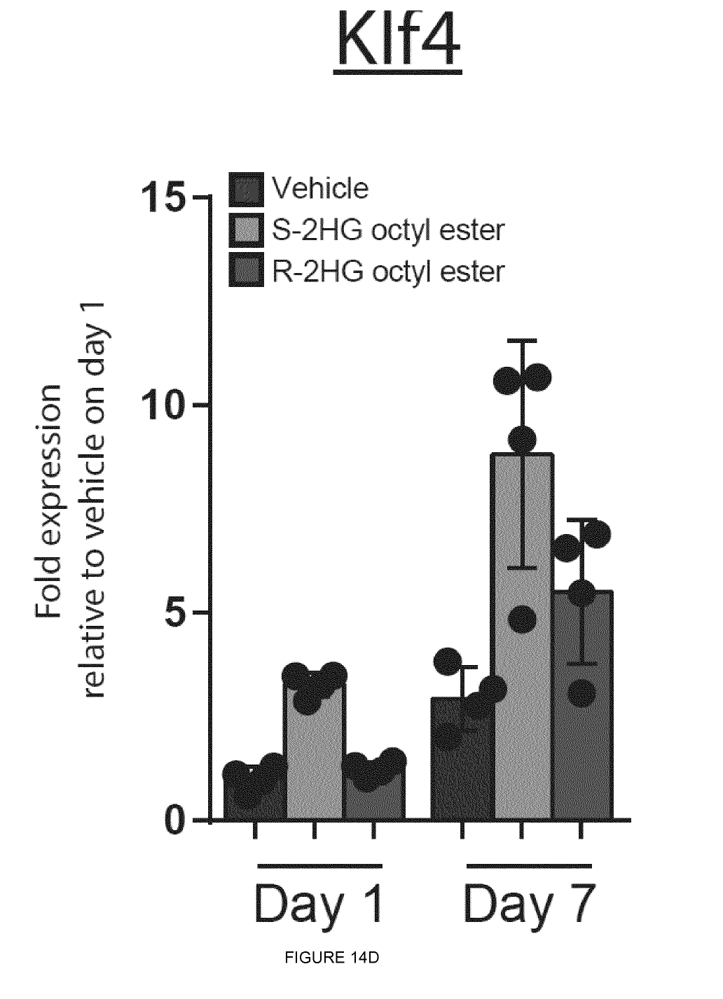

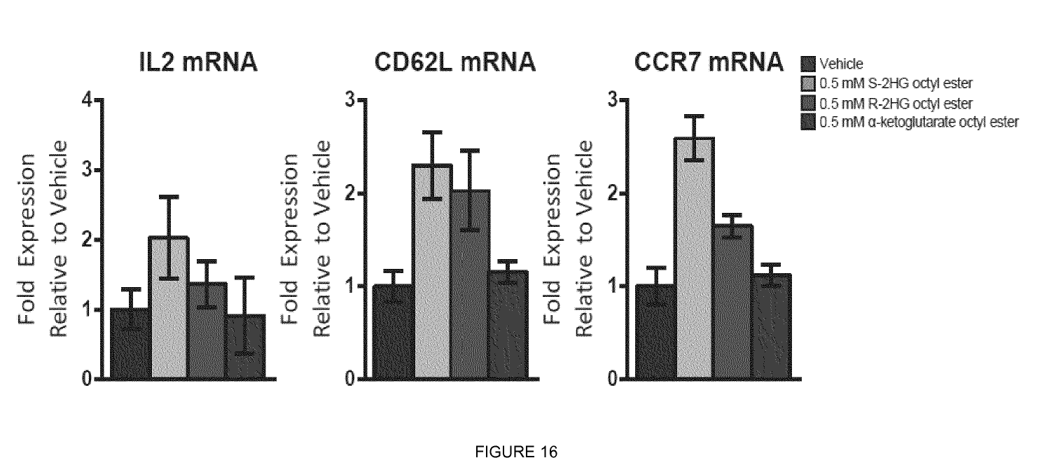

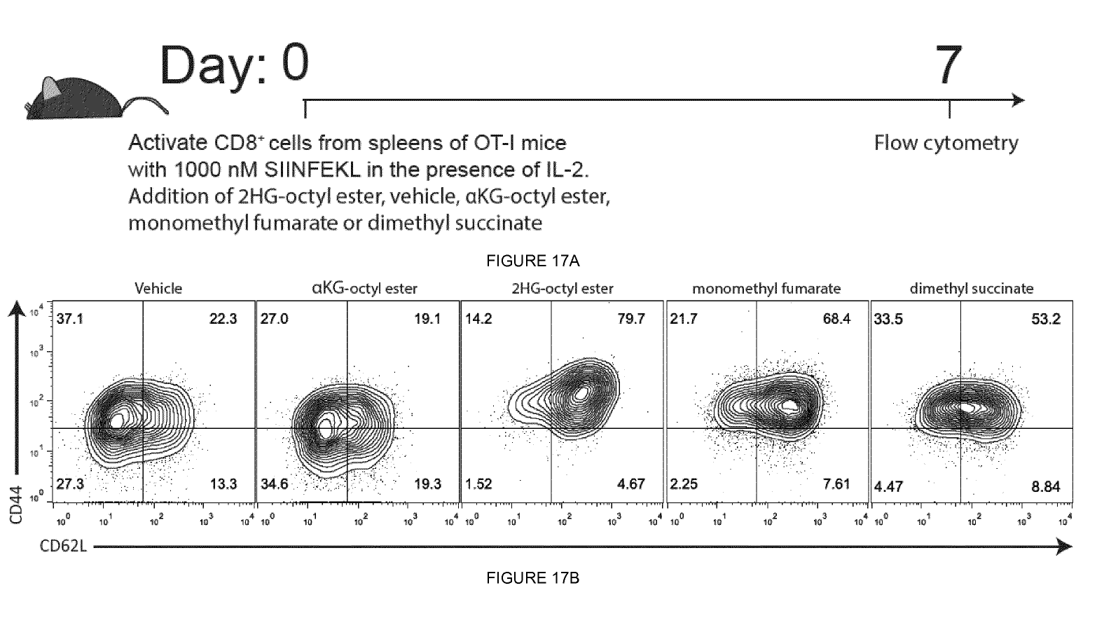

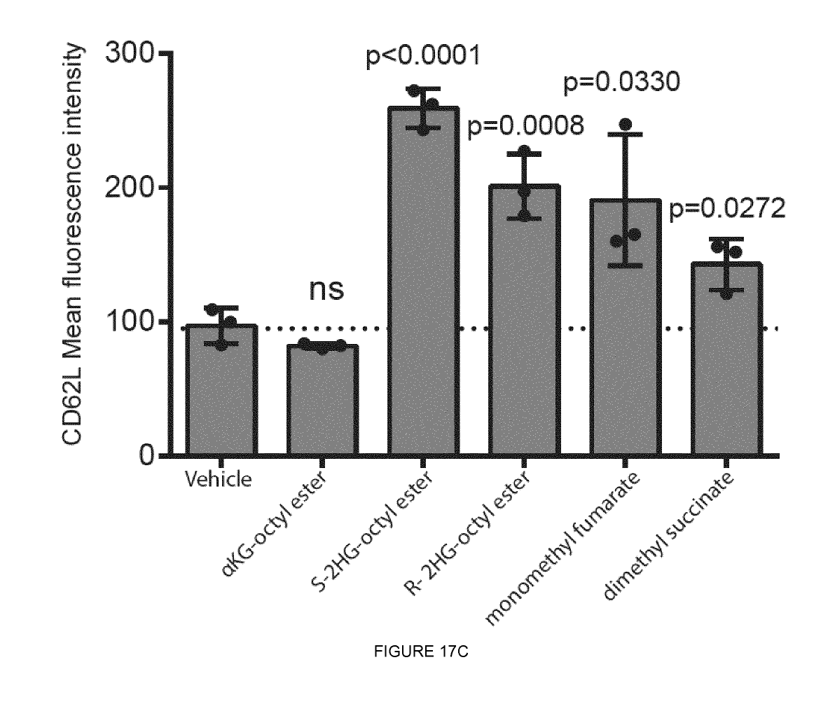

Further transcriptional profiling of genes involved in CD8⁺ T-lymphocyte differentiation and function indicates that both effector and memory programs are altered (FIG. 8G). In particular, acute treatment (24 h) with S-2HG-octyl ester or R-2HG-octyl ester represses the transcript levels of Eomes¹⁵, a key mediator of CD8⁺ T-lymphocyte differentiation²⁶, but then leads to higher expression levels after 7 days of continuous treatment (FIG. 8F), as well as decreasing proliferation following activation (FIG. 9A-C). The restriction in proliferation is less pronounced with R-2HG-octyl ester. Taken together, these data suggest that despite promoting HIF-1α activity, S-2HG and R-2HG do not encourage the formation of effector CD8⁺ T-lymphocytes, but rather those with memory-like features²⁷. Indeed, expression of memory associated transcripts is increased (FIG. 8I). We thus examined the levels of CD62L and CD44, two surface markers used to distinguish the differentiation status of CD8⁺ T-lymphocytes²⁸. S-2HG-octyl ester and R-2HG-octyl ester treatment promotes the formation of a CD62L^{high} CD44^{high} population in both antigen specific (FIGS. 10A and 10B) and polyclonal CD8⁺ T-lymphocyte settings (FIG. 11A-C). Furthermore, this effect depends on the level of antigenic stimulation (FIG. 8H), the dose of S-2HG-octyl ester or R-2HG-octyl ester (FIGS. 12A and 12B) and is also reversible upon withdrawal of treatment (FIGS. 12C, 12D and 12E). Interestingly, the effect does not occur when treating CD62L^{low} CD44^{high} effector cells (FIGS. 12D and12E), demonstrating that S-2HG-octyl ester or R-2HG-octyl ester treatment of newly activated naïve cells promotes the formation of memory-like subsets, rather than encouraging differentiation of already established effector populations. In addition, the effect was not observed when CD8⁺ T-lymphocytes were treated with α-ketoglutarate-octyl ester (FIGS. 16 and 17). Finally, adoptively transferred CD45.1 OT-1 CD8⁺ T-lymphocytes, pre-treated for 7 days with S-2HG-octyl ester or R-2HG-octyl ester (FIG. 13A), show enhanced in vivo recall in response to vaccination, 37 days after adoptive transfer (FIGS. 13B-F).

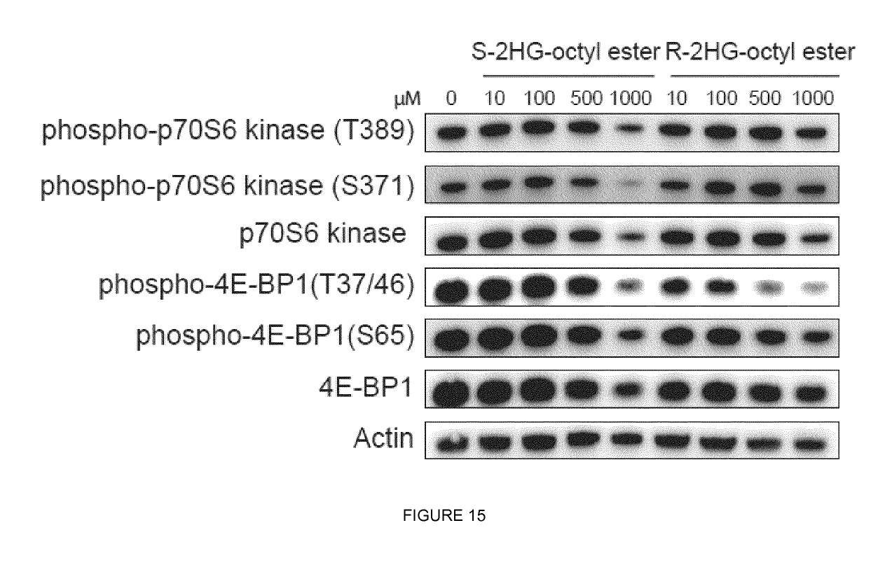

CD62L downregulation following activation in vitro does not occur when HIF-1α is absent²⁹; this loss of HIF-1α masks the effects of S-2HG-octyl ester and R-2HG-octyl ester treatment on CD62L (FIG. 11A, B). HIF-2a appears to play no role in CD62L downregulation, and thus S-2HG-octyl ester or R-2HG-octyl ester treatment inhibits CD62L downregulation in HIF-2a null cells to the same extent it does this in wild type controls (FIG. 11A, C). Thus it is possible that this effect is mediated not by the HIF pathway, but by modulation of other 2-oxoglutarate-dependent dioxygenases, e.g., the Jumonji C (JmjC) and Ten-eleven translocation (Tet) proteins, that epigenetically modify histones and DNA respectively^{4,30,31}. Reprogramming of metabolic pathways and modulation of mechanistic target of rapamycin (mTOR) activity are also known modifiers of CD8⁺ T-lymphocyte memory formation^{32,33,44, 45}; both of these are affected by 2HG^{10,34,46}. However, modulation of mTOR is not responsible for the induction of of memory-like CD8⁺ T-lymphocyte formation by S-2HG and R-2HG as described herein (FIG. 15), as the dose needed to inhibit mTOR far exceeds the dose necessary for memory formation.

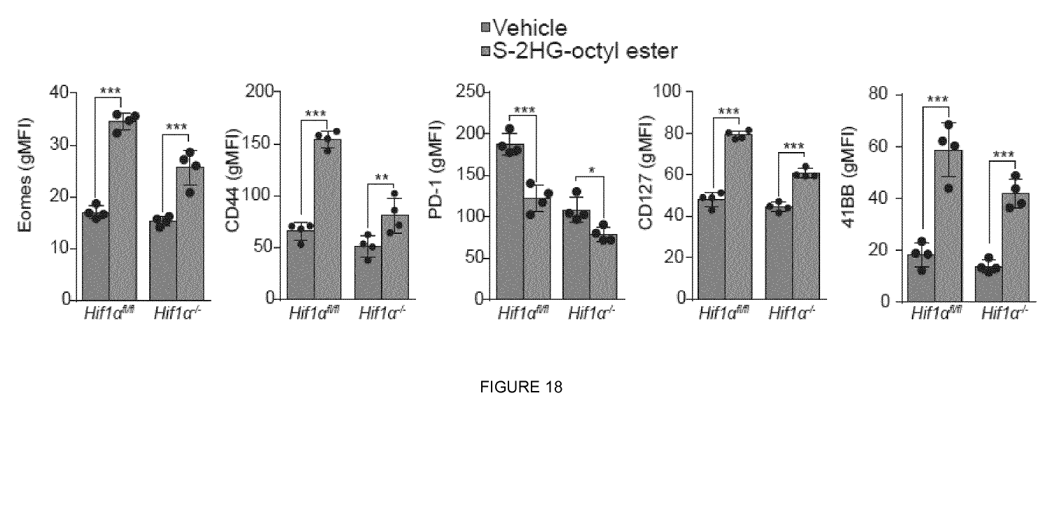

S-2HG treatment of cells induces the expression of pluripotency associated genes (Oct3/4, Sox2, Nanog, Klf4) (Fg.14). Furthermore, S-2HG treated CD8⁺ T-cells express more CD127 (FIG. 3c), CD44, 41BB and Eomes, in a HIF-1α-independent manner (FIG. 18). Interestingly, S-2HG treated cells also express less PD-1 (FIG. 18).

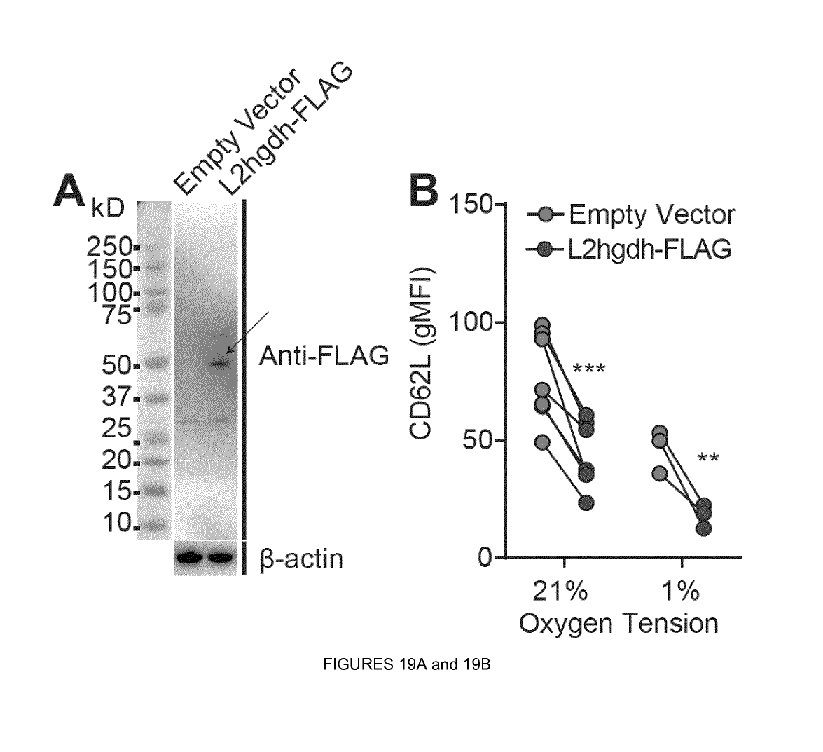

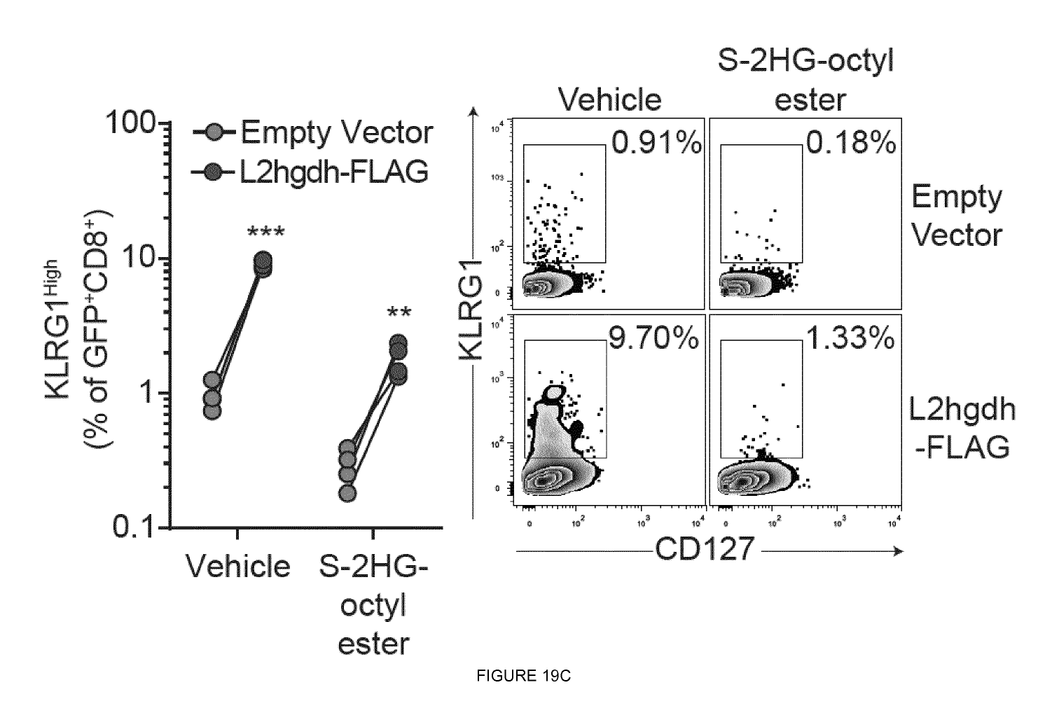

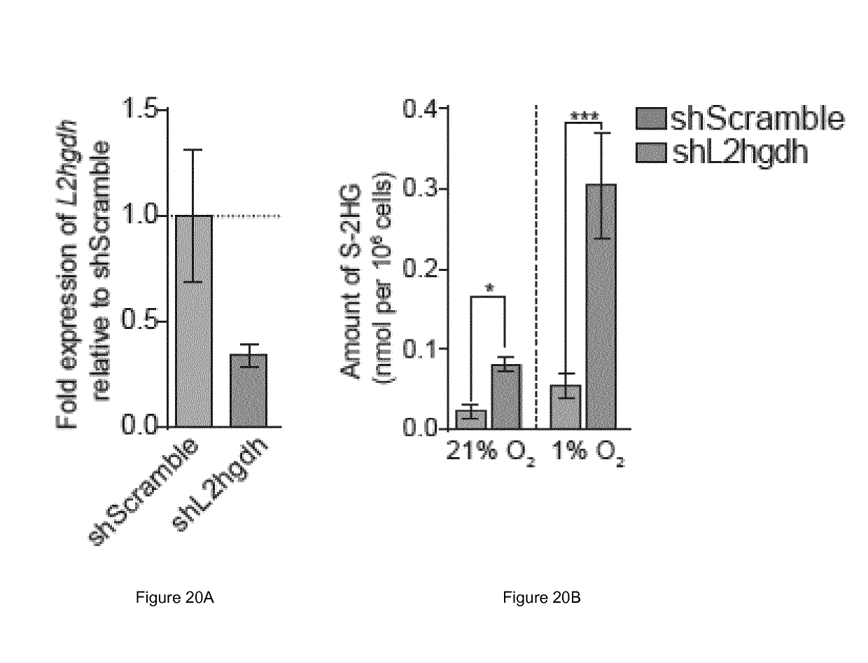

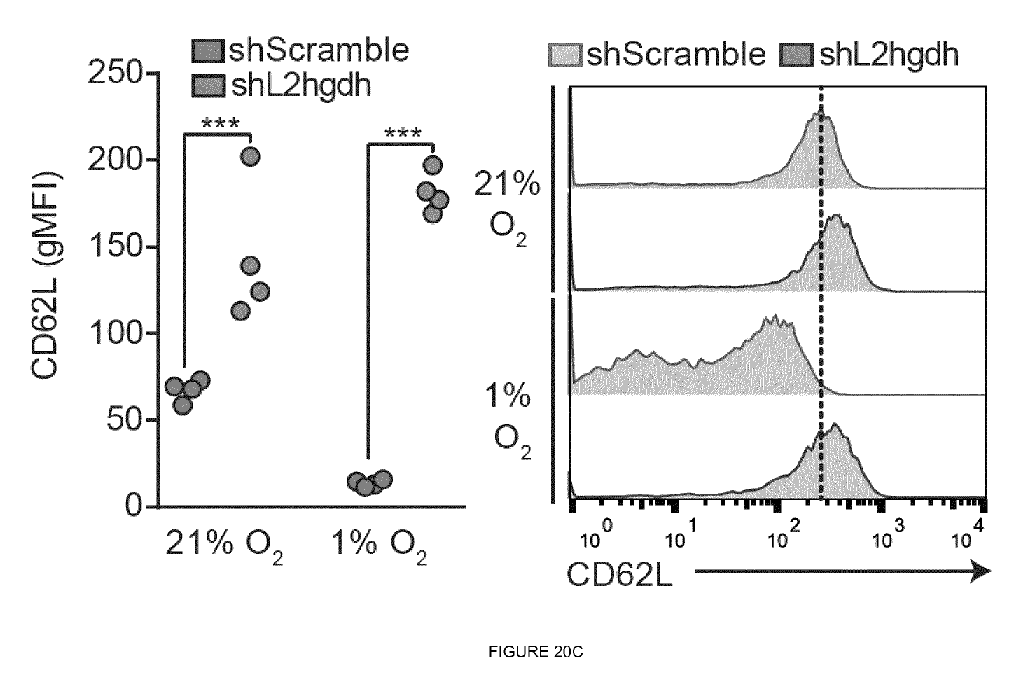

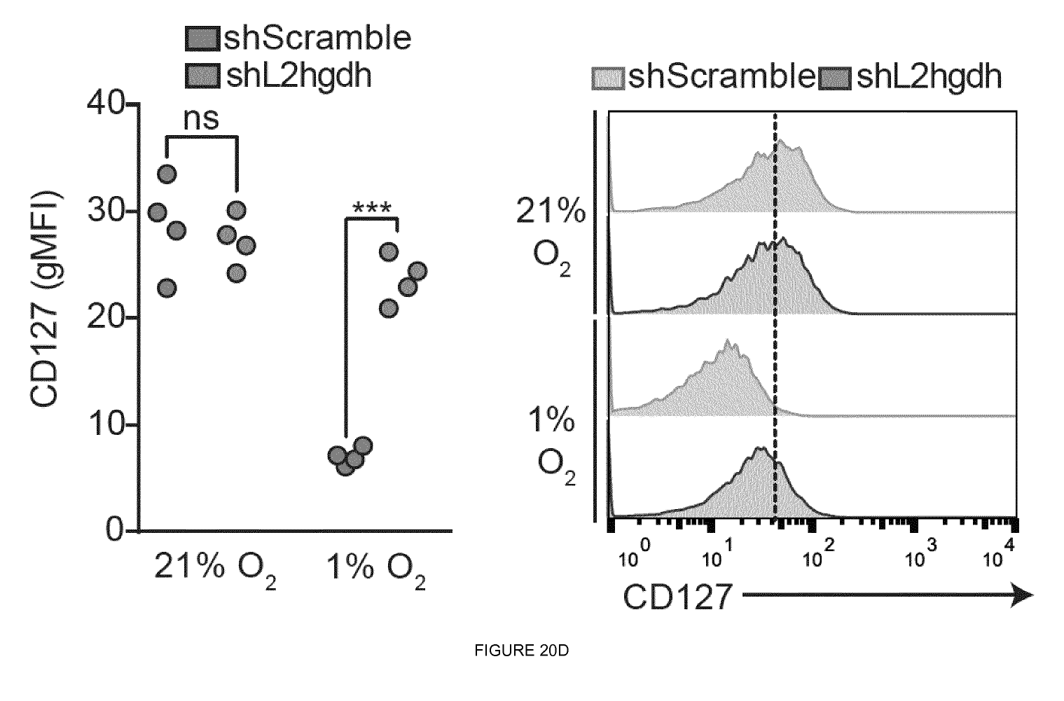

To determine the role of endogenously produced S-2HG in the absence of treatment with cell permeable S-2HG, overexpression of L-2-hydroxyglutarate dehydrogenase (L2hgdh) (FIG. 19A), a dehydrogenase that selectively oxidizes S-2HG to 2-oxoglutarate, was performed. Overexpression of L2hgdh promoted the downregulation of CD62L following activation in both 21% and 1% oxygen (FIG. 19B) indicating that endogenously produced S-2HG regulates CD62L expression. Furthermore, L2hgdh overexpression led to an increase in the proportion of KLRG1^{High} cells, which are decreased in the presence of exogenous S-2HG (FIG. 19C). Conversely, successful shRNA-mediated knockdown of L2hgdh (FIG. 20A) increased endogenous S-2HG levels (FIG. 20B), especially in 1% oxygen, and promoted the maintenance of CD62L (FIG. 20C). In fact, suppression of L2hgdh blocked the loss of CD62L in response to low oxygen exposure (FIG. 20C). The same effect is seen with CD127 abundance in low oxygen conditions (FIG. 20D). These data demonstrated that L2hgdh activity regulates the expression of key phenotypic surface makers on CD8⁺ T-lymphocytes, by controlling endogenous S-2HG levels. These phenotypic effects are found in memory CD8⁺ T-lymphocytes, providing indication that S-2HG treatment of CD8⁺ T-lymphocytes ex vivo may enhance long term persistence and survival in the context of adoptive cell transfer⁴⁷.

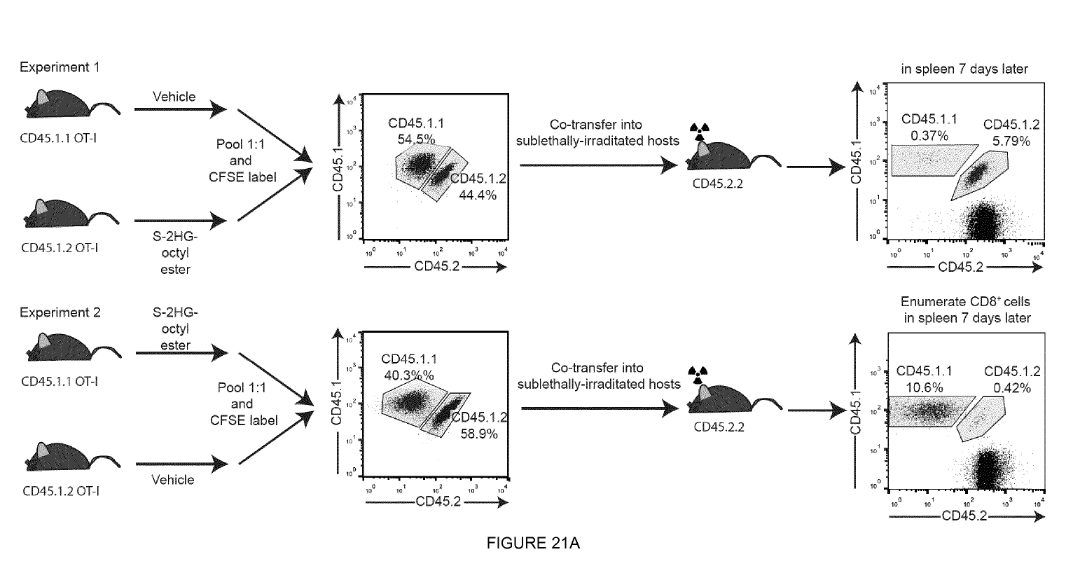

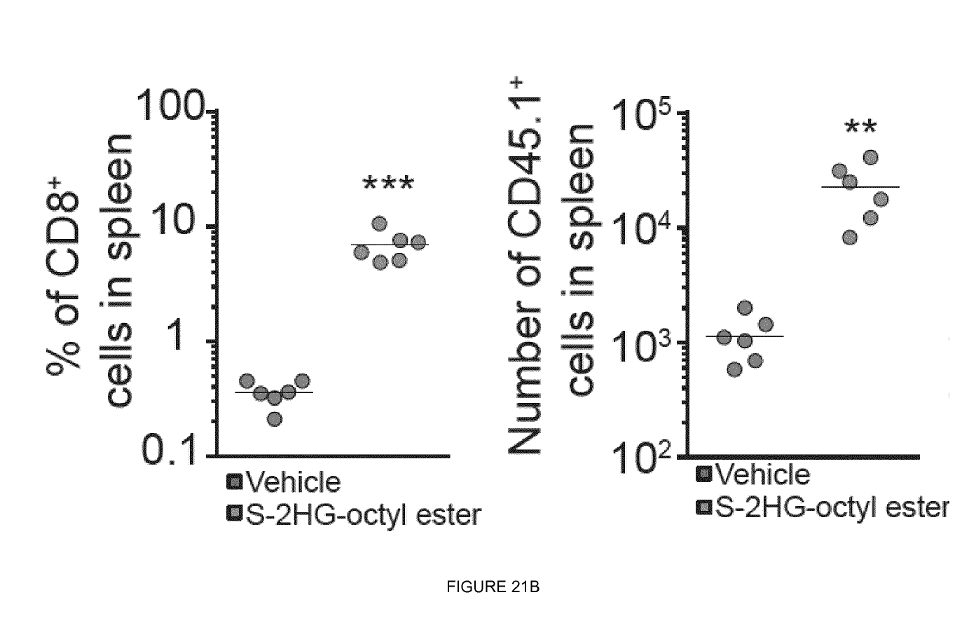

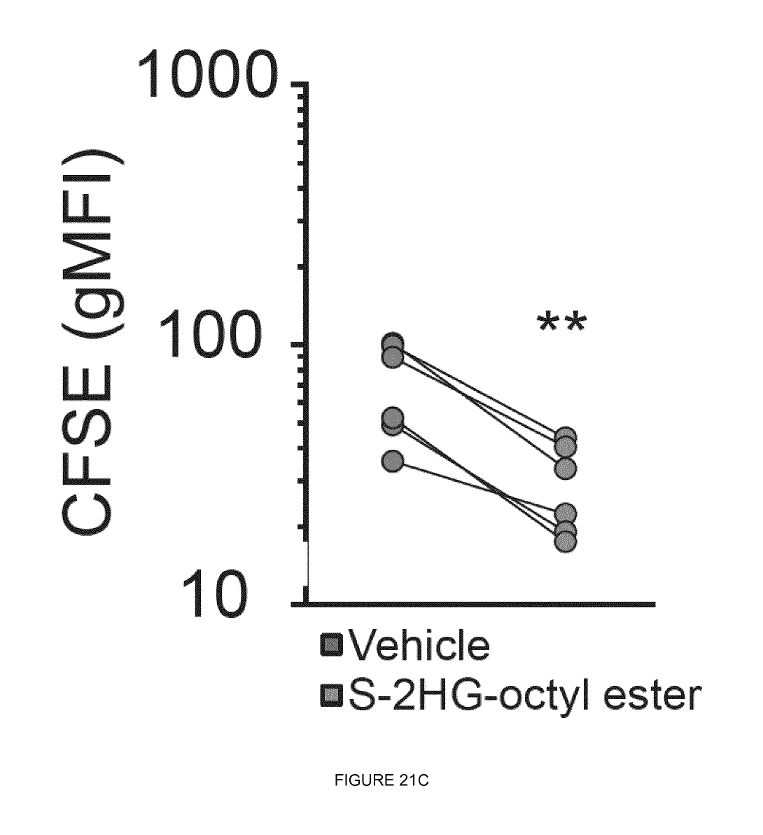

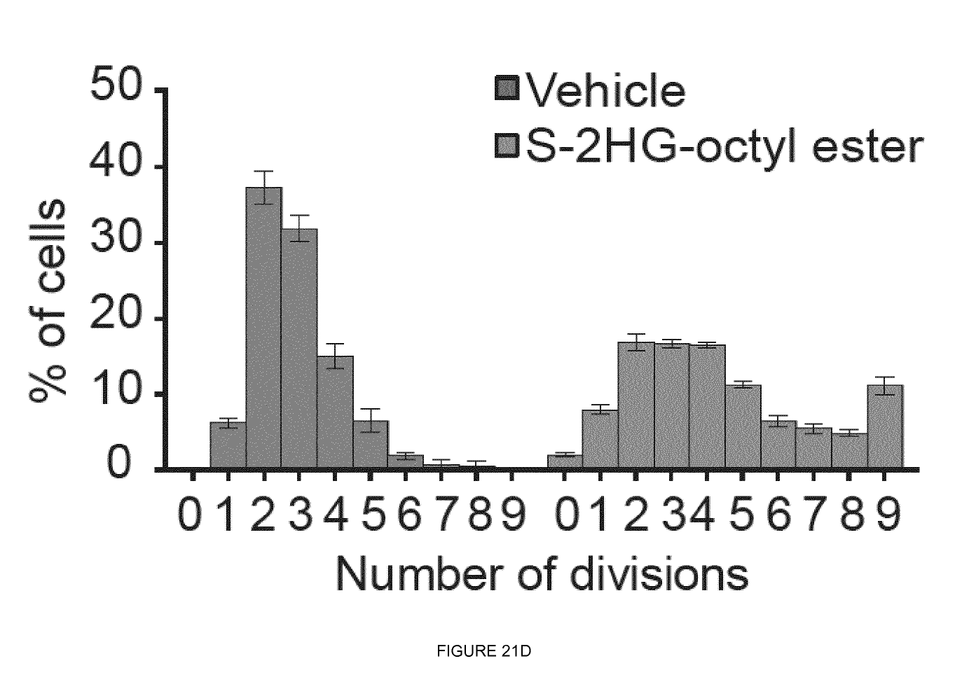

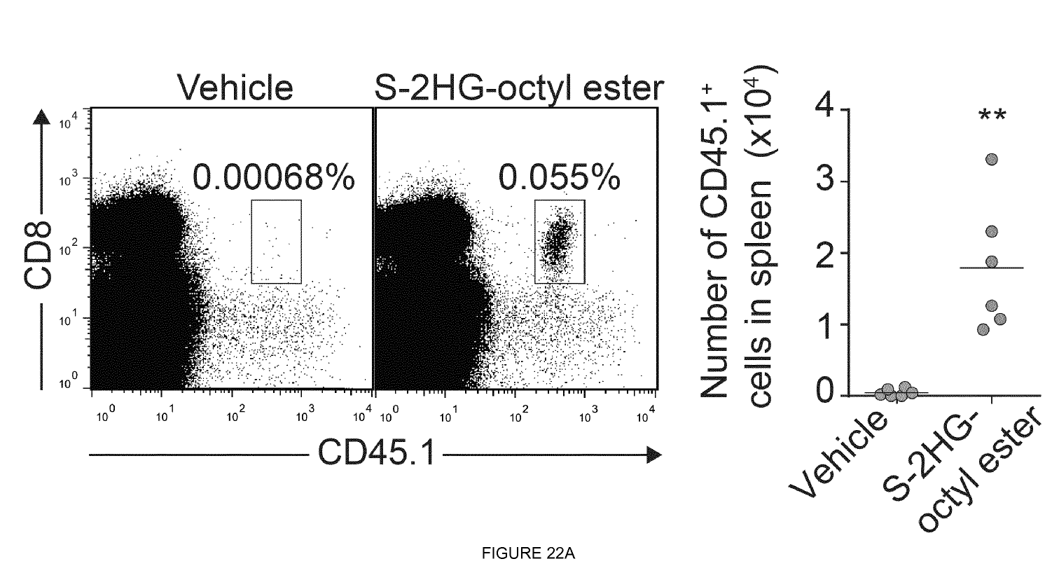

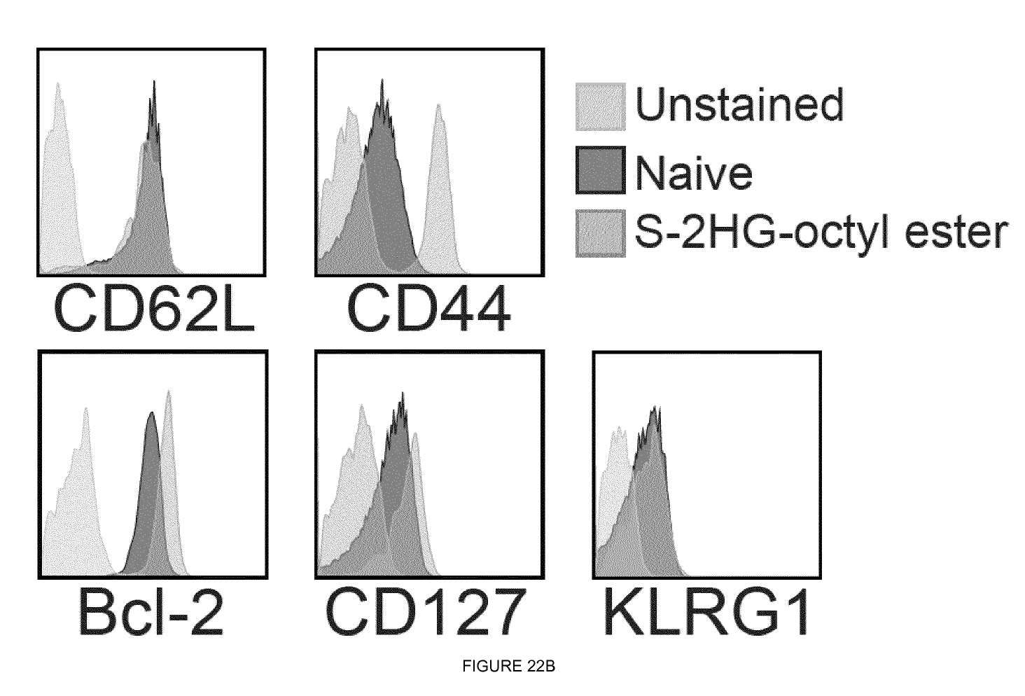

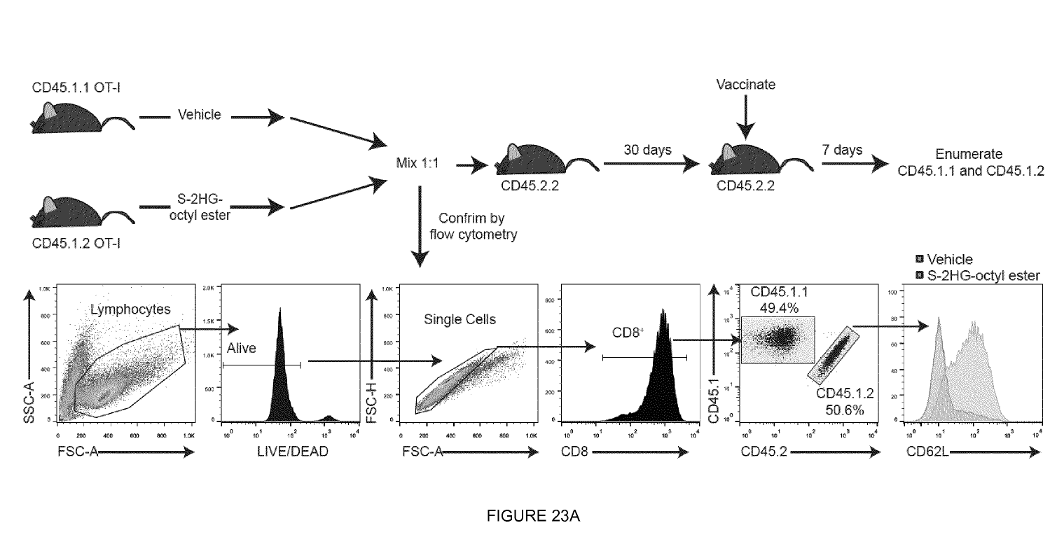

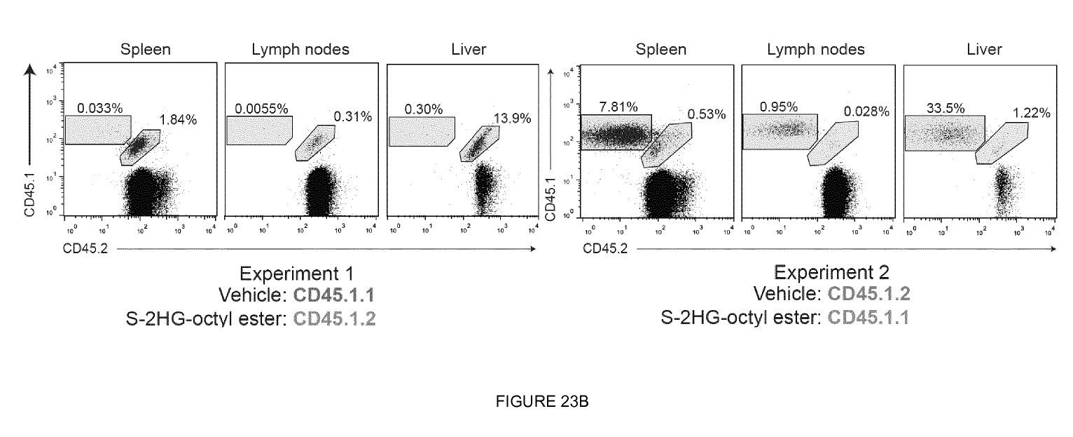

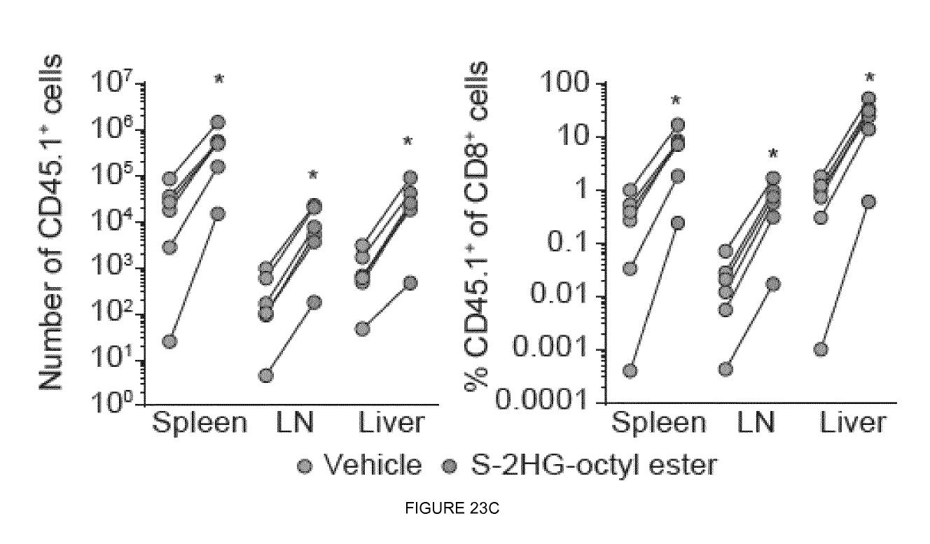

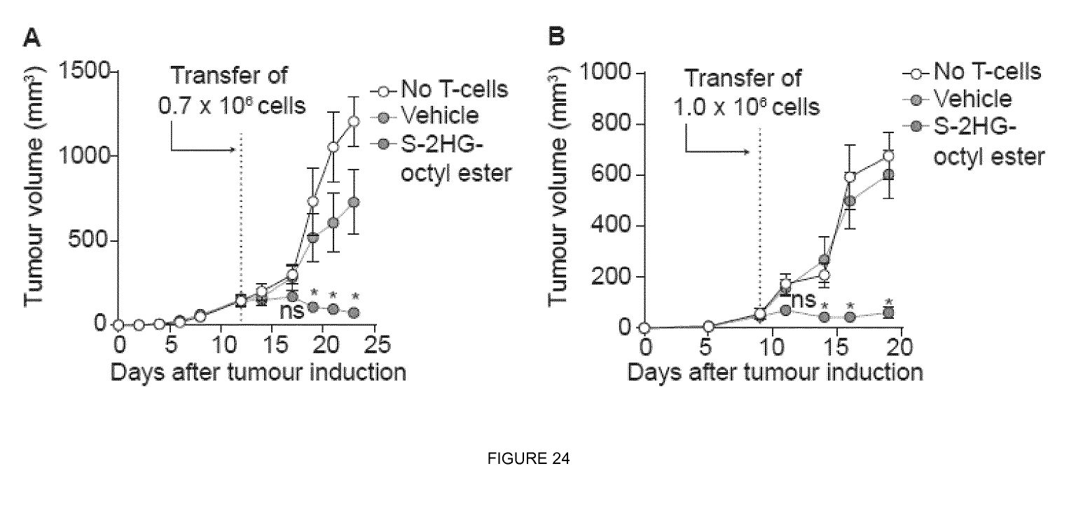

We thus co-transferred CFSE-labelled vehicle and S-2HG treated CD45.1.1 or CD45.1.2 OT-I CD8⁺ T-lymphocytes into lymphodepleted hosts (FIG. 21A) to assess their capacity for homeostatic proliferation, which is a hallmark of memory cells^{48, 49}. Due to imperfect mixing, S-2HG-treated cells in both experiments were at a numerical disadvantage when pooled with vehicle-treated cells just before co-transfer (FIG. 21A). Despite this, S-2HG-treated cells displayed greater homeostatic proliferation (FIG. 21B-C), with more cells dividing >5 times (FIG. 21D). Given this, we then assessed the capacity of S-2HG treated cells to persist for long periods in vivo. Adoptively transferred CD45.1 OT-I CD8⁺ T-lymphocytes, pre-treated with S-2HG, showed dramatically enhanced persistence 30 days after transfer (FIG. 22A). Furthermore, they expressed elevated CD44, CD127 and Bcl-2 levels relative to naïve cells (FIG. 22B), markers that are expressed by memory cells^{50, 51}. In response to a vaccination with SIINFEKL-loaded dendritic cells, S-2HG-treated OT-I CD8⁺ T-lymphocytes mounted a superior recall response (FIG. 23A-C). Consistent with this, OT-I CD8⁺ T-lymphocytes, pre-treated with S-2HG are more proficient at controlling tumour growth in vivo in both lymphodepleted (FIG. 24A) and lymphoreplete (FIG. 24B) tumour-bearing mice. Together, these data demonstrated that S-2HG treatment ex vivo maintained cells in a state with increased proliferative and survival capacity, when transferred in vivo, that is otherwise decreased by effector differentiation.

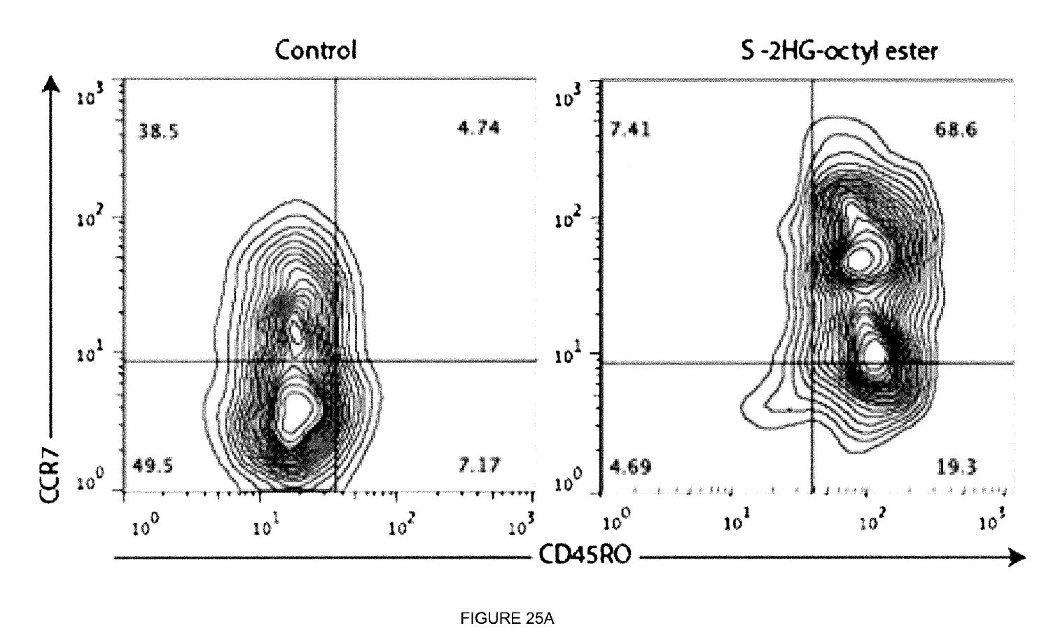

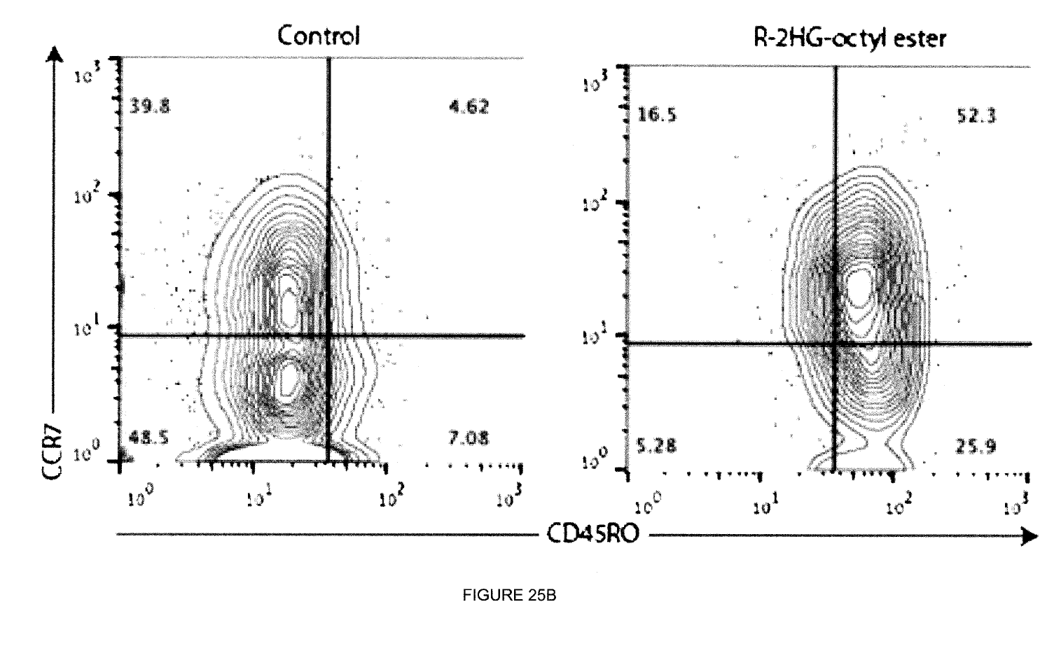

To determine the ability of S-2HG-octyl ester and R-2HG-octyl ester to alter human T cell differentiation, we isolated and activated human CD8⁺ T cells from healthy donors in vitro. After expansion in the presence of vehicle control, 600 μM S-2HG-octyl ester (FIG. 25A) or 800 μM R-2HG-octyl ester (FIG. 25B), we checked the expression of human T cell memory markers CCR7 and CD45RO by flow cytometry. Both S-2HG-octyl ester and R-2HG-octyl ester induced the expression of CCR7 and CD45RO when compared to respective vehicle controls.

Our data show that physiological production of S-2HG and R-2HG induces formation of memory-like CD8⁺ T-lymphocyte populations, allowing for the modulation of immunity in a context-dependant manner. Clearly, pharmacologic administration of these metabolites and other memory induction compounds has a striking potential for therapeutic manipulation of T cell responsiveness, and provides a new strategy to enhance activity in adoptive T cell therapies.

### REFERENCES

1 Dang, L. et al. Nature 462, 739-744, doi:10.1038/nature08617 (2009).
2 Choi, C. et al. Nat Med 18, 624-629, doi:10.1038/nm.2682 (2012).
3 Koivunen, P. et al. Nature 483, 484-488, doi:10.1038/nature10898 (2012).
4 Xu, W. et al Cancer Cell 19, 17-30, doi:10.1016/j.ccr.2010.12.014 (2011).
5 Lu, C. et al. Nature 483, 474-478, doi:10.1038/nature10860 (2012).
6 Figueroa, M. E. et al. Cancer Cell 18, 553-567, doi:10.1016/j.ccr.2010.11.015 (2010).
7 Losman, J. A. et al. Science 339, 1621-1625, doi:10.1126/science.1231677 (2013).
8 Gibson, K. M. et al. Pediatr Res 34, 277-280, doi:10.1203/00006450-199309000-00007 (1993).
9 Intlekofer, A. M. et al. Cell metabolism 22, 304-311, doi:10.1016/j.cmet.2015.06.023 (2015).
10 Oldham, W. M. et al Cell metabolism 22, 291-303, doi:10.1016/j.cmet.2015.06.021 (2015).
11 Palazon, A. et al Immunity 41, 518-528, doi:10.1016/j.immuni.2014.09.008 (2014).
12 Wang, R. et al. Immunity 35, 871-882, doi:10.1016/j.immuni.2011.09.021 (2011).
13 Pearce, E. L. et al. Nature 460, 103-107, doi:10.1038/nature08097 (2009).
14 O'Sullivan, D. et al. Immunity 41, 75-88, doi:10.1016/j.immuni.2014.06.005 (2014).
15 Doedens, A. L. et al. Nat Immunol 14, 1173-1182, doi:10.1038/ni.2714 (2013).
16 Ghesquiere, B. et al Nature 511, 167-176, doi:10.1038/nature13312 (2014).
17 Losman, J. A. et al Genes Dev 27, 836-852, doi:10.1101/gad.217406.113 (2013).
18 Saha, S. K. et al. Nature 513, 110-114, doi:10.1038/nature13441 (2014).
19 Wise, D. R. et al. Proc Natl Acad Sci USA 108, 19611-19616, doi:10.1073/pnas.1117773108 (2011).
20 Mullen, A. R. et al Cell Rep 7, 1679-1690, doi:10.1016/j.celrep.2014.04.037 (2014).
21 Shim, E. H. et al. Cancer discovery 4, 1290-1298, doi:10.1158/2159-8290.CD-13-0696 (2014).
22 Patel, K. P. et al. J Mol Diagn 13, 678-686, doi:10.1016/j.jmoldx.2011.06.004 (2011).
23 Zhang, D. J. eta IJournal of immunology 174, 6725-6731 (2005).
24 Metallo, C. M. et al. Nature 481, 380-384, doi:10.1038/nature10602 (2012).
25 Fendt, S. M. et al. Nat Commun 4, 2236, doi:10.1038/ncomms3236 (2013).
26 Pearce, E. L. et al. Science 302, 1041-1043, doi:10.1126/science.1090148 (2003).
27 Gattinoni, L. et al. Nat Med 15, 808-813, doi:10.1038/nm.1982 (2009).
28 Gattinoni, L. et al. The Journal of clinical investigation 115, 1616-1626, doi:10.1172/JC124480 (2005).
29 Finlay, D. K. et al. J Exp Med 209, 2441-2453, doi:10.1084/jem.20112607 (2012).
30 Ko, M. et al. Nature 468, 839-843, doi:10.1038/nature09586 (2010).
31 Chowdhury, R. et al. EMBO Rep 12, 463-469, doi:10.1038/embor.2011.43 (2011).
32 Sukumar, M. et al. The Journal of clinical investigation 123, 4479-4488, doi:10.1172/JC169589 (2013).
33 Araki, K. et al. Nature 460, 108-112, doi:10.1038/nature08155 (2009).
34 Fu, X. et al. Cell metabolism, doi:10.1016/j.cmet.2015.06.009 (2015).
35 Maclver, N. et al. Annual review of immunology 31, 259-283, doi:10.1146/annurev immunol-032712-095956 (2013).
36 Haase, V. H.et al PNAS USA 98, 1583-1588, doi:10.1073/pnas.98.4.1583 (2001).
37 Gruber, M. et al. PNAS USA 104, 2301-2306, doi:10.1073/pnas.0608382104 (2007).
38 Hogquist, K. A. et al. Cell 76, 17-27 (1994).
39 Jung, S. et al. Molecular and cellular biology 20, 4106-4114 (2000).
40. Gattinoni, L. et al. Nat. Med. 17, 1290-1297 (2011)
41. Restifo, N. P. et al Nat. Rev. Immunol. 12, 269-281 (2012)
42. WO 2014039044
43. WO 2010151517
44. U.S. Pat. No. 9,057,054
45. U.S. Pat. No. 8,840,899
46. Fu et al Cell Metab (2015) 22 (3) 508-151
Arsenio, J. et al. s. Nat Immunol 15, 365-372, doi:10.1038/ni.2842 (2014).
Goldrath, A. W. et al. J Exp Med 195, 1515-1522 (2002).
Murali-Krishna, K. et al. Science 286, 1377-1381 (1999).
Araki, K. et al. Nature 460, 108-112, doi:10.1038/nature08155 (2009).
51 Grayson, J. M. et al. Journal of immunology 164, 3950-3954 (2000).

1. A method of expanding a population of memory-like T-lymphocytes comprising;
providing an initial population of T-lymphocytes,
increasing the intracellular concentration of a memory induction compound in the T-lymphocytes, and
culturing the T-lymphocytes,
thereby producing an expanded population of T-lymphocytes,
wherein the memory induction compound has the formula (I); 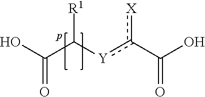
wherein:
p is 0 or 1, and when p is 0, Y is —CH₂— or —C═, and when p is 1, Y is selected from —CH—, CH₂, —NH—, —S, and —O—;
—R¹ is —H, —(CH₂)ₙCH₃, —(CH₂)ₙCH₂CO₂H, —CH₂Ph or —CH₂PhOCH₂Ph;
and when Y is —CH—, CH₂, —NH—, —S, or —O—, X is a single bonded group selected from —H, —OH, —NH₂, —SH, —(CH₂)ₙCH₃—(CH₂)ₙCH₂CO₂H, —F, —Cl, —Br, and —I, or a double bonded group selected from ═O and ═S;
and when Y is a double bonded —C═, X is —H; and
each n is independently 0 to 12,
and the mono- and diester forms thereof, such as the alkyl mono- and diester forms thereof.

2.-4. (canceled)

5. A method according to claim 1 wherein the memory-like T-lymphocytes have a phenotype which comprises CD62Lhigh, CCR7high and CD44high.

6. (canceled)

7. A method according to claim 1 wherein the T-lymphocytes in the initial population are polyclonal.

8. A method according to claim 7 wherein the initial population of T-lymphocytes are tumor infiltrating lymphocytes (TILs).

9. (canceled)

10. A method according to claim 1 wherein the T-lymphocytes in the initial population are monoclonal.

11. A method according to claim 10 wherein the method comprises modifying the T-lymphocytes to express a heterologous antigen receptor.

12.-17. (canceled)

18. A method according to claim 1 wherein the memory induction compound has the formula (II): 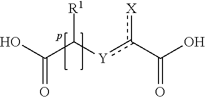
wherein:
p is 1;
Y is selected from —CH—, CH₂, —NH—, —S, and —O—;
—R¹ is —H;
X is a single bonded group selected from —H, —OH, —NH₂, —SH, —(CH₂)ₙCH₃—(CH₂)ₙCH₂CO₂H, —F, —Cl, —Br, and —I; and
each n is independently 0 to 12,
and the mono- and diester forms thereof, such as the alkyl mono- and diester forms thereof.

19. A method according to claim 1 wherein the memory induction compound has the formula (III): 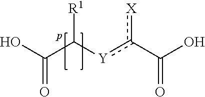
wherein:
p is 1;
—R¹ is —H, —(CH₂)ₙCH₃, —(CH₂)ₙCH₂CO₂H, —CH₂Ph or —CH₂PhOCH₂Ph;
Y is selected from —CH—, CH₂, —NH—, —S, and —O—;
X is a double bonded group selected from ═O and ═S; and
each n is independently 0 to 12,
and the mono- and diester forms thereof, such as the alkyl mono- and diester forms thereof.

20. A method according to claim 1 wherein the memory induction compound has the formula (IV); 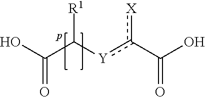
wherein:
p is 0;
X is H; and
Y is selected from —CH—, CH₂, —NH—, —S, and —O—,
and the mono- and diester forms thereof, such as the alkyl mono- and diester forms thereof.

21. A method according to claim 1 wherein the memory induction compound is selected from the group consisting of S-2-hydroxyglutarate (S-2HG), R-2-hydroxyglutarate (R-2HG), succinate and fumarate.

22.-24. (canceled)

25. A method according to claim 1 wherein the intracellular concentration of the memory induction compound in the T-lymphocytes is increased by culturing the T-lymphocytes in a medium that comprises the memory induction compound or a pro-form of the memory induction compound.

26.-28. (canceled)

29. A method according to claim 25 wherein the pro-form comprises the memory induction compound conjugated to a cell permeable moiety.

30. A method according to claim 29 wherein the cell permeable moiety is a C1 to C12 alkyl group.

31.-33. (canceled)

34. A method according to claim 30 wherein the pro-form is S-2HG octyl ester, R-2HG octyl ester, dimethylsuccinate or monomethylfumarate.

35. A method according to claim 1 wherein the method comprises isolating the memory-like T-lymphocytes following said culturing.

36. A method according to claim 35 wherein the method comprises formulating the memory-like T-lymphocytes into a pharmaceutical composition with a pharmaceutically acceptable excipient.

37. (canceled)

38. (canceled)

39. A method according to claim 35 wherein the expanded population of T-lymphocytes is administered to a recipient individual.

40.-43. (canceled)

44. A method according to claim 39 wherein the recipient individual has a cancer condition, infection or autoimmune disease.

45. (canceled)

46. (canceled)

47. A method of treatment of a disease that is ameliorated by a T-lymphocyte mediated immune response comprising;
providing an initial population of T-lymphocytes obtained from a donor individual,
increasing the intracellular concentration of a memory induction compound in the T-lymphocytes and
culturing the T-lymphocytes having increased intracellular concentration of the memory induction compound to produce an expanded population of memory-like T-lymphocytes, and;
administering the expanded population of memory-like T-lymphocytes to a recipient individual.

48.-50. (canceled)

51. A method according to claim 47 wherein the disease is a viral, fungal or bacterial infection, a cancer condition or an autoimmune disease.

52.-61. (canceled)


SEQUENCE LISTING (5 sequences)

SEQ ID NO: 1 - protein, 8 aa, Artificial Sequence
  Peptide comprising ovalbumin residues
SIINFEKL

SEQ ID NO: 2 - protein, 11 aa, Artificial Sequence
  trans-activating transcriptional activator peptide
YGRKKRRQRRR

SEQ ID NO: 3 - protein, 16 aa, Artificial Sequence
  Penetratin peptide
RQIKIYFQNRRMKWKK

SEQ ID NO: 4 - protein, 9 aa, Artificial Sequence
  CAR peptide
CARSKNKDC

SEQ ID NO: 5 - protein, 7 aa, Artificial Sequence
  oligoarginine Xentry peptide
LCLRPVG